(19) United States
(12) Patent Application Publication
(10) Pub. No.: US 2007/0185106 A1
(43) Pub. Date: Aug. 9, 2007

(54) Pyrrole derivatives as gonadotropin releasing hormone (gnrh) antagonists

(75) Inventor: Craig Steven Harris, Reims (FR)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 10/598,117

(22) PCT Filed: Feb. 17, 2005
(86) PCT No.: PCT/GB05/00560

§ 371 Date: Sep. 12, 2006

(30) Foreign Application Priority Data
Feb. 20, 2004 (EP) 04290467.2

(51) Int. Cl. A61K 31/5355; A61K 31/44; C07D 403/04; A61K 31/445

(52) U.S. Cl. 514/235.5; 514/326; 514/340; 514/413; 544/141; 546/247; 546/268.1; 548/465

(57) ABSTRACT

The invention relates to a group of novel thieno-pyrrole compounds of formula (I) wherein: R¹, R², R³, R⁴ M, and R⁵ are as defined in the specification, as inter alia, gonadotrophin releasing hormone antagonists. Novel compounds of formula (I) are also claimed. The invention also relates to pharmaceutical formulations of said compounds, methods of treatment using said compounds and to processes for the preparation of said compounds.

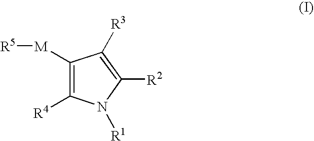

The present invention relates to compounds which are antagonists of gonadotropin releasing hormone (GnRH) activity. The invention also relates to pharmaceutical formulations, the use of a compound of the present invention in the manufacture of a medicament, a method of therapeutic treatment using such a compound and processes for producing the compounds.

Gonadotropin releasing hormone (GnRH) is a decapeptide that is secreted by the hypothalamus into the hypophyseal portal circulation in response to neural and/or chemical stimuli, causing the biosynthesis and release of luteinizing hormone (LH) and follicle-stimulating hormone (FSH) by the pituitary. GnRH is also known by other names, including gonadoliberin, LH releasing hormone (LHRH), FSH releasing hormone (FSH RF) and LH/FSH releasing factor (LH/FSH RF).

GnRE plays an important role in regulating the action of LH and FSH (by regulation of their levels), and thus has a role in regulating the levels of gonadal steroids in both sexes, including the sex hormones progesterone, oestrogens and androgens. More discussion of GnRH can be found in WO 98/55119 and WO 97/14697, the disclosures of which are incorporated herein by reference.

It is believed that several diseases would benefit from the regulation of GnRH activity, in particular by antagonising such activity. These include sex hormone related conditions such as sex hormone dependent cancer, benign prostatic hypertrophy and myoma of the uterus. Examples of sex hormone dependent cancers are prostatic cancer, uterine cancer, breast cancer and pituitary gonadotrophe adenoma.

The following disclose compounds purported to act as GnRH antagonists: WO 97/21435, WO 97/21703, WO 97/21704, WO 97/21707, WO 55116, WO 98/55119, WO 98/55123, WO 98/55470, WO 98/55479, WO 99/21553, WO 99/21557, WO 99/41251, WO 99/41252, WO 00/04013, WO 00/69433, WO 99/51231, WO 99/51232, WO 99/51233, WO 99/51234, WO 99/51595, WO 99/51596, WO 00/53178, WO 00/53180, WO 00/53179, WO 00/53181, WO 00/53185, WO 00/53602, WO 02/066477, WO 02/066478, WO 02/06645 and WO 02/092565. WO 2004/017961, which was published after the priority date of the present application contains further examples of such compounds.

It would be desirable to provide further compounds, such compounds being GnRH antagonists. Thus, according to the first aspect of the invention there is provided the use of a compound of Formula (I), 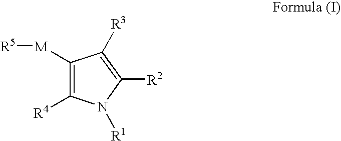
wherein:
R¹ is selected from: hydrogen, optionally substituted C₁₋₆alkyl, optionally substituted aryl or optionally substituted arylC₁₋₆alkyl, wherein the optional substituents are selected from C₁₋₄alkyl, nitro, cyano, fluoro and C₁₋₄alkoxy;
R² is an optionally substituted mono or bi-cyclic aromatic ring, wherein the optional substituents are 1, 2 or 3 substituents independently selected from: cyano, R^{e}R^{f}N—, C₁₋₆alkyl, C₁₋₆alkoxy, halo, haloC₁₋₆alkyl or haloC₁₋₆alkoxy wherein R^{e} and R^{f} are independently selected from hydrogen, C₁₋₆alkyl or aryl;
R³ is selected from a group of Formula (IIa) to Formula (IId): 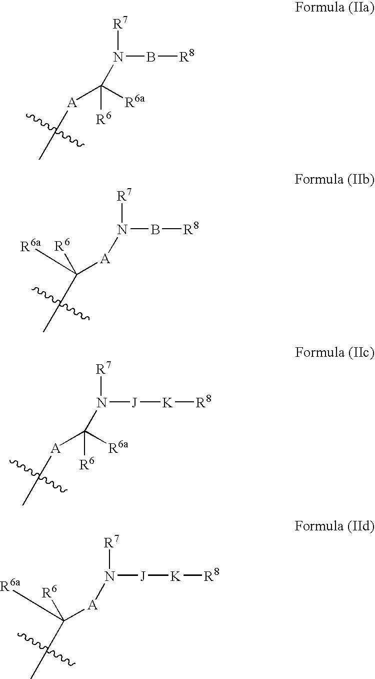
where R⁶ and R^{6a} are independently selected from hydrogen, fluoro, optionally substituted C₁₋₆alkyl, C₁₋₆alkoxy, or R⁶ and R^{6a} taken together and the carbon atom to which they are attached form a carbocyclic ring of 3-7 atoms or R⁶ and R^{6a} taken together and the carbon atom to which they are attached form a carbonyl group;
or when A is not a direct bond the group 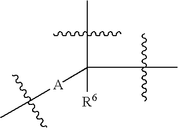
   forms a carbocyclic ring of 3-7 carbon atoms or a heterocyclic ring containing one or more heteroatoms;
or the group 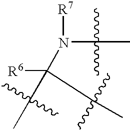
   forms a heterocyclic ring containing 3-7 carbon atoms and one or more heteroatoms;
R⁷ is selected from: hydrogen or C₁₋₆alkyl;
R⁸ is selected from:
   (i) hydrogen, C₁₋₆alkyl, C₂₋₆alkenyl, C₂₋₆alkynyl, haloC₁₋₆alkyl, C₁₋₄alkoxyC₁₋₄alkyl, hydroxy, hydroxyC₁₋₆alkyl, cyano, N—C₁₋₄alkylamino, N,N-di-C₁₋₄alkylamino, C₁₋₆alkyl-S(Oₙ)—, —O—R^{b}, —NR^{b}R^{c}, —C(O)—R^{b}, —C(O)O—R^{b}, —CONR^{b}R^{c}, NH—C(O)—R^{b} or —S(Oₙ)NR^{b}R^{c},
      where R^{b} and R^{c} are independently selected from hydrogen and C₁₋₆alkyl (e.g. C₁₋₄alkyl) optionally substituted with hydroxy, amino, N—C₁₋₄alkylamino, N,N-di-C₁₋₄alkylamino, HO—C₂₋₄alkyl-NH— or HO—C₂₋₄alkyl-N(C₁₋₄alkyl)-;
   (ii) nitro when B is a group of Formula (IV) and X is CH and p is 0;
   (iii) carbocyclyl (such as C₃₋₇cycloalkyl or aryl) or arylC₁₋₆alkyl each of which is optionally substituted by R¹², or R¹³;
   (iv) heterocyclyl or heterocyclylC₁₋₆alkyl each of which is optionally substituted by up to 4 substituents independently selected from R¹² or R¹³, and where any nitrogen atoms within a heterocyclyl group are, where chemically allowed, optionally in their oxidised (N→O, N—OH) state;
A is selected from:
   (i) a direct bond;
   (ii) optionally substituted C₁₋₅alkylene wherein the optional substituents are independently selected from: hydroxy, hydroxyC₁₋₆alkyl, C₁₋₆alkyl, C₁₋₆alkyloxy, C₁₋₄alkoxyC₁₋₄alkyl, aryl or arylC₁₋₆alkyl;
   (iii) a carbocyclic ring of 3-7 atoms;
   (iv) a carbonyl group or —C(O)—C(R^{d}R^{d})—, wherein R^{d} is independently selected from hydrogen and C₁₋₂alkyl;
or when R³ is a group of Formula (IIa) or (IIb), the group 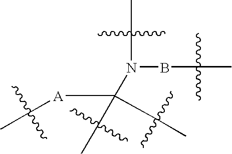
   forms a heterocyclic ring containing 3-7 carbon atoms and one or more heteroatoms;
or when R³ is a group of Formula (IIa), (IIb), (IIc) or (IId), the group 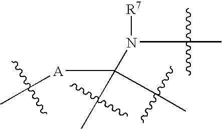
   forms a heterocyclic ring containing 3-7 carbon atoms and one or more heteroatoms;
B is selected from:
   (i) a direct bond;
   (ii) a group of Formula (IV) 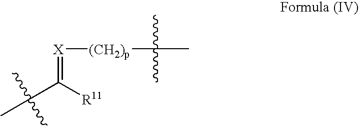
   wherein:
   X is selected from N or CH,
      wherein at position (a) Formula (IV) is attached to the nitrogen atom and the (CH₂)ₚ group is attached to R⁸; and
   (iii) a group independently selected from: optionally substituted C₁₋₆alkylene, optionally substituted C₃₋₇cycloalkyl, optionally substituted C₃₋₆alkenylene, optionally substituted C₃₋₆alkynyl, (C₁₋₅alkyl)ₐₐ-S(Oₙ)—(C₁₋₅alkyl)_{bb}-, —(C₁₋₅alkyl)ₐₐ-O—(C₁₋₅alkyl)_{bb}-, —(C₁₋₅alkyl)ₐₐ-C(O)—(C₁₋₅alkyl)_{bb}- or (C₁₋₅alkyl)ₐₐ-N(R¹⁷)—(C₁₋₅alkyl)_{bb}, or —(C₁₋₅alkyl)ₐₐ-C(O)NH—(C₁₋₅alkyl)_{bb}-
   where R¹⁷ is hydrogen or C₁₋₄alkyl, or where R¹⁷ and the (C₁₋₅alkyl)ₐₐ or (C₁₋₅alkyl)_{bb} chain can be joined to form a heterocyclic ring, wherein aa and bb are 0 or 1 and the combined length of (C₁₋₅alkyl)ₐₐ and (C₁₋₅alkyl)_{bb} is less than or equal to C₅alkyl and wherein the optional substituents are independently selected from R¹²;
   or the group —B—R⁸ represents a group of Formula (V) 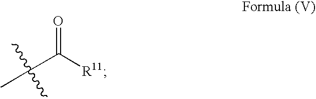
      or the group 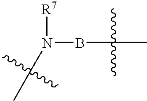
      together forms an optionally substituted heterocyclic ring containing 4-7 carbons atoms, wherein the optional substituents are selected from 1 or 2 substituents independently selected from R¹² and R¹³;
or the group 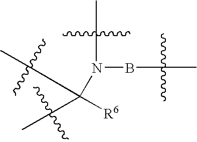
   forms a heterocyclic ring containing 3-7 carbon atoms and one or more heteroatoms;

R¹¹ is selected from: hydrogen, optionally substituted C₁₋₆alkyl, N(R²³R²⁴) or NC(O)OR²⁵, where R²³, R²⁴ and R²⁵ are independently selected from: hydrogen, hydroxy, optionally substituted C₁₋₆alkyl, optionally substituted aryl, optionally substituted arylC₁₋₆alkyl, an optionally substituted carbocyclic ring of 3-7 atoms, optionally substituted heterocyclyl or optionally substituted heterocyclylC₁₋₆alkyl or R²³ and R²⁴ taken together with the nitrogen atom to which they are attached, can form an optionally substituted ring of 3-10 atoms, wherein the optional substituents are selected from R¹² and 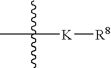
where K and R⁸ are as defined herein;

J is a group of the formula: —(CH₂)ₛ-L-(CH₂)ₛ— or —(CH₂), —C(O)—(CH₂)ₛ-L-(CH₂)ₛ—, wherein when s is greater than 0, the alkylene group is optionally substituted,

or the group 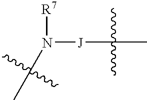
together forms an optionally substituted heterocyclic ring containing 4-7 carbons atoms, wherein the optional substituents are selected from 1 or 2 substituents independently selected from R¹² and R¹³;

K is selected from: a direct bond, —(CH₂)ₛ₁—, —(CH₂)ₛ₁—O—(CH₂)ₛ₂—, —(CH₂)ₛ₁—C(O)—(CH₂)ₛ₂—, —(CH₂)ₛ₁—S(Oₐ)—(CH₂)ₛ₂—, —(CH₂)ₛ₁—N(R^{17a})—(CH₂)ₛ₂—, —(CH₂)ₛ₁—C(O)N(R^{17a})—(CH₂)ₛ₂—, —(CH₂)ₛ₁—N(R^{17a})C(O)—(CH₂)ₛ₂—, —(CH₂)ₛ₁—N(R^{17a})C(O)N(R^{17a})—(CH₂)ₛ₂, —(CH₂)ₛ₁—OC(O)—(CH₂)ₛ₂—, —(CH₂)ₛ₁—C(O)O—(CH₂)ₛ₂—, —(CH₂)ₛ₁—N(R^{17a})C(O)O—(CH₂)ₛ₂—, —(CH₂)ₛ₁—OC(O)N(R^{17a})—(CH₂)ₛ₂—, —(CH₂)ₛ₁—OS(Oₙ)—(CH₂)ₛ₂—, or —(CH₂)ₛ₁—S(Oₙ)—O—(CH₂)ₛ₂—, —(CH₂)ₛ₁—S(O)₂N(R^{17a})—(CH₂)ₛ₂— or —(CH₂)ₛ₁—N(R^{17a})S(O)₂—(CH₂)ₛ₂—; wherein the —(CH₂)ₛ₁— and —(CH₂)ₛ₂— groups are independently optionally substituted by hydroxy or C₁₋₄alkyl and wherein when s1>1 or s2>1 then the CH₂ group can optionally be a branched chain;
where R^{17a} is hydrogen or C₁₋₄alkyl;
L is selected from optionally substituted aryl or optionally substituted heterocyclyl;
R⁴ is selected from hydrogen, C₁₋₄alkyl or halo;
R⁵ is selected from a group of Formula III-a; III-b; III-c; III-d; III-e; III-f, III-g, III-h, III-i, or III-j, III-k, III-l, III-m, III-n or III-o 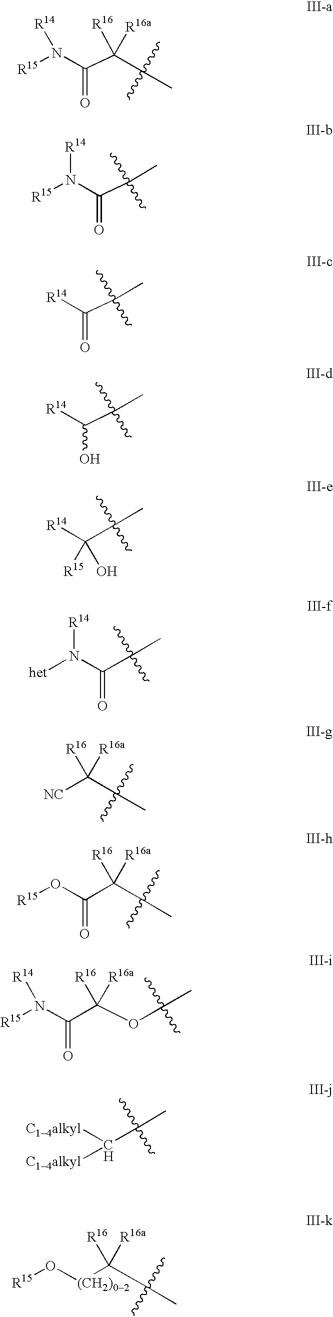 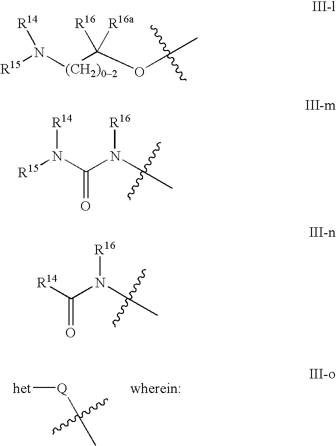
   het represents an optionally substituted 3- to 8-membered heterocyclic ring containing from 1 to 4 heteroatoms independently selected from O, N and S, wherein the optional substituents are selected from 1-2 groups selected from R¹² and R¹³; and
   Q is selected from a direct bond or —[C(R¹⁶R^{16a})]₁₋₂—;
R¹⁴ and R¹⁵ are selected from:
   (i) R¹⁴ selected from hydrogen; optionally substituted C₁₋₈alkyl; optionally substituted aryl; —R^{d}—Ar, where R^{d} represents C₁₋₈alkylene and Ar represents optionally substituted aryl; and optionally substituted 3- to 8-membered heterocyclic ring optionally containing from 1 to 3 further heteroatoms independently selected from O, N and S; and R¹⁵ is selected from hydrogen; optionally substituted C₁₋₈alkyl and optionally substituted aryl;
   (ii) wherein the group of Formula (III) represents a group of Formula III-a, III-b, III-i, III-l or III-m, then the group NR¹⁴(—R¹⁵) represents an optionally substituted 3- to 8-membered heterocyclic ring optionally containing from 1 to 3 further heteroatoms independently selected from O, N and S; or
   (iii) wherein the group of Formula (III) represents structure III-e, 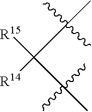
      represents an optionally substituted 3- to 8-membered heterocyclic ring optionally containing from 1 to 4 heteroatoms independently selected from O, N and S;
R¹⁶ and R^{16a} are independently selected from:
   (i) hydrogen or optionally substituted C₁₋₈alkyl; or
   (ii) R¹⁶ and R^{16a} together with the carbon to which they are attached form an optionally substituted 3 to 7-membered cycloalkyl ring;
R¹² is independently selected from: halo, hydroxy, hydroxyC₁₋₆alkyl, oxo, cyano, cyanoC₁₋₆alkyl, nitro, carboxyl, C₁₋₆alkyl, C₁₋₆alkoxy, C₁₋₆alkoxyC₁₋₄alkyl, C₁₋₆alkoxycarbonylC₀₋₄alkyl, C₁₋₆alkanoylC₀₋₄alkyl, C₁₋₆alkanoyloxyC₀₋₄alkyl, C₂₋₆alkenyl, C₁₋₃perfluoroalkyl-, C₁₋₃perfluoroalkoxy, aryl, arylC₁₋₆alkyl, heterocyclyl, heterocyclylC₁₋₆alkyl, aminoC₀₋₄alkyl, N—C₁₋₄alkylaminoC₀₋₄alkyl, N,N-di-C₁₋₄alkylaminoC₀₋₄alkyl, carbamoyl, N—C₁₋₄alkylcarbamoylC₀₋₂alkyl, N,N-di-C₁₋₄alkylaminocarbamoylC₀₋₂alkyl, aminocarbonylC₀₋₄alkyl, N—C₁₋₆alkyaminocarbonylC₀₋₄alkyl, N,N—C₁₋₆alkyaminocarbonylC₀₋₄alkyl, C₁₋₆alkyl-S(O)ₙ-aminoC₀₋₄alkyl-, aryl-S(O)ₙ-aminoC₀₋₂alkyl-, C₁₋₃perfluoroalkyl-S(O)ₙ-aminoC₀₋₂alkyl-; C₁₋₆alkylamino-S(O)ₙ—C₀₋₂alkyl-, arylamino-S(O)ₙ—C₀₋₂alkyl-, C₁₋₃perfluoroalkylamino-S(O)ₙ—C₀₋₂alkyl-, C₁₋₆alkanoylamino-S(O)ₙ—C₀₋₂alkyl-; arylcarbonylamino-S(O)ₙ—C₀₋₂alkyl-, C₁₋₆alkyl-S(O)ₙ—C₀₋₂alkyl-, aryl-S(O)ₙ—C₀₋₂alkyl-, C₁₋₃perfluoroalkyl-, C₁₋₃perfluoroalkoxyC₀₋₂alkyl; R⁹′OC(O)(CH₂)_{w}—, R⁹″R¹⁰″N(CH₂)_{w}—, R⁹′R¹⁰′NC(O)(CH₂)_{w}—, R⁹R¹⁰NC(O)N(R⁹)(CH₂)_{w}—, R⁹OC(O)N(R⁹)(CH₂)_{w}—, or halo, wherein w is an integer between 0 and 4 and R⁹ and R¹⁰ are independently selected from hydrogen, C₁₋₄alkyl, C₁₋₄alkylsulphonyl and C₃₋₇-carbocyclyl, R⁹′ and R¹⁰′ are independently selected from C₁₋₄alkylsulplhonyl and C₃₋₇carbocyclyl, and R⁹″ and R¹⁰″ are C₃₋₇carbocyclyl; wherein an amino group within R¹² is optionally substituted by C₁₋₄alkyl;
   R¹³ is C₁₋₄alkylaminocarbonyl wherein the alkyl group is optionally substituted by 1, 2 or 3 groups selected from R¹², or R¹³ is a group —C(O)—R¹⁸ and R¹⁸ is selected from an amino acid derivative or an amide of an amino acid derivative;
M is selected from —CH₂—CH₂— or —CH═CH—;
n is an integer from 0 to 2;
p is an integer from 0 to 4;
s, s1 and s2 are independently selected from an integer from 0 to 4, and
s1+s2 is less than or equal to 4;
t is an integer between 0 and 4; and
   or a salt, solvate or pro-drug thereof, in the manufacture of a medicament for
(a) antagonising gonadotropin releasing hormone activity;
(b) administration to a patient, for reducing the secretion of luteinizing hormone by the pituitary gland of the patient; and
(c) administration to a patient, for therapeutically treating and/or preventing a sex hormone related condition in the patient, preferably a sex hormone related condition selected from prostate cancer and pre-menopausal breast cancer.

Compounds of formula (I) are novel and therefore these form a further aspect of the invention.

In a particular embodiment, the invention provides a compound of formula (IA) which is a compound of formula (I) as defined above,

with the proviso that when

(i) the group 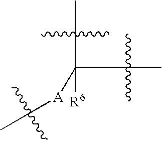
   forms an aromatic carbocyclic ring of 3-7 carbon atoms or an aromatic heterocyclic ring containing one or more heteroatoms, or
(ii) when R³ is a group of Formula (IIa) or (IIb), and the group 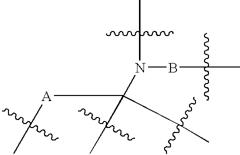
   forms an aromatic heterocyclic ring containing 3-7 carbon atoms and one or more heteroatoms; or
(iii) when R³ is a group of Formula (IIa), (IIb), (IIc) or (IId), and the group 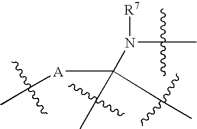
   forms an aromatic heterocyclic ring containing 3-7 carbon atoms and one or more heteroatoms, or
(iv) when the group 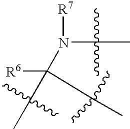
   forms an aromatic heterocyclic ring containing 3-7 carbon atoms and one or more heteroatoms and A is a direct bond;
   then R⁵ is other than a group III-o.

Preferably, the group A is selected from (i) a direct bond or (ii) optionally substituted C₁₋₅alkylene wherein the optional substituents are independently selected from: hydroxy, hydroxyC₁₋₆alkyl, C₁₋₆alkyl, C₁₋₆alkoxy, C₁₋₄alkoxyC₁₋₄alkyl, aryl or arylC₁₋₆alkyl.

Most preferably, the group A is selected from a group (ii) above.

In a further embodiment of the invention there is a provided a compound of Formula (I) or (IA) as defined above which includes a group R¹³ and wherein the group R¹³ is —C(O)—R¹⁸, and

R¹⁸ is selected from an amino acid derivative or an amide of an amino acid derivative;

or a salt, solvate or pro-drug thereof.

According to a further feature of the first aspect of the invention there is provided a pharmaceutical formulation comprising a compound of Formula (IA), or salt, pro-drug or solvate thereof, and a pharmaceutically acceptable diluent or carrier.

According to a further feature of the first aspect of the invention there is provided the following uses of a compound of Formula (I) or (IA), or salt, pro-drug or solvate thereof:
(a) the use in the manufacture of a medicament for antagonising gonadotropin releasing hormone activity;
(b) the use in the manufacture of a medicament for administration to a patient, for reducing the secretion of luteinizing hormone by the pituitary gland of the patient; and
(c) the use in the manufacture of a medicament for administration to a patient, for therapeutically treating and/or preventing a sex hormone related condition in the patient, preferably a sex hormone related condition selected from prostate cancer and pre-menopausal breast cancer.

According to a further aspect of the invention there is provided a method of antagonising gonadotropin releasing hormone activity in a patient, comprising administering a compound of Formula (I) or (IA), or salt, pro-drug or solvate thereof, to a patient.

Whilst pharmaceutically-acceptable salts of compounds of the invention are preferred, other non-pharmaceutically-acceptable salts of compounds of the invention may also be useful, for example in the preparation of pharmaceutically-acceptable salts of compounds of the invention.

Whilst the invention comprises compounds of the invention, and salts, pro-drugs or solvates thereof, in a further embodiment of the invention, the invention comprises compounds of the invention and salts thereof.

In the present specification, unless otherwise indicated, an alkyl, alkylene, alkenyl or alkynyl moiety may be linear or branched. The term “alkylene” refers to the group —CH₂—. Thus, C₈ alkylene for example is —(CH₂)₈—. For avoidance of doubt the term C₀alkyl within the group C₀₋₅alkyl is a direct bond.

The term ‘propylene’ refers to trimethylene and the branched alkyl chains —CH(CH₃)CH₂— and —CH₂—CH(CH₃)—. The straight chain propylene di-radical is preferred, i.e. —CH₂CH₂CH₂—. Specific propylene radicals refer to the particular structure, thus the term, propyl-2-ene refers to the group —CH₂—CH(CH₃)—. Similar notation is used for other divalent allyl chains such as butylene.

The term ‘2-propenyl’ refers to the group —CH₂—CH═CH—.

The term “aryl” refers to phenyl or naphthyl.

The term “carbamoyl” refers to the group —C(O)NH₂.

The term “halo” refers to fluoro, chloro, bromo or iodo.

The term “heterocyclyl” or “heterocyclic ring” refers to a 4-12 membered, preferably 5-10 membered aromatic mono or bicyclic ring or a 4-12 membered, preferably 5-10 membered saturated or partially saturated mono or bicyclic ring, said aromatic, saturated or partially unsaturated rings containing up to 5 heteroatoms independently selected from nitrogen, oxygen or sulphur, linked via ring carbon atoms or ring nitrogen atoms where a bond from a nitrogen is allowed, for example no bond is possible to the nitrogen of a pyridine ring, but a bond is possible through the 1-nitrogen of a pyrazole ring. Examples of 5- or 6-membered aromatic heterocyclic rings include pyrrolyl, furanyl, imidazolyl, triazolyl, pyrazinyl, pyrimidinyl, pyridazinyl, pyridinyl, isoxazolyl, oxazolyl, 1,2,4 oxadiazolyl, isothiazolyl, thiazolyl and thienyl. A 9 or 10 membered bicyclic aromatic heterocyclic ring is an aromatic bicyclic ring system comprising a 6-membered ring fused to either a 5 membered ring or another 6 membered ring. Examples of 5/6 and 6/6 bicyclic ring systems include benzofuranyl, benzimidazolyl, benzthiophenyl, benzthiazolyl, benzisothiazolyl, benzoxazolyl, benzisoxazolyl, indolyl, pyridoimidazolyl, pyrimidoimidazolyl, quinolinyl, isoquinolinyl, quinoxalinyl, quinazolinyl, phthalazinyl, cinnolinyl and naphthyridinyl. Examples of saturated or partially saturated heterocyclic rings include pyrrolinyl, pyrrolidinyl, morpholinyl, piperidinyl, piperazinyl, dihydropyridinyl, benzodioxyl and dihydropyrimidinyl. This definition further comprises sulphur-containing rings wherein the sulphur atom has been oxidised to an S(O) or S(O2) group.

The term “aromatic ring” refers to a 5-10 membered aromatic mono or bicyclic ring optionally containing up to 5 heteroatoms independently selected from nitrogen, oxygen or sulphur. Examples of such “aromatic rings” include: phenyl, naphtyl, pyrrolyl, pyrazolyl, furanyl, imidazolyl, triazolyl, pyrazinyl, pyrimidinyl, pyridazinyl, pyridinyl, isoxazolyl, oxazolyl, 1,2,4 oxadiazolyl, isothiazolyl, thiazolyl and thienyl. Preferred aromatic rings include phenyl, thienyl and pyridyl.

The term “carbocyclyl” or “carbocyclic ring” includes rings of carbon atoms, for example of from 3-12 carbon atoms, which may be saturated, unsaturated (such as aryl or aromatic rings such as phenyl or naphthyl, as described above) or partially unsaturated. They may be mono- or bi-cyclic.

The term “amino acid derivative” is defined as that derived from the coupling of an L- or D-amino acid with a carboxyl group via an amide bond. This bond is formed via the amino group on the amino acid backbone. Amino acid residues include those derived from natural and non-natural amino acids, preferably natural amino acids and include α-amino acids β-amino acids and γ-amino acids. For the avoidance of doubt amino acids include those with the generic structure: 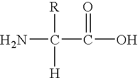
where R is the amino acid side chain. The definition of amino acid also includes amino acid analogues which have additional methylene groups within the amino acid backbone, for example β-alanine and amino acids which are not naturally occurring such as cyclohexylalanine.

Preferred amino acids include glycine, alanine, valine, leucine, isoleucine, methionine, proline, phenylalanine, tryptophan, serine, threonine, cysteine, tyrosine, asparaginine, glutamine, aspartic acid, glutamic acid, lysine, histidine, β-alanine and ornithine. More preferred amino acids include glutamic acid, serine, threonine, glycine, alanine, β-alanine and lysine. Yet more preferred amino acids include: alanine, asparagine, glycine, leucine, methionine, serine and threonine and non-natural amino acids with the following side chains: CH₃—S—CH₂—, CH₃—CH₂—, CH₃—CH(OH)— and HO—CH₂CH₂—.

Especially preferred amino acids include alanine, leucine, methionine and serine and non-natural amino acids with the following side chains: CH₃—S—CH₂—, CH₃—CH₂—, CH₃—CH(OH)— and HO—CH₂CH₂—.

An amide of an amino acid is defined as amino acid as defined above wherein the carboxy group on the amino acid backbone has been converted to an amide, or where present the carboxyl group on an amino acid side chain has been converted to an amide. Optionally the amino group of the amide group is substituted by C₁₋₄alkyl.
For example, the equivalent generic structure to the generic amino structure above is:

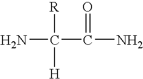

The Symbol 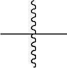
denotes where the respective group is linked to the remainder of the molecule.

For the avoidance of doubt where two groups or integers appear within the same definition, for example, —(CH₂)ₛ-L-(CH₂)ₛ—, then these can be the same or different.

For the avoidance of doubt, where several groups together form a ring, for example: ‘the group 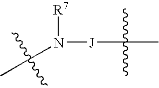
together forms an optionally substituted heterocyclic ring containing 4-7 carbon atoms’, then the groups shown cyclises to form a ring, i.e 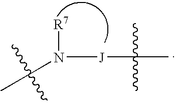
For example in Example 5 this group forms a piperazine ring.

The term C₁₋₃perfluoroalkyl refers to a C₁₋₃alkyl chain in which all hydrogens have been replaced with a fluorine atom. Examples of C₁₋₃perfluoroalkyl include trifluoromethyl, pentafluoroethyl and 1-trifluoromethyl-1,2,2,2-tetrafluoroethyl. Preferably C₁₋₃perfluoroalkyl is trifluoromethyl.

Examples of C₁₋₈alkyl include: methyl, ethyl, propyl, isopropyl, butyl, iso-butyl, tert-butyl and 2-methyl-pentyl; examples of C₁₋₈alkylene include: methylene, ethylene and 2-methyl-propylene; examples of C₁₋₆alkenyl include allyl (2-propenyl) and 2-butenyl, examples of C₁₋₆alkynyl include 2-propynyl and 3-butynyl, examples of haloC₁₋₆alkyl include fluoroethyl, chloropropyl and bromobutyl, examples of hydroxyC₁₋₆alkyl include hydroxymethyl, hydroxyethyl and hydroxybutyl, examples of C₁₋₈alkoxy include methoxy, ethoxy and butyloxy; examples of C₁₋₄alkoxyC₁₋₄alkyl include methoxyethyl, propoxybutyl and propoxymethyl, examples of C₁₋₆alkanoyl include formyl, ethanoyl, propanoyl or pentanoyl, examples of N—C₁₋₄alkylamino include N-methylamino and N-ethylamino; examples of N,N-di-C₁₋₄alkylamino include N,N-dimethylaminoethyl, N,N-dimethylaminopropyl and N,N-dipropylaminoethyl, examples of HO—C₂₋₄alkyl-NH include hydroxymethylamino hydroxyethylamino and hydroxypropylamino, examples of HO—C₂₋₄alkyl-N(C₁₋₄alkyl) include N-methyl-hydroxymethylamino, N-ethyl-hydroxyethylamino, and N-propyl-hydroxypropylamino, examples of C₁₋₆alkyl-S(Oₙ)— include methylthio, methylsulphinyl, ethylsulphinyl, ethylsulphonyl and propylsulphonyl, examples of arylC₁₋₆alkyl include benzyl, phenethyl and phenylbutyl, examples of heterocyclylC₁₋₆alkyl include pyrrolidin-1-yl ethyl, imidazolylethyl, pyridylmethyl and pyrimidinylethyl.

It is to be understood that, insofar as certain of the compounds of the invention may exist in optically active or racemic forms by virtue of one or more asymmetric carbon atoms, the invention includes in its definition any such optically active or racemic form which possesses the property of antagonizing gonadotropin releasing hormone (GnRH) activity. The synthesis of optically active forms may be carried out by standard techniques of organic chemistry well known in the art, for example by synthesis from optically active starting materials or by resolution of a racemic form. Similarly, activity of these compounds may be evaluated using the standard laboratory techniques referred to hereinafter.

The invention also relates to any and all tautomeric forms of the compounds of the different features of the invention that possess the property of antagonizing gonadotropin releasing hormone (GnRH) activity.

It will also be understood that certain compounds of the present invention may exist in solvated, for example hydrated, as well as unsolvated forms. It is to be understood that the present invention encompasses all such solvated forms which possess the property of antagonizing gonadotropin releasing hormone (GnRH) activity.

Preferred compounds of Formula (I) or (IA) are those wherein any one of the following or any combination of the following apply.

Preferably R¹ is selected from hydrogen, optionally substituted C₁₋₆alkyl or optionally substituted arylC₁₋₆alkyl, wherein the optional substitutuents are as described herein. More preferably R¹ represents hydrogen, unsubstituted C₁₋₆alkyl or optionally substituted arylC₁₋₆alkyl. Yet more preferably R¹ represents hydrogen, methyl, ethyl, tert-butyl or benzyl. Most preferably R¹ represents hydrogen.

Preferably optional substituents on R¹ are independently selected from: fluoro and C₁₋₄alkoxy. Most preferably R¹ is unsubstituted.

Preferably R² is an optionally substituted monocyclic aromatic ring structure, wherein the optional substitutuents are as described herein. Most preferably R² represents optionally substituted phenyl, wherein the optional substitutuents are as described herein.

Preferably optional substituents on R² are independently selected from methyl, ethyl, ethoxy, ethoxy, tert-butoxy, F or Cl. Most preferably optional substituents on R² are independently selected from methyl, F or Cl. Preferably R² bears 1, 2 or 3 substituents, most preferably 2 substituents.

Most preferably R² represents

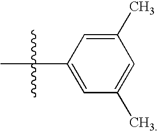

Preferably R³ is selected from a group of Formula (IIc) or Formula (IId). Most preferably R³ is a group of Formula (IId).

Preferably R⁴ is selected from hydrogen, methyl, ethyl, chloro or bromo. Further preferably R⁴ is selected from hydrogen or chloro. Most preferably R⁴ is hydrogen.

Preferably R⁵ is selected from a group of Formula III-a, III-g, III-h, III-i, III-j, III-k, III-l, or III-o 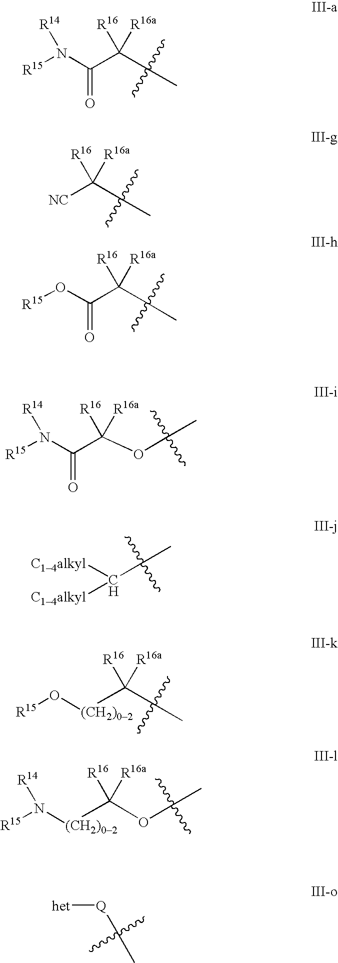
wherein R¹⁶, R^{16a}, R¹⁴ and R¹⁵ are as defined above.

More preferably the group of Formula (III) is selected from one of the following groups: 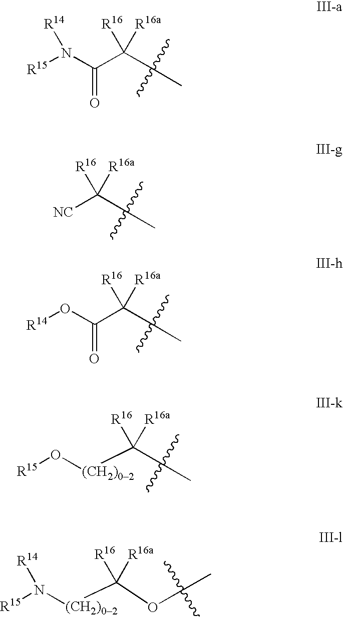
wherein R¹⁶, R^{16a}, R¹⁴ and R¹⁵ are as defined above.

Further preferably the group of Formula (III) is selected from one of the following groups: 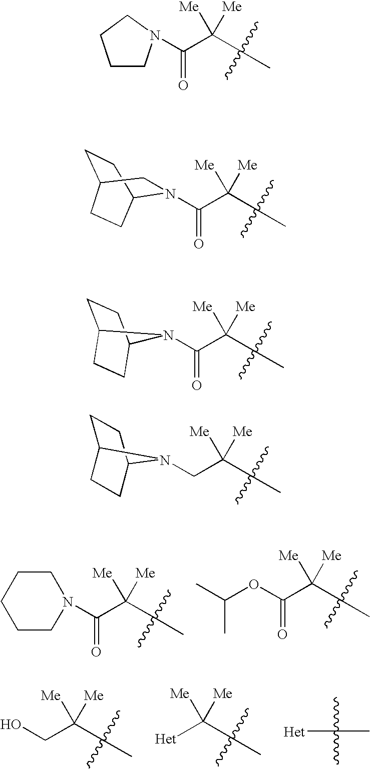
wherein Me represents methyl and het is as defined above.

Yet further preferably the group of Formula (III) is selected from one of the following groups:

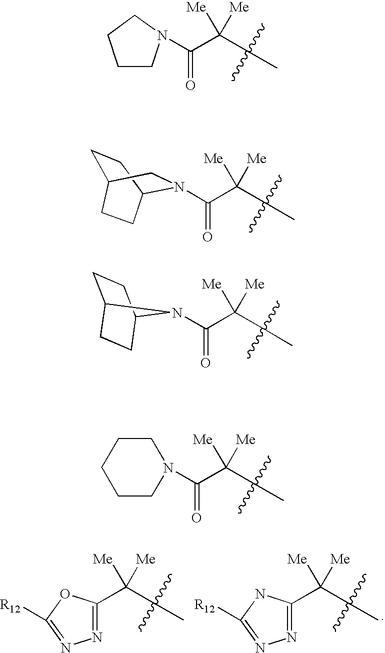

Most preferably the group of Formula (III) is:

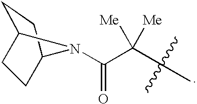

Suitably, R⁶ and R^{6a} are independently selected from hydrogen, fluoro, C₁₋₆alkyl, C₁₋₆alkoxy, or R⁶ and R^{6a} taken together and the carbon atom to which they are attached form a carbocyclic ring of 3-7 atoms or R⁶ and R^{6a} taken together and the carbon atom to which they are attached form a carbonyl group.

Preferably R⁶ and R^{6a} are independently selected from hydrogen, fluoro, optionally substituted C₁₋₆alkyl or R⁶ and R^{6a} taken together and the carbon atom to which they are attached form a carbocyclic ring of 3-7 atoms More preferably R⁶ and R^{6a} are independently selected from hydrogen, unsubstituted C₁₋₆alkyl or R⁶ and R^{6a} taken together and the carbon atom to which they are attached form a carbocyclic ring of 3-7 atoms. Yet more preferably R⁶ and R^{6a} are independently selected from hydrogen, methyl or R⁶ and R^{6a} taken together and the carbon atom to which they are attached form cyclopropyl. Most preferably R⁶ is hydrogen and R^{6a} is methyl.

Preferably R⁷ is selected from: hydrogen or C₁₋₄alkyl. More preferably R⁷ is hydrogen or methyl. Most preferably R⁷ is hydrogen.

Preferably R⁸ is selected from
(i) hydrogen, C₁₋₆alkyl, C₂₋₆alkenyl, haloC₁₋₆alkyl, hydroxy, cyano, C₁₋₆alkylS(Oₙ)—, —O—R^{b}, C₁₋₄alkoxyC₁₋₄alkyl, —C(O)—R^{b}, C(O)O—R^{b}, —NHC(O)—R^{b}, N,N-di-C₁₋₄alkylamino, —S(Oₙ)NR^{b}R^{c}
   where R^{b} and R^{c} are as defined above and are preferably independently selected from hydrogen and C₁₋₄alkyl, and n is 0, 1 or 2;
(ii) C₄₋₇heterocyclyl, optionally substituted by up to 3 groups selected from R¹² and R¹³, or
(iii) phenyl or C₃₋₇carbocyclyl; each of which is optionally substituted by up to 3 groups selected from R¹² and R¹³.

Particular examples of C₄₋₇heterocyclyl groups R⁸ include azirinyl, azetidinyl, pyrrolidinyl, pyrazolinyl, pyrazolidinyl, imidazolinyl, imidazolidinyl, piperidinyl, piperazinyl, hexahydropyrimidinyl, hexahydropyridazinyl, hexahydrotriazinyl, tetraydrotriazinyl, dihydrotriazinyl, tetrahydrofuranyl, dioxolanyl, tetrahydropyranyl, dioxanyl, trioxanyl, tetrahydrothienyl, 1-oxotetrahydrothienyl, 1,1-dioxotetrahydrothienyl tetrahydrothiopyran, 1-oxotetrahydrothiopyran, 1,1-dioxotetrahydrothiopyran, dithianyl, trithianyl, morpholinyl, oxathiolanyl, oxathianyl, thiomorpholinyl, thiazinanyl, 1-oxo-thiomorpholinyl, 1,1-dioxo-thiomorpholinyl, 1,1-dioxo-isothiazolidiyl, thiazolidinyl, pyrrolyl, imidazolyl, triazolyl, pyridyl, pyrimidinyl, pyrazinyl, pyridazinyl, triazinyl, thiazolyl, thiadiazolyl, thiadiazinyl, oxazolyl, isoxazolyl, oxadiazolyl, furazanyl, octahydropyrrolopyrrolyl, octahydropyrrolopyrrolyl, benzotriazolyl, dihydrobenzotriazolyl, indolyl, indolinyl, benzimidazolyl, 2,3-dihydrobenzimidazoly, benzotriazolyl 2,3-dihydro benzotriazolyl quinolinyl, isoquinolinyl, cinnolinyl, phthalazinyl, quinazolinyl, quinozalinyl, naphthyridinyl, pteridinyl, benzodioxolyl, tetrahydrodioxolopyrrolyl, 1,5-dioxa-9-azaspiro[5.5]undecanyl or 8-oxa-3-azabicyclooctanyl; each of which is optionally substituted by up to 3 groups selected from R¹² and R¹³.

Further preferably R⁸ is selected from
(i) hydrogen, methyl, isopropyl, t-butyl, 1-methylethyl, allyl, fluoroethyl, hydroxy, cyano, ethylsulphonyl, methoxy, 1-methyl-2-methoxyethyl, acetyl, t-butoxycarbonyl, acetylamino, dimethylamino, diethylamino, (1-methylethyl)amino, isopropylamino or aminosulphonyl;
(ii) azetidinyl, furanyl, tetrahydrofuranyl, tetrahydropyranyl, pyrrolidinyl, piperidinyl, piperazinyl, hexahydropyrimidinyl, morpholinyl, tetrahydrothienyl, 1,1-dioxotetrahydrothienyl, thiomorpholinyl, 1-oxo-thiomorpholinyl, 1,1-dioxo-thiomorpholinyl, imidazolyl, triazolyl, thienyl, thiazolyl, isoxazolyl, pyridyl, pyrimidinyl, pyrazinyl, tetrahydro-3aH-[1,3]dioxolo[4,5-c]pyrrolyl, 1,5-dioxa-9-azaspiro[5.5]undecanyl, 8-oxa-3-azabicyclo[3.2.1]octanyl, benzodioxolyl, 2,3-dihydrobenzotriazolyl, 1,1-dioxo-isothiazolidinyl 1,2-dihydroquinolinyl or octahydropyrrolo[3,4-c]pyrrolyl; each of which is optionally substituted by up to 3 groups selected from R¹² and R¹³; or
(iii) phenyl or C₃₋₇carbocyclyl, each of which is optionally substituted by up to 3 groups selected from R¹² and R¹³.

Yet further preferably R⁸ is selected from
(i) phenyl optionally substituted by up to 3 groups selected from R¹² and R¹³, or naphthyl;
(ii) furanyl, tetrahydropyranyl, pyrrolidinyl, piperazinyl, morpholinyl, 1,1-dioxo-thiomorpholinyl, thienyl, triazolyl, pyridyl, pyrimidinyl, pyrazinyl, tetrahydro-3aH-[1,3]dioxolo[4,5-c]pyrrolyl, benzodioxolyl, 1,2-dihydroquinolinyl, 1,1-dioxo-isothiazolidinyl or 2,3-dihydrobenzotriazolyl; each of which is optionally substituted by up to 3 groups selected from R¹² and R¹³; or
(iii) C₃₋₇carbocyclyl (preferably cyclohexyl or cyclopentyl, more preferably cyclohexyl) optionally substituted by up to 3 groups selected from R¹² and R¹³.

Further preferably R⁸ is selected from: phenyl, morpholino, piperidino, thienyl, pyridyl and benzodioxlyl optionally substituted by up to 3 groups selected from R¹² and R¹³.

Further preferably R⁸ is phenyl, morpholino, pyridyl, pyrrolidino, piperidino or 1,1-dioxo-isothiazolidin-2-yl or N-isopropylureido. Most preferably R⁸ is phenyl.

In a particular embodiment of the invention, optional substituents on R⁸ are selected from R¹² groups. Particular examples of R¹² are hydroxy, hydroxyC₁₋₆alkyl, oxo, cyano, cyanoC₁₋₆alkyl, nitro, carboxyl, C₁₋₆alkyl, C₁₋₆alkoxy, C₁₋₆alkoxyC₀₋₂alkyl, C₁₋₆alkoxycarbonylC₀₋₂alkyl, C₁₋₆alkanoylC₀₋₂alkyl, C₁₋₆alkanoyloxyC₀₋₂alkyl, C₂₋₆alkenyl, C₁₋₃perfluoroalkyl-, C₁₋₃perfluoroalkoxy, aryl, arylC₁₋₆alkyl, heterocyclyl, heterocyclylC₁₋₆alkyl, N—C₁₋₄alkylaminoC₀₋₂alkyl, N,N-di-C₁₋₄alkylaminoC₀₋₂alkyl, N—C₁₋₄alkylcarbamoylC₀₋₂alkyl, N,N-di-C₁₋₄alkylaminocarbamoylC₀₋₂alkyl, N—C₁₋₆alkyaminocarbonylC₀₋₂alkyl, N,N—C₁₋₆alkyaminocarbonylC₀₋₂alkyl, C₁₋₆alkyl-S(O)ₙ-aminoC₀₋₂alkyl-, aryl-S(O)ₙ-aminoC₀₋₂alkyl-, C₁₋₃perfluoroalkyl-S(O)ₙ-aminoC₀₋₂alkyl-; C₁₋₆alkylamino-S(O)ₙ—C₀₋₂alkyl-, arylamino-S(O)ₙ—C₀₋₂alkyl-, C₁₋₃perfluoroalkylamino-S(O)ₙ—C₀₋₂alkyl-, C₁₋₆alkanoylamino-S(O), —C₀₋₂alkyl-; arylcarbonylamino-S(O)ₙ—C₀₋₂alkyl-, C₁₋₆alkyl-S(O)ₙ—C₀₋₂alkyl-, aryl-S(O)ₙ—C₀₋₂alkyl-, C₁₋₃perfluoroalkyl- or C₁₋₃perfluoroalkoxyC₀₋₂alkyl; wherein an amino group within R¹² is optionally substituted by C₁₋₄alkyl.

More preferably optional substituents on R⁸ are selected from: cyano, hydroxy, oxo, nitro, halo, trifluoromethyl, C₁₋₄alkyl, C₁₋₄alkoxy, C₁₋₄alkanoyl, R⁹OC(O)(CH₂)_{w}—, R⁹R¹⁰N(CH₂)_{w}—, R⁹R¹⁰NC(O)(CH₂)_{w}—, R⁹R¹⁰NC(O)(CH₂)_{w}—, R⁹R¹⁰NC(O)N(R⁹)(CH₂)_{w}—, R⁹OC(O)N(R⁹)(CH₂)_{w}—, or halo, wherein w is an integer between 0 and 4 and R⁹ and R¹⁰ are selected from: hydrogen, C₁₋₄alkyl, C₁₋₄alkylsulphonyl and C₃₋₇carbocyclyl.

Further preferably optional substituents on R⁸ are selected from: cyano, hydroxy, oxo, amino, N,N-diC₁₋₄alkyamino, N,N-diC₁₋₄alkyaminoC₁₋₄alkyl, N′-C₁₋₄alkylureido, N—C₁₋₄alkylsulphonylamino, N,N-di-C₁₋₄alkylsulphonylamino, nitro, halo, trifluoromethyl, C₁₋₄alkyl, C₁₋₄alkoxy, C₁₋₄alkanoyl, C₁₋₄alkoxycarbonylamino and C₃₋₇carbocyclylcarbonylamino.

More preferably optional substituents on R⁸ are selected from: cyano, hydroxy, oxo, methyl, ethyl, t-butyl, methoxy, acetyl, amino, N,N-dimethylamino, N′-isopropylureido, N′-cyclohexylureido, N-methylsulphonylamino, N,N-dimethylsulphonylamino, nitro, chloro, fluoro, trifluoromethyl, isopropoxycarbonylamino and cyclopentylcarbonylamino.

Further preferably optional substituents on R⁸ are selected from: hydroxy, methyl, ethyl, methoxy, fluoro, methylsulphonylamino, isopropylureido and isopropoxycarbonylamino. Most preferably optional substituents on R⁸ are selected from: methylsulphonylamino, isopropylureido and isopropoxycarbonylamino.

In a further embodiment of the invention optional substituents on R⁸ are selected from: C₁₋₄alkoxy, fluoro, C₁₋₄alkylsulphonylamino, C₁₋₄alkanoylamino, C₁₋₄alkylureido and C₁₋₄alkoxycarbonylamino.

In a further embodiment of the invention when R⁸ is phenyl then R⁸ is preferably substituted and when R⁸ is a heterocyclic ring R⁸ is preferably unsubstituted.
R⁵ is a group of formula (IIIa)-(III-o), for instance from (IIIa)-(IIIn) as defined above.

Preferably, in these groups, R¹⁶ and R^{16a} are independently selected from hydrogen and C₁₋₄alkyl. More preferably R¹⁶ and R^{16a} are independently selected from hydrogen, methyl and ethyl. Most preferably R¹⁶ and R^{16a} are both methyl.

Preferably R¹⁷ is hydrogen or methyl. Most preferably R¹⁷ is hydrogen.

Preferably R^{17a} is hydrogen or methyl. Most preferably R^{17a} is hydrogen.

Preferably A is selected from a direct bond, optionally substituted C₁₋₅alkylene, carbonyl or —C(O)—C(R^{d}R^{d})—, wherein R^{d} is independently selected from hydrogen and C₁₋₂alkyl, and wherein the optional substituents are independently selected from: hydroxy, hydroxyC₁₋₆alkyl, C₁₋₆alkyl, C₁₋₆alkoxy, C₁₋₄alkoxyC₁₋₄alkyl, aryl or arylC₁₋₆alkyl

Further preferably A is selected from C₁₋₅alkylene optionally substituted with C₁₋₄alkyl or C₁₋₄alkoxy, carbonyl or carbonylmethyl. Yet further preferably A is a direct bond or methylene. Most preferably A is methylene.

In a particular embodiment, B is a group of sub-formula (IV) or (V) as defined above.

In one embodiment, R¹¹ is selected from: hydrogen, optionally substituted C₁₋₆alkyl or N(R²³R²⁴), where R²³ and R²⁴ are as defined above.

Particular examples of R¹¹ is hydrogen or optionally substituted C₁₋₆alkyl where the optional substituents on the alkyl

groups are selected from R¹² and

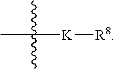

In a further embodiment, R¹¹ is a group NR²³R²⁴.

Suitably R²³ is selected from hydrogen, optionally substituted aryl, optionally substituted 3-10 membered heterocyclic ring or an optionally substituted C₁₋₈alkyl, wherein optional substituents are as defined above.

Suitably R²⁴ is selected from hydrogen or optionally substituted C₁₋₈alkyl,

When R¹³ or R⁴, but particularly R²³ is a C₁₋₆alkyl group, such as a C₁₋₆alkyl group, it is suitably optionally substituted 3 to 10 membered heterocyclic ring containing from 1 to 4 heteroatoms independently selected from O, N and S, the heterocyclic ring is preferably selected from pyridyl, thienyl, piperidinyl, imidazolyl, triazolyl, thiazolyl, pyrrolidinyl, piperazinyl, morpholinyl, imidazolinyl, benztriazolyl, benzimidazolyl, pyrimidinyl, pyrazinyl, pyridazinyl, oxazolyl, furanyl, pyrrolyl, 1,3-dioxolanyl, 2-azetinyl, each of which is optionally substituted, wherein the optional substituents are preferably selected from R¹² and

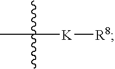

Further preferably the heterocyclic ring is a group of formula VI-a, VI-b, VI-c, VI-d, VI-e, VI-f, VI-g, VI-h, VI-i, VI-j or VI-k, wherein each group is optionally substituted by one or more groups selected from R¹² and

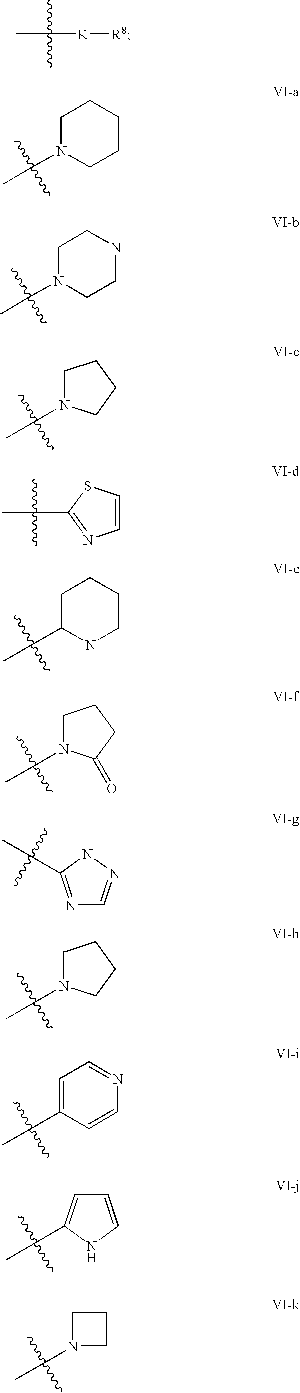

Most preferably the heterocyclic ring is a group of formula VI-a or VI-h, wherein each group is optionally substituted by one or more groups selected from R¹²

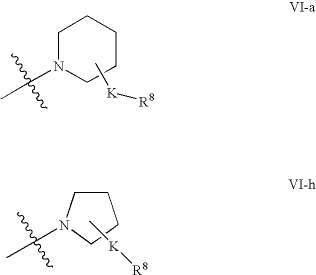

Preferably R²⁴ is optionally substituted C₁₋₆alkyl, or together with R²³ and the nitrogen atom to which they are attached, forms an optionally substituted heterocyclic ring of 3-10 atoms. Further preferably R²⁴ is selected from: methyl, ethyl or tert-butyl, or together with R²³ and the nitrogen atom to which they are attached, forms an optionally substituted heterocyclic ring of 3-10 atoms. Most preferably R²⁴ together with R²³ and the nitrogen atom to which they are attached, forms an optionally substituted heterocyclic ring of 3-10 atoms.

When N(R²³R²⁴) represents an optionally substituted 3- to 1 0-membered heterocyclic ring, for instance a 3-9 membered heterocyclic ring, N(R²³R²⁴) is preferably selected from a 5- or 6-membered monocyclic ring containing between 1 and 3 (preferably 1 or 2) heteroatoms independently selected from O, N and S, wherein the optional substituents are independently selected from R¹² and

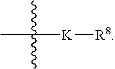

Further preferably N(R²³R²⁴) represents a 5- or 6-membered monocyclic ring containing between 1 and 3 (preferably 1 or 2) heteroatoms independently selected from O, N and S selected from pyrrolidinyl, thienyl, pyrazolidinyl, piperidinyl, morpholinyl, thiomorpholinyl piperazinyl, imidazole, or azetidinyl; wherein the optional substituents are independently selected from R¹² and

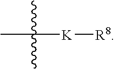

Further preferably the structure N(R²³R²⁴) is a heterocyclic ring selected from an optionally substituted group of formula, IV-a, IV-b, IV-c, IV-d and IV-e, wherein each group is optionally substituted by one or more groups selected from R¹² and 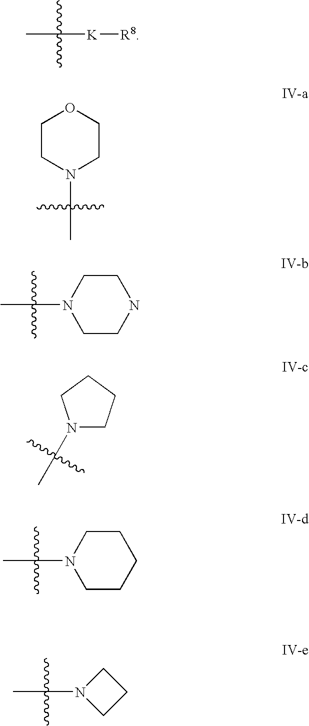
Further preferably the structure N(R²³R²⁴) is selected from a group of formula Va, Vb or Vc, wherein each group is optionally substituted by one or more groups selected from R¹². 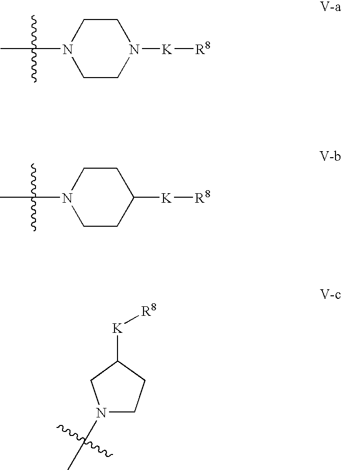
where K and R⁸ are as defined above.

Most preferably the structure N(R²³R²⁴) is a group of formula V-b or V-c, wherein each group is optionally substituted by one or more groups selected from R¹².

R¹¹ may also be a group NC(O)OR²⁵. R²⁵ is suitably optionally substituted C₁₋₆alkyl, and in particular unsubstituted C₁₋₄alkyl.

When B is a group (iii) listed above, it is suitably a group independently selected from: optionally substituted C₁₋₆alkylene, optionally substituted C₃₋₇cycloalkyl, optionally substituted C₃₋₆alkenylene, optionally substituted C₃₋₆alkynyl, (C₁₋₅alkyl)ₐₐ-S(Oₙ)—(C₁₋₅alkyl)_{bb}-, —(C₁₋₅alkyl)ₐₐ-O—(C₁₋₅alkyl)_{bb}-, —(C₁₋₅alkyl)ₐₐ-C(O)—(C₁₋₅alkyl)_{bb}- or (C₁₋₅alkyl)ₐₐ-N(R¹⁷)—(C₁₋₅alkyl)_{bb}, where aa, R¹⁷ and bb are as defined above.

Preferably B is selected from optionally substituted C₁₋₆alkylene, optionally substituted C₃₋₆alkenylene, —(C₁₋₅alkyl)ₐₐ-O—(C₁₋₅alkyl)_{bb}, —(C₁₋₅alkyl)ₐₐ-C(O)—(C₁₋₅alkyl)_{bb}-, —(CH₂)ₐₐ—C(O)N(R¹⁷)—(CH₂)_{sbb}, where optional substituents and R¹⁷ is as defined above, and aa and bb are independently 0 to 1, or the group 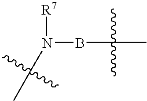
forms an optionally substituted C₄₋₇heterocyclic ring.

More preferably B is C₁₋₆alkylene, C₃₋₆alkenylene, —(C₁₋₅alkyl)ₐₐ-O—(C₁₋₅alkyl)_{bb}-, —(C₁₋₅alkyl)ₐₐ-C(O)—(C₁₋₅alkyl)_{bb}-, —(CH₂)ₛ₁—C(O)N(R¹⁷)—, or the group 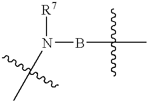
forms an optionally substituted saturated C₄₋₇heterocyclic ring, wherein aa and bb are independently 0 or 1 and wherein C₁₋₆alkylene is optionally substituted by hydroxy.

Further preferably B is unsubstituted C₁₋₆alkylene, C₃₋₆alkenylene —(C₁₋₅alkyl)ₐₐ-O—(C₁₋₅alkyl)_{bb}-, —(C₁₋₅alkyl)ₐₐ-C(O)— or the group 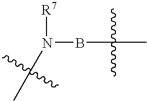
forms an optionally substituted saturated C₄₋₇heterocyclic ring selected from: azetidinyl, pyrrolidinyl, pyrazolinyl, pyrazolidinyl, imidazolinyl, imidazolidinyl, piperidinyl, piperazinyl, hexahydropyrimidinyl, hexahydropyridazinyl, hexahydrotriazinyl, tetraydrotriazinyl, dihydrotriazinyl, morpholinyl, thiomorpholinyl, thiazinanyl, thiazolidinyl, 1,5-dioxa-9-azaspiro[5.5]undecanyl or octahydropyrrolopyrrolyl, wherein the optional substituents are selected from: cyano, hydroxy, oxo, C₁₋₄alkyl, C₁₋₄alkoxy, C₁₋₄alkanoyl, R^{16c}OC(O)(CH₂)_{w}—, R^{16c}R^{16d}NC(O)(CH₂)_{w}— or halo, wherein w is an integer between 0 and 4 and R^{16e} and R^{16d} independently selected from the groups listed for R¹⁶ and R^{16a} above. Further preferably the optional substituents are selected from: cyano, hydroxy, oxo, C₁₋₄alkyl, C₁₋₄alkoxy and C₁₋₄alkanoyl, aa and bb are independently 0 or 1, and wherein C₁₋₆alkylene is optionally substituted by hydroxy.

Yet further preferably B is selected from: methylene, ethylene, propylene, propyl-2-ene, butylene, pentylene, 2-propenyl, propoxy, ethoxyethyl, methylcarbonyl or methylcarbonylamino.
or the group 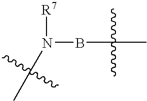
forms a C₄₋₇heterocyclic ring selected from: pyrrolidinyl, piperidinyl, or piperazinyl, wherein the optional substituents are selected from oxo.

Most preferably B is selected from ethylene or butylene.

In another embodiment of the invention preferably B is selected from optionally substituted C₁₋₆alkylene or the group 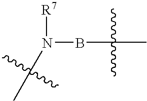
forms a C₅₋₇heterocyclic ring. Preferably B is selected from unsubstituted C₁₋₆alkylene or the group 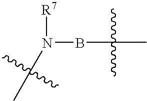
forms a saturated C₅₋₇heterocyclic ring. Most preferably B is selected from methylene, ethylene, propylene, butylene or the group 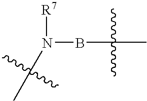
forms a saturated C₅₋₇heterocyclic ring selected from piperidinyl or piperazinyl.

Preferably M is —CH₂—CH₂—.

When R³ is selected from a group of Formula (IIc) or Formula (IId) then the group 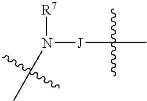
preferably forms an optionally substituted heterocyclic ring containing 4-7 carbons atoms, wherein the optional substituents are selected from 1 or 2 substituents independently selected from R¹² and R¹³.

More Preferably the group 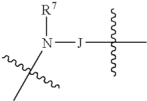
forms an optionally substituted saturated C₄₋₇heterocyclic ring wherein the optional substituents are selected from 1 or 2 substituents independently selected from R¹² and R¹³.

Further Preferably the Group 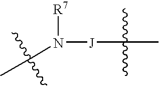
forms an optionally substituted saturated C₄₋₇heterocyclic ring selected from: azetidinyl, pyrrolidinyl, pyrazolinyl, pyrazolidinyl, imidazolinyl, imidazolidinyl, piperidinyl, piperazinyl, hexahydropyrimidinyl, hexahydropyridazinyl, hexahydrotriazinyl, tetraydrotriazinyl, dihydrotriazinyl, morpholinyl, thiomorpholinyl, thiazinanyl, thiazolidinyl or octahydropyrrolopyrrolyl, wherein the optional substituents are selected from oxo, C₁₋₄alkyl and C₁₋₄alkoxy.

Further Preferably the Group 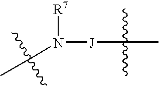
forms an optionally substituted saturated C₄₋₇heterocyclic ring selected from: pyrrolidinyl, piperidinyl or piperazinyl, wherein the optional substituents are selected from C₁₋₄alkoxy, oxo and C₁₋₄alkyl.

Most Preferably the Group 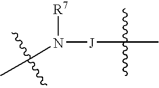
forms an optionally substituted saturated C₄₋₇heterocyclic ring selected from: piperazinyl or piperidinyl.

Preferably K is selected from: a direct bond, —(CH₂)ₛ—, —(CH₂)ₛ—O—(CH₂)ₛ—, —(CH₂)ₛ—C(O)—(CH₂)ₛ—, —(CH₂)ₛ—N(R^{17a})—(CH₂)ₛ—, —(CH₂), —C(O)N(R^{17a})—(CH₂)ₛ—, —(CH₂)ₛ—N(R^{17a})C(O)—(CH₂)ₛ—, —(CH₂)ₛ—S(O)₂N(R^{17a})—(CH₂)ₛ—, or —(CH₂)ₛ—NHS(O)₂—(CH₂)ₛ—, wherein s is independently selected from 0, 1, 2, 3 or 4, R^{17a} is selected from hydrogen or C₁₋₄alkyl (preferably hydrogen) and the —(CH₂)ₛ— group is optionally substituted by hydroxy or C₁₋₄alkyl.

More preferably K is selected from: a direct bond, —(CH₂)ₛ—, —(CH₂), —O—(CH₂)ₛ—, —(CH₂)ₛ—C(O)—, —C(O)—(CH₂)ₛ—, —(CH₂), —N(R^{17a})—, —(CH₂)ₛ—C(O)N(R^{17a})—, —(CH₂)ₛ—N(R^{17a})C(O)—(CH₂)ₛ—, —(CH₂)ₛ—S(O)₂N(R^{17a})— or —(CH₂)ₛ—NHS(O)₂—, wherein s is independently selected from 0, 1, 2, 3 or 4, R^{17a} is selected from hydrogen or C₁₋₄alkyl (preferably hydrogen or methyl) and the —(CH₂)ₛ— group is optionally substituted by hydroxy or C₁₋₄alkyl.

More preferably K is selected from: a direct bond, methylene, ethylene, propylene, butylene, oxy, 2-hydroxypropylene, carbonyl, methylcarbonyl, ethylcarbonyl, (methyl)methylcarbonyl, (ethyl)methylcarbonyl, carbonylmethylene, carbonylethylene, ethoxyethylene, amino, 2-hydroxypropylamino, carbonylamino, methylcarbonylamino, N-methyl-methylcarbonylamino, aminocarbonyl, methylaminocarbonyl, methylaminocarbonylmethyl, propylsulphonylamino or methylaminosulphonyl.

Further preferably K is selected from: a direct bond, methylene, ethylene, propylene, butylene carbonyl, methylcarbonyl or N-methylmethylcarbonylamino.

Further preferably K is selected from: a direct bond, methyl, carbonyl and methylcarbonyl.

When J is a group of the formula: —(CH₂)ₛ-L-(CH₂)ₛ— or —(CH₂), —C(O)—(CH₂)ₛ-L-(CH₂)ₛ—, at least one and suitably all s groups are 0.

Groups L are optionally substituted aryl or optionally substituted heterocyclyl groups. Suitable optional substituents for groups L include those listed above for R¹². Preferably L is unsubstituted other than by the adjacent —(CH₂)ₛ— groups.

In particular, L is an optionally substituted heterocyclic group as defined above. In particular it is a 4-12 membered, preferably 5-10 membered saturated or partially saturated mono or bicyclic ring includes at least one nitrogen atom. Preferably the nitrogen atom is linked to an adjacent —(CH₂)ₛ group. Examples of saturated or partially saturated heterocyclic rings include azethidinyl, pyrrolinyl, pyrrolidinyl, morpholinyl, piperidinyl, piperazinyl, dihydropyridinyl, benzodioxyl and dihydropyrimidinyl. A particularly preferred group L is azetidinyl.

According to a further aspect of the invention there is provided a compound of Formula (Ia) 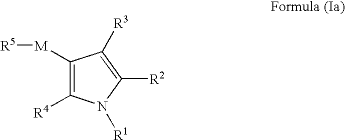
wherein:

R³ is selected from a group of Formula (IIa) or Formula (IIb):

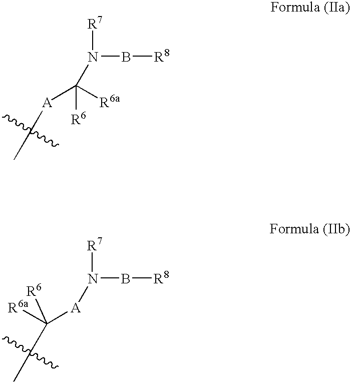

R⁷ is selected from: hydrogen or C₁₋₆alkyl;

B is a group of Formula (IV)

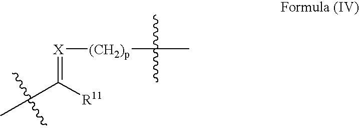

and A, M, R¹, R², R⁴, R⁵R⁶, R^{6a}, R⁸, and R¹¹ are as defined above for a compound of Formula (I)

or a salt, solvate or pro-drug thereof.

According to a further aspect of the invention there is provided a compound of Formula (Ia) wherein:

X is N;

R⁸ is —C(O)O—R^{b}, wherein R^{b} is as defined above;

or a salt, solvate or pro-drug thereof.

According to a further aspect of the invention there is provided a compound of Formula (Ib) 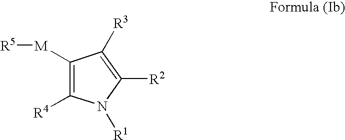
wherein:

R³ is selected from a group of Formula (IIa) or Formula (IIb):

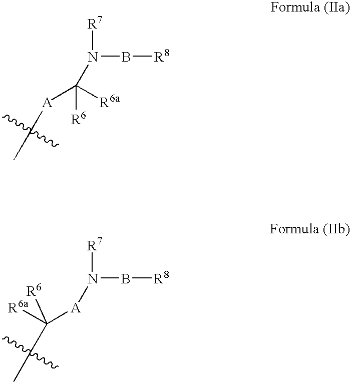

wherein
the group 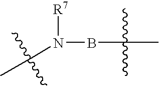
   together forms an optionally substituted heterocyclic ring containing 4-7 carbons atoms, wherein the optional substituents are selected from 1 or 2 substituents independently selected from R¹² and R¹³;
and A, M, B, R¹, R², R⁴, R⁵, R⁶, R^{6a}, R⁸, R¹² and R¹³ are as defined above for a compound of Formula (I)
   or a salt, solvate or pro-drug thereof.

According to a further aspect of the invention there is provided a compound of Formula (Ic) 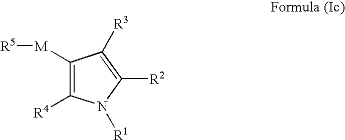
wherein:

R³ is selected from a group of Formula (IIc) or Formula (IId):

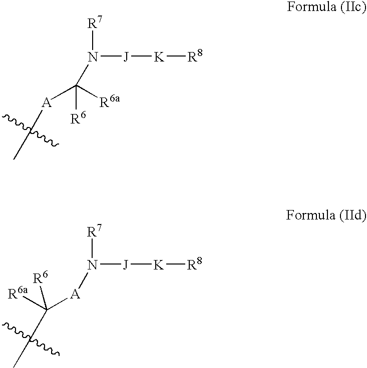

wherein
the group 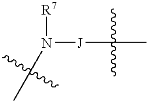
   together forms an optionally substituted heterocyclic ring containing 4-7 carbons atoms, wherein the optional substituents are selected from 1 or 2 substituents independently selected from R¹² and R¹³;
and A, M, J, R¹, R², R⁴, R⁵ R⁶, R^{6a}, R⁸, and R¹² and R¹³ are as defined above for a compound of Formula (I)
   or a salt, solvate or pro-drug thereof.

According to a further aspect of the invention there is provided a compound of Formula (Ic), wherein:
K is —(CH₂)ₛ₁—C(O)—(CH₂)ₛ₂— or —(CH₂)ₛ₁—;
R⁸ is selected from: C₃₋₇cycloalkyl, aryl or heterocyclyl each of which is optionally substituted by one or substituents independently selected from R¹² or R¹³; and
s1 and s2 are as defined above;
   or a salt, solvate or pro-drug thereof.

According to a further aspect of the invention there is provided a compound of Formula (Id) 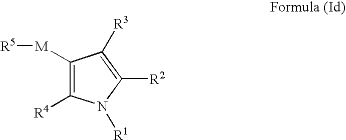
wherein:
R³ is selected from a group of Formula (IIc) or Formula (IId):

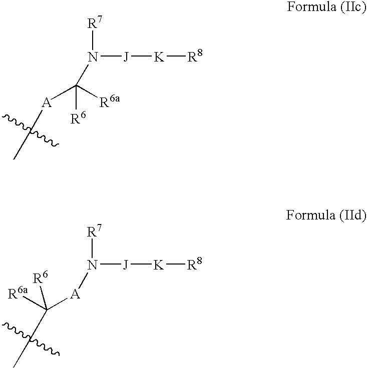

wherein
J is a group of the formula: —(CH₂)ₛ-L-(CH₂)ₛ— or
—(CH₂)ₛ—C(O)—(CH₂)ₛ-L-(CH₂)ₛ— wherein when s is greater than 0, the alkylene group is optionally substituted by 1 to 2 group selected from R¹²,
and A, K, L, M, R¹, R², R⁴, R⁵R⁶, R^{6a}, R⁸, and R¹² are as defined above for a compound of Formula (I)
   or a salt, solvate or pro-drug thereof.

According to a further aspect of the invention there is provided a compound of Formula (Ie) 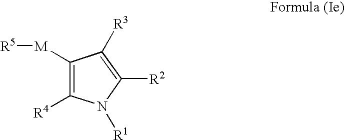
wherein:
R³ is selected from a group of Formula (IIa) or Formula (IIb): 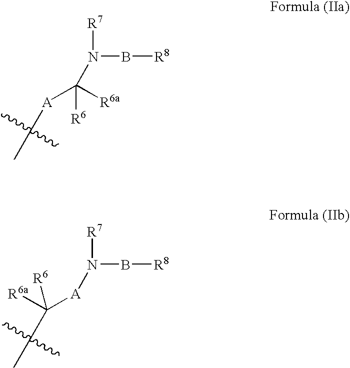
B is optionally substituted C₁₋₆alkylene, wherein the optional substituents are independently selected from R¹²;
R⁷ is selected from: hydrogen or C₁₋₆alkyl;
R⁸ is selected from: C₃₋₇cycloalkyl, aryl or heterocyclyl each of which is optionally substituted by one or substituents independently selected from R¹² or R¹³;
and A, M, R¹, R², R⁴, R⁵ R⁶, R^{6a} and R¹¹ are as defined above for a compound of Formula (I);
   or a salt, solvate or pro-drug thereof.

According to a further aspect of the invention there is provided a compound of Formula (Ie)

wherein

R⁸ is selected from: aryl optionally substituted by one or substituents independently selected from R¹² or R¹³, preferably substituted R¹²;
   or a salt, solvate or pro-drug thereof.

A further preferred group of compounds of the invention comprises a compound of Formula (If):

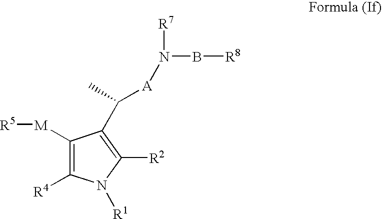

wherein R¹, R², R⁵; R⁷, R⁸, A, B and M are as defined above

or salt, solvate or pro-drug thereof.

A further preferred group of compounds of the invention comprises a compound of formula (Ia), (Ib), (Ic), (Id), (Ie) or (If), wherein:
R⁵ is selected from one of the following groups:

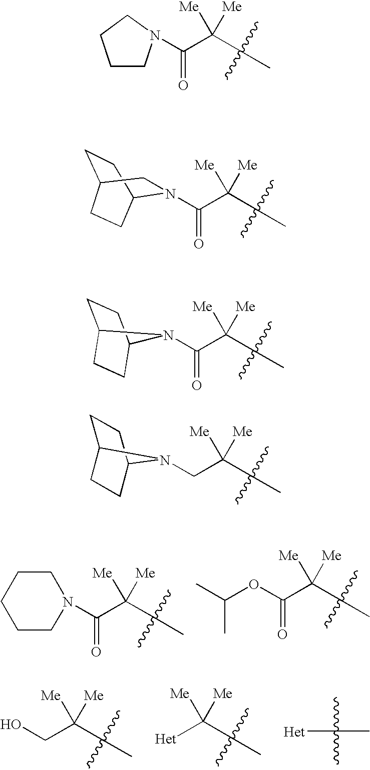

wherein Me represents methyl and het is as defined above.

or salt, solvate or pro-drug thereof.

A further preferred group of compounds of the invention comprises a compound of Formula (Ia), (Ib), (Ic), (Id), (Ie) or (If), wherein:
R² represents 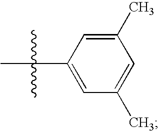
   or salt, solvate or pro-drug thereof.

A further preferred group of compounds of the invention comprises a compound of Formula (Ia), (Ib), (Ic), (Id), (Ie) or (If), wherein:
R² represents 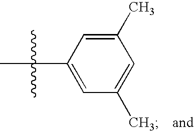
R⁵ is selected from one of the following groups:

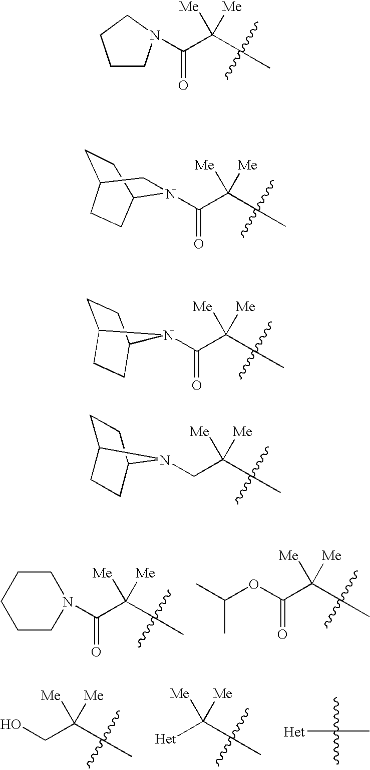

wherein Me represents methyl and het is as defined above.

or salt, solvate or pro-drug thereof.

Particularly preferred compounds according to the present invention are wherein the compound is selected from:
3-[3,3-Dimethyl-4-oxo-4-(azabicyclo[2.2.1]heptan-7-yl)butyl]-4-[1S-methyl-2-(N′-isopropoxycarbonyl-3-pyrid-4-yl-pyrrolidin-1-ylcarboximidamido)ethyl]-5-(3,5-dimethylphenyl)-1H-pyrrole;
3-[3,3-Dimethyl-4-oxo-4-(azabicyclo[2.2.1]heptan-7-yl)butyl]-4-[2-{4-(pyrrolidin-1-ylcarbonyl)piperazin-1-yl}ethyl]-5-(3,5-dimethylphenyl)-1H-pyrrole;
3-[3,3-Dimethyl-4-oxo-4-(azabicyclo[2.2.1]heptan-7-yl)butyl]-4-[2-{4-(4-hydroxypiperidin-1-ylcarbonyl)piperidin-1-yl}ethyl]-5-(3,5-dimethylphenyl)-1H-pyrrole;
3-[3,3-Dimethyl-4-oxo-4-(azabicyclo[2.2.1]heptan-7-yl)butyl]-4-[2-{4-(1,1-dioxo-isothiazolidin-2-ylcarbonyl)-4-methoxy-piperidin-1-yl}ethyl]-5-(3,5-dimethylphenyl)-1H-pyrrole;
3-[3,3-Dimethyl-4-oxo-4-(azabicyclo[2.2.1]heptan-7-yl)butyl]-4-[1S-methyl-2-(2-{4-N-isopropylureidophenyl}ethylamino)ethyl]-5-(3,5-dimethylphenyl)-1H-pyrrole;
   or a salt, pro-drug or solvate thereof.

More particularly preferred compounds according to the present invention are wherein the compound is selected from:
3-[3,3-Dimethyl-4-oxo-4-(azabicyclo[2.2.1]heptan-7-yl)butyl]-4-[1S-methyl-2-(N′-isopropoxycarbonyl-3-pyrid-4-yl-pyrrolidin-1-ylcarboximidamido) ethyl]-5-(3,5-dimethylphenyl)-1H-pyrrole;
3-[3,3-Dimethyl-4-oxo-4-(azabicyclo[2.2.1]heptan-7-yl)butyl]-4-[2-{4-(4-hydroxypiperidin-1-ylcarbonyl)piperidin-1-yl}ethyl]-5-(3,5-dimethylphenyl)-1H -pyrrole;
3-[3,3-Dimethyl-4-oxo-4-(azabicyclo[2.2.1]heptan-7-yl)butyl]-4-[2-{4-(1,1-dioxo-isothiazolidin-2-ylcarbonyl)-4-methoxy-piperidin-1-yl}ethyl]-5-(3,5-dimethylphenyl)-1H-pyrrole;
3-[3,3-Dimethyl-4-oxo-4-(azabicyclo[2.2.1]heptan-7-yl)butyl]-4-[1S-methyl-2-(2-{4-N-isopropylureidophenyl}ethylamino)ethyl]-5-(3,5-dimethylphenyl)-1H-pyrrole;
   or a salt, pro-drug or solvate thereof.

The most preferred compound according to the present invention is:
3-[3,3-Dimethyl-4-oxo-4-(azabicyclo[2.2.1]heptan-7-yl)butyl]-4-[1S-methyl-2-(2-{4-N-isopropylureidophenyl}ethylamino)ethyl]-5-(3,5-dimethylphenyl)-1H-pyrrole;
   or a salt, pro-drug or solvate thereof.

The compounds of Formula (I) may be administered in the form of a pro-drug which is broken down in the human or animal body to give a compound of the Formula (I). Examples of pro-drugs include in-vivo hydrolysable esters of a compound of the Formula (I). Various forms of pro-drugs are known in the art. For examples of such pro-drug derivatives, see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H.

Bundgaard, Chapter 5 “Design and Application of Prodrugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77, 285 (1988); and
e) N. Kakeya, et al., Chem Pharm Bull, 32, 692 (1984).

An in-vivo hydrolysable ester of a compound of the Formula (I) containing a carboxy or a hydroxy group is, for example, a pharmaceutically-acceptable ester which is hydrolysed in the human or animal body to produce the parent acid or alcohol. Suitable pharmaceutically-acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters, for example 5-methyl-1,3-dioxolen-2-onylmethyl; and C₁₋₆alkoxycarbonyloxyethyl esters.

An in-vivo hydrolysable ester of a compound of the Formula (I) containing a hydroxy group includes inorganic esters such as phosphate esters (including phosphoramidic cyclic esters) and α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxy-methoxy. A selection of in-vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl.

A suitable pharmaceutically-acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically-acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

The compounds of Formula (I) can be prepared by a process comprising a step selected from (a) to (h) as follows, these processes are provided as a further feature of the invention:—
(a) Reaction of a compound of formula XXXII with a compound of formula H—R³′ to form a compound of Formula (I), 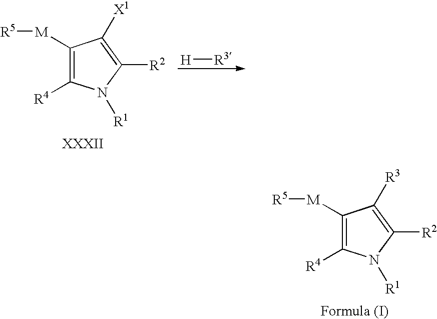
   wherein X¹ is selected from: 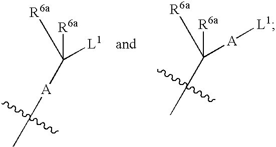
   L¹ is a displaceable group; and H—R^{e} is selected from: 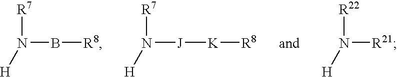
(b) Reaction of a compound of formula XXXIII with a compound of formula L²-R³″ to form a compound of Formula (I), 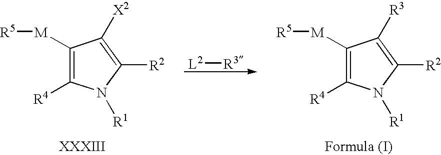
   wherein X² is selected from: 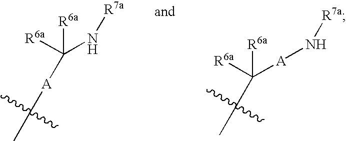
      L² is a displaceable group and R^{7a} is selected from the definition of R⁷ or R²² above, and L²-R³″ is selected from: L²-B—R⁸, L²-J-K—R⁸ and L²-R²¹
(c) For compounds of Formula (I) wherein R⁷ is other than part of a heterocyclic ring or hydrogen, reaction of a compound of Formula (d) wherein R³ is a group of Formula (IIa), (IIb), (IIc) or (IId) and R⁷ is hydrogen with a group of formula L³-R^{7a}, wherein R^{7a} is as defined above for R⁷ with the exclusion of hydrogen and L³ is a displaceable group;
(d) For compounds of Formula (I) wherein R⁴ is hydrogen, reduction of a thienopyrrole of Formula XXXVIII to a compound of Formula (I) 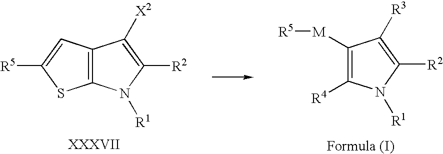
(e) For compounds of Formula (I) wherein R³ is a group of Formula (IIc) or (IId) and the group 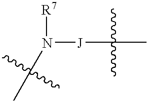
   together forms an optionally substituted nitrogen-containing heterocyclic ring containing 4-7 carbons atoms, reaction of a compound of Formula XXXIVa or XXXIVb, with a compound of Formula L⁶-K—R⁸, wherein L⁶ is a displaceable group 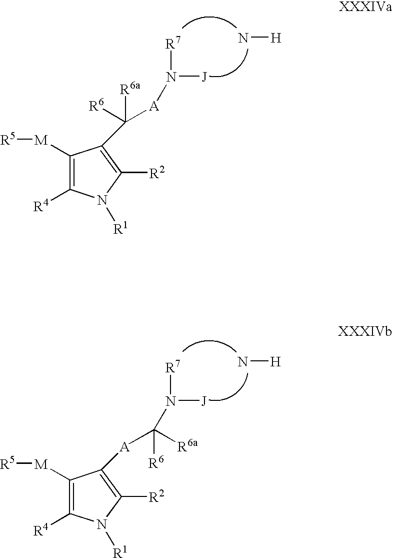
(f) For compounds of Formula (I) wherein R³ is a group of Formula (IIc) or (IId), reaction of a compound of Formula XXXVa or XXXVb, with a compound of Formula L⁷-K″—R⁸, wherein L⁷ is a displaceable group, and wherein the groups K′ and K″ comprise groups which when reacted together form K, 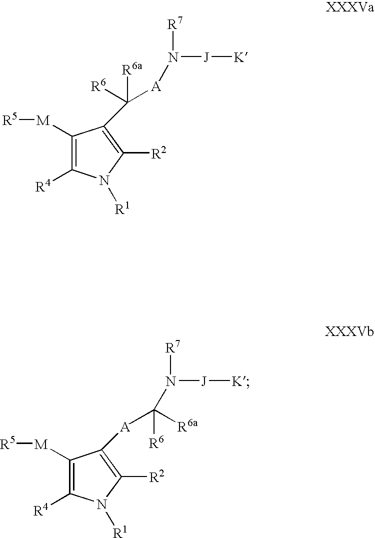
(g) reaction of a compound of Formula XXXVI with an electrophilic compound of the formula L⁸-R³, wherein L⁸ is a displaceable group 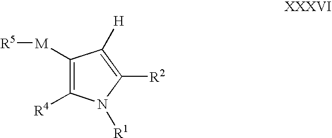
(h) reaction of a compound of Formula XXXIX with an appropriate electrophilic reagent to give a compounds of Formula (I) 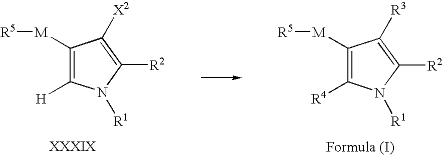
   and thereafter if necessary, carrying out one or more of the following steps:
   i) converting a compound of the Formula (I) into another compound of the Formula (I);
   ii) removing any protecting groups;
   iii) forming a salt, pro-drug or solvate.

Specific reaction conditions for the above reactions are as follows:

Process a) Compounds of formula XXXII and H—R⁵′ can be coupled together in the presence of an organic base (such as DIPEA [di-isopropylethylamine]) or an inorganic base (such as potassium carbonate) base, in a suitable solvent such as DMA or DMF, at a temperature from room temperature and 120° C. Suitable displaceable groups include: a halide, such as chloro, or a methane sulphonate or toluene sulphonate;

Process b) Compounds of XXXIII and L²-R³″ can be coupled together in the presence of an organic base (such as DIPEA) or an inorganic base (such as potassium carbonate), in a suitable solvent such as DMA or DMF, at a temperature from room temperature to 120° C. Suitable displaceable groups include: a halide, such as chloro, or a methane sulphonate or toluene sulphonate,

alternatively if L² is a hydroxy group then the L²-R³″; can be reacted with a compound of formula XXXIII under Mitsunobu reaction conditions;

Process c, and e) Reaction conditions to facilitate these reactions can be using (i) alkylation reaction conditions or (ii) acylation reaction conditions: Examples of said conditions include:

(i) alkylation reaction conditions—the presence of an organic base (such as DIPEA) or an inorganic base (such as potassium carbonate), in a suitable solvent such as DMF, DMA, DCM, at a temperature from room temperature to 120° C. Suitable displaceable groups include: a halide, such as chloro, methane sulphonate or toluene sulphonate;
(ii) acylation reaction conditions—presence of organic base, such as triethylamine, temperature 0° C. to 50-60° C. in a suitable solvent such as DCM. Suitable displaceable groups include an acylchloride or an acid anhydride,
   Process d) treatment of a compound of Formula XXXVIII with Raney-Nickel under hydrogen in a suitable solvent such as ethanol or methanol at a temperature between room temperature and the boiling point of the solvent.
   Process f) The skilled man would be familiar with a variety of reaction conditions and values for K′ and K″, which when reacted together would form the group K, examples of said conditions and values for K′ and K″ include:
(i.) For compounds of Formula (I) where K is —(CH₂)ₛ₁—N(R¹⁷)C(O)—(CH₂)ₛ₂— these can be prepared by reacting a compound where K′ is —(CH₂)ₛ₁—N(R¹⁷)H with a carboxylic acid for formula HOOC—(CH₂)ₛ₂—R⁸ to form the amide. Coupling of amino groups with carboxylic acids are well known in the art and can be facilitated by a number of chemical reactions using an appropriate coupling reagent. For example a carbodiimide coupling reaction can be performed with EDCl in the presence of DMAP in a suitable solvent such as DCM, chloroform or DMF at room temperature;
(ii.) For compounds of Formula (I) where K is —(CH₂)ₛ₁—C(O)N(R¹⁷)—(CH₂)ₛ₂ these can be prepared by reacting a compound where K′ is —(CH₂)ₛ₁—COOH with an amine of the HN(R¹⁷)—(CH₂)ₛ₂—R⁸ to form the amide. Methodology is identical to processes described in (i) above in this section;
(iii.) For compounds of Formula (I) where K is —(CH₂)ₛ₁— N(R¹⁷)C(O)O—(CH₂)ₛ₂— these can be prepared by reacting a compound where K′ is —(CH₂)ₛ₁—N(R¹⁷)H with a chloroformate of formula ClC(O)O—(CH₂)ₛ₂—R⁸ in a suitable solvent, such as DCM or chloroform, in the presence of a base, such as N-methylmorpholine, pyridine or triethylamine, at a temperature between −10° C. and 0° C.;
(iv.) For compounds of Formula (I) where K is —(CH₂)ₛ₁—OC(O)N(R¹⁷)—(CH₂)ₛ₂— these can be prepared by reacting a compound where K′ is —(CH₂)ₛ₁—OC(O)Cl with a compound of formula HN(R¹⁷)—(CH₂)ₛ₂—R⁸. Methodology is identical to processes described in (iii) above in this section;
(v.) For compounds of Formula (J) where K is —(CH₂)ₛ₁—N(R¹⁷)S(O₂)—(CH₂)ₛ₂— these can be prepared by reacting a compound where K′ is —(CH₂)ₛ₁—N(R¹⁷)H with a sulphonyl chloride of formula ClS(O₂)—(CH₂)ₛ₂—R⁸ in the presence of a base, such as triethylamine or pyridine, in a suitable solvent such as chloroform or DCM at a temperature between 0° C. and room temperature;
(vi.) For compounds of Formula (I) where K is —(CH₂)ₛ₁—S(O₂)N(R¹⁷)—(CH₂)ₛ₂— these can be prepared by reacting a compound where K′ is —(CH₂)ₛ₁—S(O₂)Cl with a compound of HN(R¹⁷)—(CH₂)ₛ₂—R⁸. Methodology is identical to processes described in (v) above in this section
(vii.) For compounds of Formula (I) where K is —(CH₂)ₛ₁— N(R¹⁷)—(CH₂)ₛ₂— these can be prepared by reacting a compound where K′ is —(CH₂)ₛ₁-L¹¹ with a compound of formula HN(R¹⁷)—(CH₂)ₛ₂—R⁸, wherein L¹¹ is a displaceable group. This reaction can be performed in the presence of an organic base (such as DIPEA) or an inorganic base (such as potassium carbonate), in a suitable solvent such as DMA or DMF, at a temperature from room temperature to 120° C. Suitable displaceable groups include: a halide, such as chloro, or a methane sulphonate or toluene sulphonate. Compounds can also be prepared by reacting a compound wherein K′ is —(CH₂)ₛ₁—N(R¹⁷)H with a compound of formula L¹¹-(CH₂)ₛ₂—R⁸, under identical conditions.
(viii.) For compounds of Formula (I) where K is —(CH₂)ₛ₁—O(CH₂)ₛ₂— these can be prepared by reacting a compound where K′ is —(CH₂)ₛ₁—OH with a compound of formula L¹²-(CH₂)ₛ₂—R⁸, wherein L¹² is a displaceable group. This reaction can be performed in the presence of an organic base (such as potassium t-butoxide) or an inorganic base (such as sodium hydride), in a suitable solvent such as DMA or DMF, at a temperature from room temperature and 120° C. Suitable displaceable groups include: a halide, such as bromo, or a methane sulphonate or toluene sulphonate. Compounds can also be prepared by reacting a compound wherein K′ is —(CH₂)ₛ₁-L¹² with a compound of formula HO—(CH₂)ₛ₂—R⁸, under identical conditions.
(ix.) For compounds of Formula (J) where K is —(CH₂)ₛ₁—C(O)—(CH₂)ₛ₂— these can be prepared by reacting a compound where K′ is —(CH₂)ₛ₁—C(O)-L¹³ with a Grignard reagent of formula BrMg(CH₂)ₛ₂—R⁸, wherein L¹³ is a displaceable group. This reaction can be performed in a non-polar solvent such as THF or diethylether at a temperature between room temperature and the boiling point of the solvent. Suitable displaceable groups include: a halide, such as bromo, or a methane sulphonate or toluene sulphonate. Compounds can also be prepared by reacting a compound wherein K′ is —(CH₂)ₛ₁—MgBr with a compound of formula L¹³-C(O)—(CH₂)ₛ₂—R⁸, under identical conditions.

Process g) reaction of a compound of Formula XXXVI with a compound of the formula L⁸-R³, can be performed under Friedel Craft conditions, for example in the presence of diethylaluminum chloride in a suitable solvent, such as DCM, in an inert atmosphere such as nitrogen, at a temperature between room temperature and the boiling point of the solvent or under Mannich conditions, for example, formaldehyde and a primary or secondary amine in acetic acid, in an inert atmosphere such as nitrogen at a temperature between room temperature and 100° C.

Process h) reaction of a compound of Formula XXXIX with an appropriate electrophilic reagent can be used to add an R⁴ group. For example, when R⁴ is a halogen, such as chlorine, an electrophilic reagent such as N-chlorosuccinimide in a suitable solvent, such as THF, at a temperature between room temperature and the boiling point of the solvent can be used, or when R⁴ is alkyl, such as ethyl, an electrophilic reagent such as an appropriate alkyl halide, such as ethyl iodide, can be used under Friedel Craft conditions, for example in the presence of diethylaluminum chloride in a suitable solvent, such as CH₂Cl₂, in an inert atmosphere such as nitrogen, at a temperature between room temperature and the boiling point of the solvent.

It will be appreciated by those skilled in the art that in the processes of the present invention certain functional groups such as hydroxyl or amino groups in the starting reagents or intermediate compounds may need to be protected by protecting groups. Thus, the preparation of the compounds of Formula (I) may involve, at an appropriate stage, the addition and subsequent removal of one or more protecting groups.

The protection and de-protection of functional groups is described in ‘Protective Groups in Organic Chemistry’, edited by J. W. F. McOmie, Plenum Press (1973) and ‘Protective Groups in Organic Synthesis’, 2nd edition, T. W. Greene and P. G. M. Wuts, Wiley-Interscience (1991).

A suitable protecting group for an amino or alkylamino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The de-protection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium-on-carbon.

### EXPERIMENTAL

General Reaction Schemes

In the following schemes wherein Ri, Rii and Riii represent optional substituents on the phenyl ring which are optionally protected as necessary and R represents a protecting group, group C has been depicted as substituted phenyl for illustration purposes only. Other definitions of C are also appropriate.

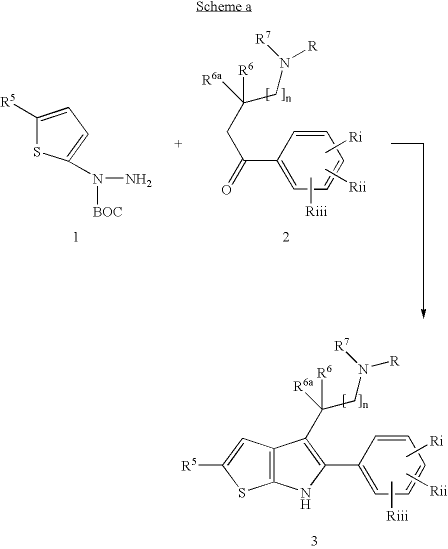

Thienopyrroles, such as 3 can be synthesised by the classic Fisher thienopyrrole synthesis reaction by the condensation of a hydrazine-HCl 1 and a ketone 2, bearing hydrogen atoms cc to the carbonyl (Scheme a). Treatment of these reactants in a suitable solvent, such as acetic acid, ethanol, sec-butanol, toluene, in the presence of an acid, such as sulphuric, hydrochloric, polyphosphoric and/or a Lewis acid, for example, boron trifluoride, zinc chloride, magnesium bromide, at elevated temperatures (for example 100° C.), gives the desired product. R represents a protecting group, eg tert-butylcarbamate or phthalimide.

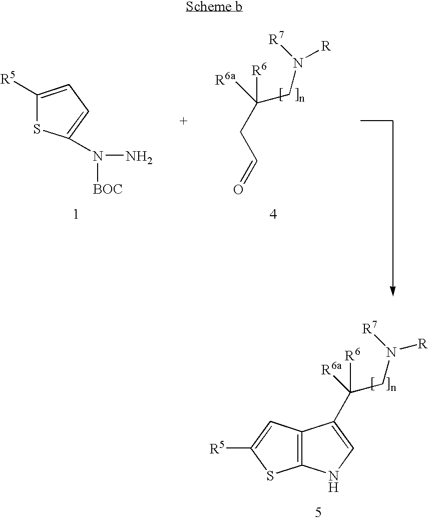

Thienopyrroles, such as represented in structure 5, can also be made using aldehydes 4, bearing hydrogen atoms a to the carbonyl, by cyclization using the conditions above. In this case the substituent at the 2-position must be added later (see scheme d).

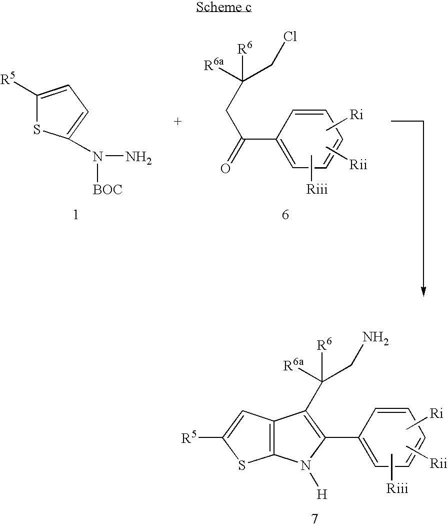

Thienopyrrole may also be synthesised utilising the Granburg reaction, wherein a hydrazine 1 is mixed with ketone 6, bearing a chlorine atom γ to the carbonyl, and heated in a suitable solvent such as ethanol, sec-butanol, toluene at a temperature between 50° C. and 120° C. (Scheme c).

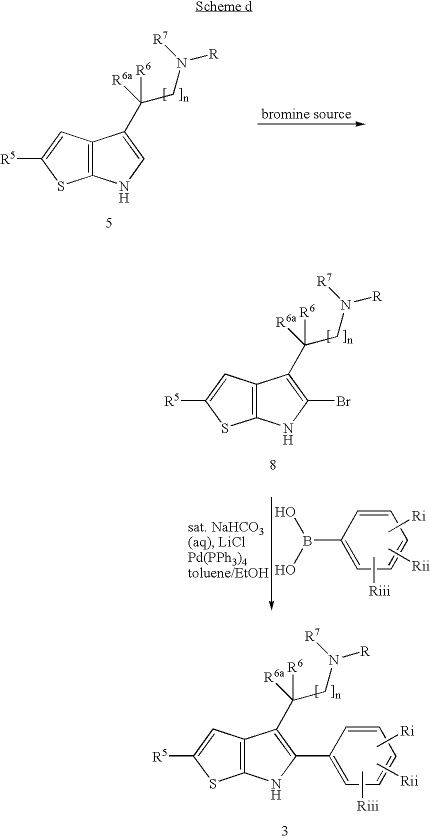

The thienopyrrole 5 can be treated with a ‘bromine source’, such as molecular bromide, pyridinium tribromide, pyrrolidone hydrobromide or polymer supported reagent equivalents, in an inert solvent such as chloroform, methylene chloride at −10° C. to 25° C. to yield the 2-bromo compound 8 (Scheme d). Reaction under Suzuki conditions with a palladium(0) catalyst, a weak base such as aqueous sodium carbonate or saturated sodium hydrogen carbonate and the like, and a substituted aryl boronic acid from commercial sources or prepared (as described in: Gronowitz, S.; Hornfeldt, A.-B.; Yang, Y.-H Chem. Sci. 1986, 26, 311-314), in an inert solvent such as toluene, benzene, dioxane, THF, DMF and the like, with heating between 25° C. and 100° C., preferably 80° C., for a period of 1-12 hours, to give the desired compound 3.

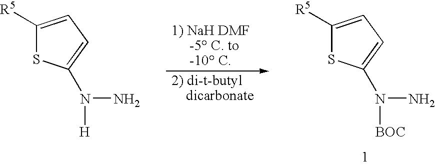

The thiophene 1 can be synthesised by reaction of a hydrazine under the preferred conditions of sodium hydride in DMF at a temperature between −10° C. and −5° C., followed by reaction with di-tert-butyldicarbonate in TIE under reflux.

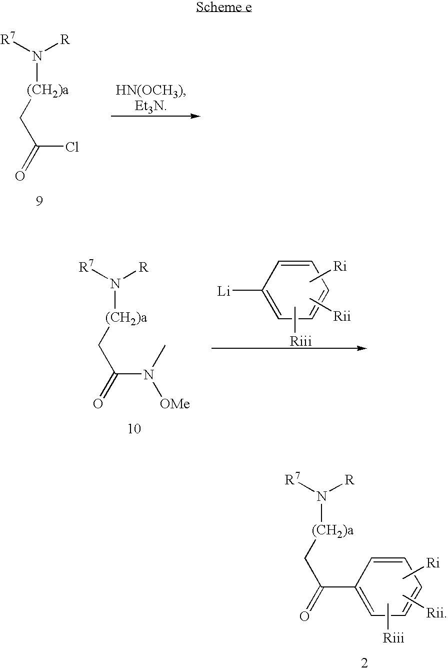

Substituted ketones 2 can be prepared, as outlined in Scheme e starting from appropriate acid chlorides such as 9. Treatment of the acid chloride with N,N-dimethylhydroxylamine hydrochloride in the presence of an amine base such as triethylamine, and a suitable solvent such as methylene chloride at a temperature of −10° C. to 25° C., yields the amide 10. Further reaction with a substituted aryl organolithium (prepared essentially as described in Wakefield B, J.; Organolithium Methods Academic Press Limited, 1988, pp. 27-29 and references therein) in an inert solvent such as tetrahydrofuran, diethyl ether, benzene, toluene or mixture thereof and the like, at a temperature between −100° C. and 0° C. then quenching of the reaction mixture with a mineral acid such as hydrochloric acid, yields the aryl ketone 2.

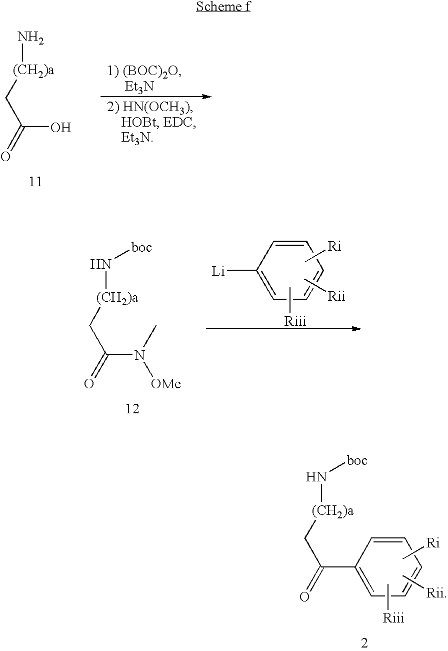

Commencing with a readily available amino acid with a suitable chain length [a]11, the nitrogen atom can be brought in at the beginning of the synthesis by the route shown in Scheme f. Protection of the amine group of 11 with a tert-butylcarbamate group is achieved by condensation with di-tert-butyl di-carbonate in the presence of an amine base, for example triethylamine, in an inert solvent such as methylene chloride, chloroform, benzene, toluene, tetrahydrofuran and mixtures thereof and the like, at a temperature of −10° C. to 25° C. Coupling of the acid product with N,N-dimethylhydroxylamine in the presence of a coupling reagent 1-(3-dimethylaminopropyl)-3-ethylcarbodiimide hydrochloride (EDC) or 1,3-dicyclohexylcarbodiimide (DCC) or the like, with or without 1-hydroxybenzotriazole (HOBt), and suitable amine base, such as triethylamine and the like, in an inert solvent such as methylene chloride, chloroform, dimethylformamide, or mixture thereof, at or near room temperature for a period of 3 to 24 hours provided the corresponding coupled product 12. Following the same route described above for scheme e, the aryl group can then be installed.

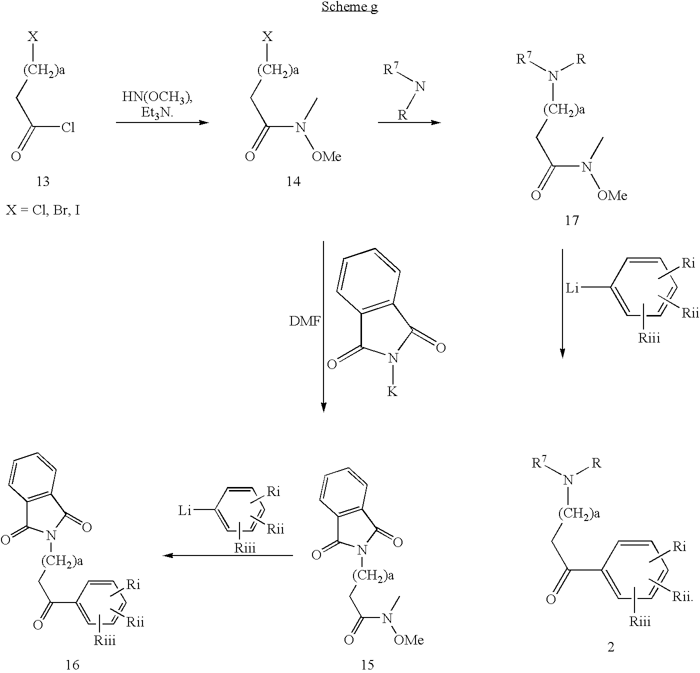

Scheme g illustrates another method for the synthesis of ketone such as 2 and 16, where the nitrogen group is introduced at a latter stage. As above a Weinreb amide 14 can be synthesised from an acid chloride. Treatment with the required amine, in an inert solvent such as THF, toluene, water and the such like can displace the group X to give 17. As above the aryl group can be introduced by displacement of the Weinreb amide with a suitable aryl lithium nucleophile. Alternatively the nitrogen atom can be introduced already protected as a phthalimide by displacement of the group X by potassium phthalimide, or similar salt thereof, by heating in an inert polar solvent such as DMF, DMSO, THF, toluene with or without the presence of a catalyst such as tetrabutylammonium iodide and the such like, to yield the compound 15. Again displacement of the Weinreb amide with an organolithium species completes the synthesis of ketone 16 suitable for cyclization under the Fischer condition described above for thienopyrrole synthesis.

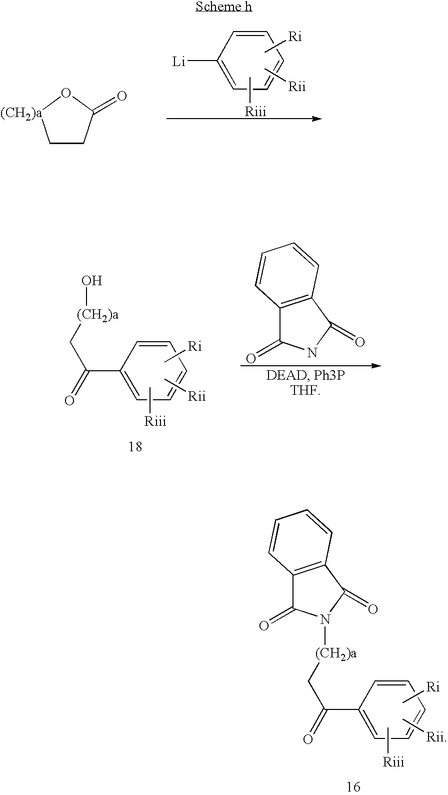

An alternative approach to a phthalimide protected nitrogen ketone, such as 16, can be taken by firstly treating a lactone, with an organolithium species as in the above schemes in a suitable solvent such as THF or ether at a low temperature of between −100° C. and −50° C. to yield a primary alcohol 18 (Scheme h). The hydroxyl function of 18 is replaced with a phthalimide group by a Mitsunobu reaction with an activating agent such as diethyldiazocarboxylate (DEAD), diisopropyldiazocarboxylate or the like with triphenylphosphine, tri-butylphosphine and the like, in an inert solvent such as benzene, toluene, tetrahydrofuran or mixtures thereof to give the desired ketone 16.

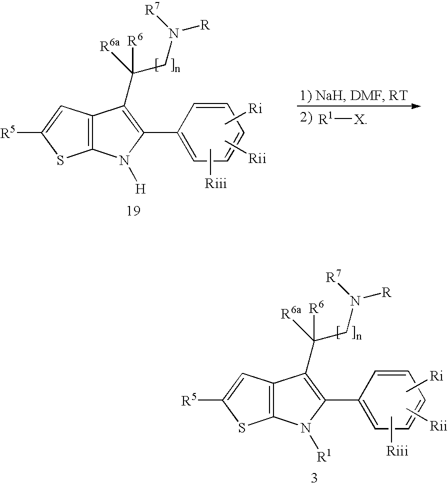

If the group R¹ was not present on the starting hydrazine before cyclization to form a thienopyrrole it may be added post cyclization by an alkylation reaction (19→3). The thienopyrrole is de-protonated by a strong base, such as sodium hydride, n-butyl lithium, lithium diisopropylamine, sodium hydroxide, potassium tert-butoxide in a suitable inert solvent such as THF, DMF, DMSO and the such like, and an alkyl halide added and the mixture stirred at room temperature.

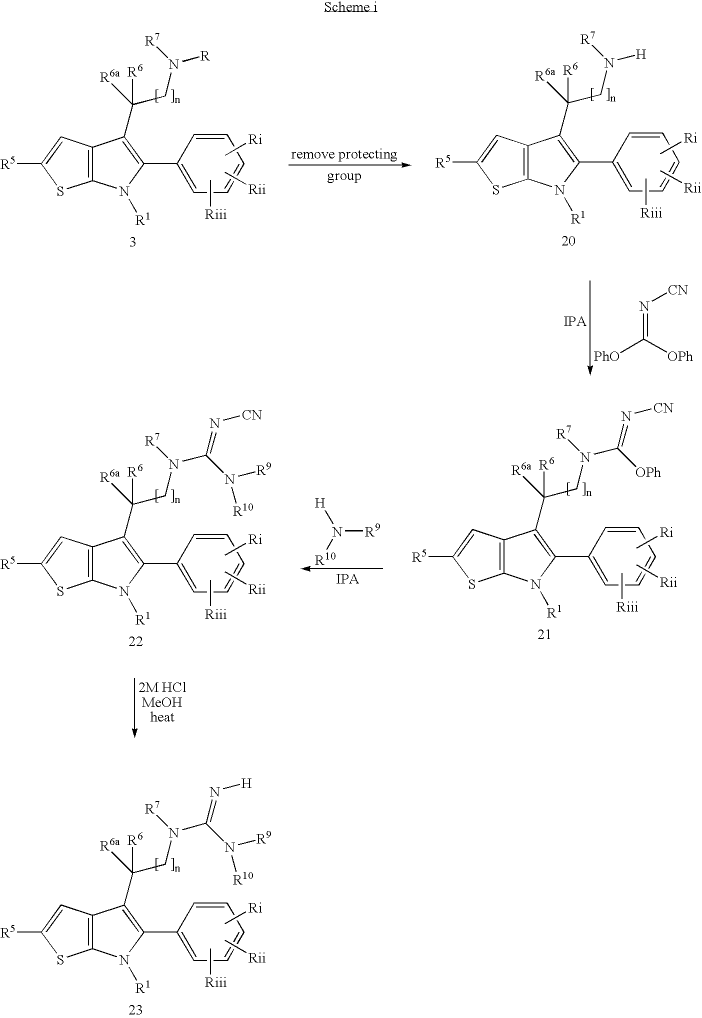

Depending on the route used above a thienopyrrole 20 suitable for conversion to a cyano-guanidine can be formed by removal of the protecting group, for example if a tert-butylcarbamate group was used then removal is accomplished using a strong acid, for example trifluoroacetic acid or hydrochloric acid in an inert solvent such as methylene chloride, chloroform, THF or dioxane at a temperature between −20° C. and 25° C. A phthalimide group, for example, can be removed by hydrazine in a suitable solvent for example methanol, ethanol, methylene chloride, chloroform, THF dioxane at a temperature between −20° C. and 25° C. The primary amine 20 can be converted to a cyano-guanidine 22 by the two step process of reaction with diphenyl cyanocarbonimidate in an inert organic solvent such as iso-propyl alcohol, methylene chloride, chloroform, benzene, tetrahydrofuran and the like, at a temperature between −20° C. and 50° C., followed by condensation with an appropriately substituted amine in an inert organic from the list above, with heating at a temperature between −20° C. and 100° C. (Scheme i 20→21→22). Further treatment of 22 with 2 molar Hydrochloric acid in methanol at elevated temperature yields guanidine compounds 23.

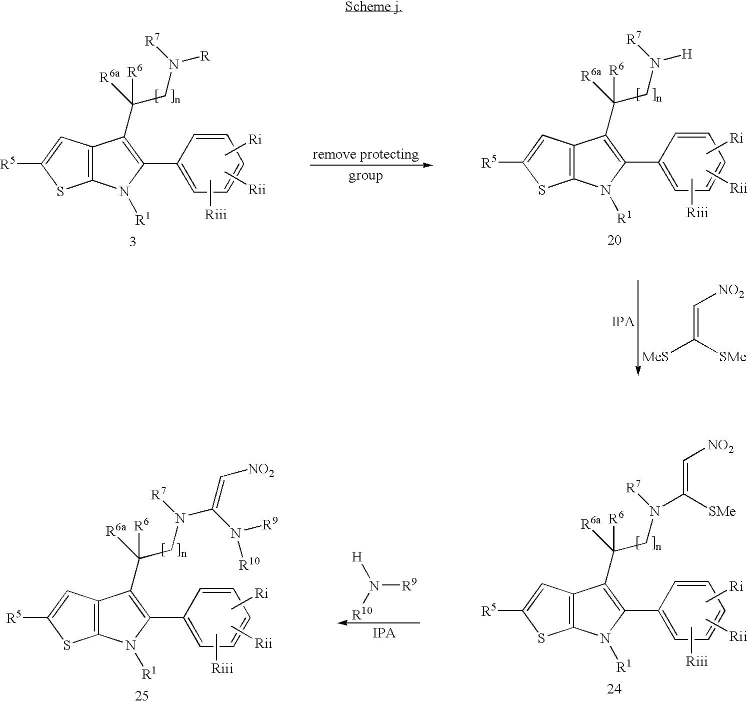

Similarly, reaction with 1,1′-bis(methylthio)-2-nitroethylene in an inert solvent such methylene chloride, chloroform, benzene, tetrahydrofuran and the like, followed by condensation with an appropriately substituted amine in an inert organic solvent from the list above yields the nitroethyleneimidazo[1,2-a]pyridine 25 (Scheme j, 20→24→25).

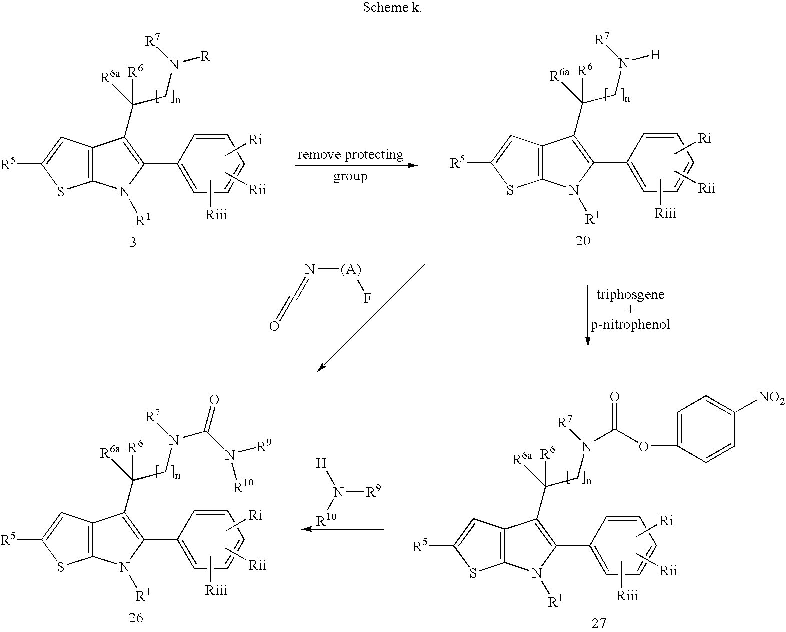

Again in a similar fashion the suitable thienopyrrole 20, derived from de-protection, can be converted to a urea by either direct treatment with an iso-cyanate in an inert solvent such as methylene chloride, chloroform or THF and the such like, or by a two step procedure of reaction with triphosgene (20→27) followed by addition of an amine (27→26), bearing the required substitution to yield 26.

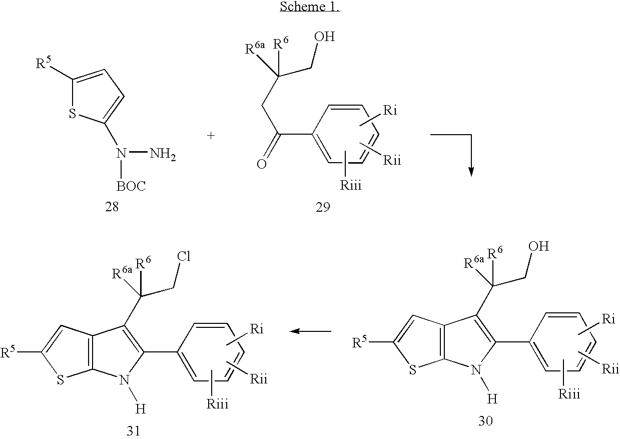

Chloro thieno-pyrrole intermediates, such as 31, can be made as shown in Scheme 1. 30 can synthesized by the classic Fisher thieno-pyrrole synthesis reaction by the condensation of a hydrazine-HCl 28 and a ketone 29, bearing hydrogen atoms α to the carbonyl. Treatment of these reactants in a suitable solvent, such as acetic acid, ethanol, sec-butanol, toluene, in the presence of an acid, such as sulphuric, hydrochloric, polyphosphoric and/or a Lewis acid, for example, boron trifluoride, zinc chloride, magnesium bromide, at elevated temperatures (for example 100° C.), gives the desired product. The chloro intermediate 31 can then be synthesized from 30 using, for example, either (i) sulphonyl chloride in methylene chloride at a temperature of about 0° C., or (ii) CCl₄ followed by triphenylphosphine in a solvent such as acetonitrile at a temperature of about 0° C. Thienopyrroles of the invention can then be prepared by displacement of chlorine atom using an appropriate side chain intermediate such as a substituted heterocyclic ring.

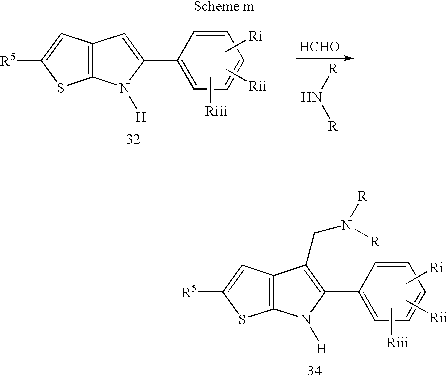

Thienopyrroles of Formula (I) wherein A is a direct bond and R⁶ and R^{6a} are both hydrogen can be prepared as shown in Scheme m. A thieno-pyrrole 32 can be reacted with formaldehyde and an amine, in a suitable solvent such as acetic acid/dioxan at a temperature of about 0° C. to 25° C. for between about 1 to 8 hours, to form the thieno-pyrrole 34.

Thienopyrroles such as 3, 7, 23, 25, 26 and 34 can then be used to prepare the corresponding pyrrole by reduction, for example, as shown in Scheme n for the conversion of the thienopyrrole 34 to the pyrrole 35. Reduction conditions such as with Raney-Nickel under hydrogen in a suitable solvent, such as ethanol or methanol, at a temperature between room temperature and the boiling point of the solvent can be used.

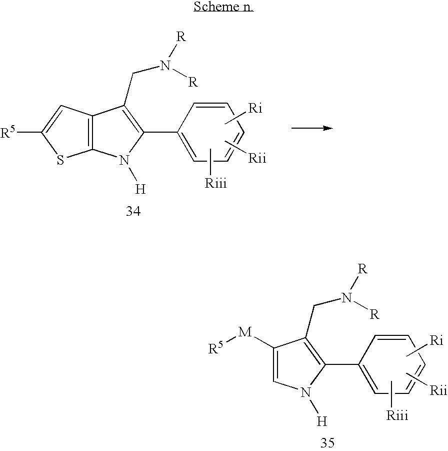

A pyrrole, such as 35, can be substituted at the 5-position with an R⁴ group using an appropriate electrophilic reagent reaction, as shown in Scheme o. For example, when R⁴ is a halogen, such as chlorine, an electrophilic reagent such as N-chlorosuccinimide in a suitable solvent, such as THF, at a temperature between room temperature and the boiling point of the solvent can be used, or when R⁴ is alkyl, such as ethyl, an electrophilic reagent such as an appropriate alkyl halide, such as ethyl iodide, can be used under Friedel Craft conditions, for example in the presence of diethylaluminum chloride in a suitable solvent, such as CH₂Cl₂, in an inert atmosphere such as nitrogen, at a temperature between room temperature and the boiling point of the solvent.

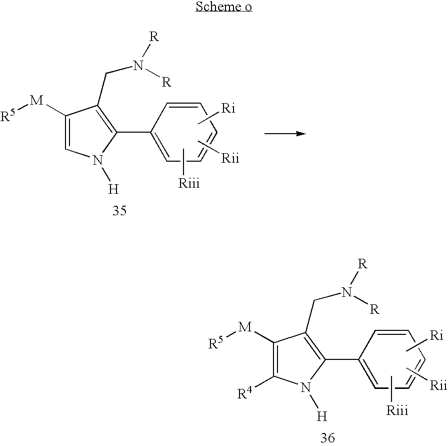

### EXAMPLES

The invention will now be illustrated with the following non-limiting Examples in which, unless otherwise stated:

(i) evaporations were carried out by rotary evaporation in vacuo and work-up procedures were carried out after removal of residual solids such as drying agents by filtration;

(ii) operations were carried out at room temperature, that is in the range 18-25° C. and under an atmosphere of an inert gas such as argon or nitrogen;

(iii) yields are given for illustration only and are not necessarily the maximum attainable;

(iv) the structures of the end-products of the Formula (I) were confirmed by nuclear (generally proton) magnetic resonance (NMR) and mass spectral techniques; proton magnetic resonance chemical shift values were measured on the delta scale and peak multiplicities are shown as follows: s, singlet; d, doublet; t, triplet; m, multiplet; br, broad; q, quartet, quin, quintet;

(v) intermediates were not generally fully characterised and purity was assessed by thin layer chromatography (TLC), high-performance liquid chromatography (HPLC), infra-red (IR) or NMR analysis;

(vi) chromatography was performed on silica (Merck Keiselgel: Art.9385);

(vii) isolute™ refers to silica (SiO₂) based columns with irregular particles with an average size of 50 μm with nominal 60 Å porosity [Source: Jones Chromatography, Ltd., Glamorgan, Wales, United Kingdom].

Abbreviations

Atm Atmospheres
boc t-butoxycarbonyl
DCM dichloromethane
DEAD diethylazodicarboxylate
DIPEA diisopropylethylamine
DMA dimethylacetamide
DMAP 4-dimethylaminopyridine
DMSO dimethyl sulphoxide
DMF dimethylformamide
DTAD Di-tert-butyl azodicarboxylate
EDC 1-(3-dimethylaminopropyl)-3-ethylcarbodiimide hydrochloride
HATU O-(7-azabenzotriazol-1-yl)-N,N,N′,N′-tetramethyluronium hexafluorophosphate
THF tetrahydrofuran

### Example 1

### 3-[3,3-Dimethyl-4-oxo-4-(azabicyclo[2.2.1]heptan-7-yl)butyl]-4-[2-{4-(morpholin-4-ylcarbonyl)piperidin-1-yl}ethyl]-5-(3,5-dimethylphenyl)-1H-pyrrole

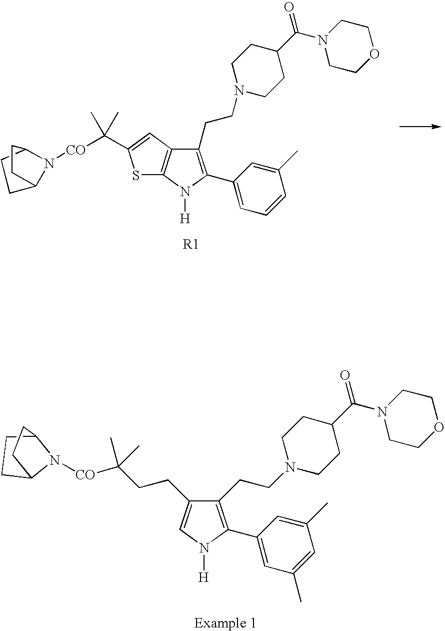

A suspension of R1 (100 mg; 0.162 mmol) in EtOH (50 ml) was treated with Raney-Nickel (5 g) and placed under an atmosphere of hydrogen (1.7 atm.). The mixture was stirred at room temperature for 16 hours. The mixture was filtered, the filtrate concentrated and the residue purified by flash chromatography eluting with ammonia in MeOH(7N)/CH₂Cl₂ (1/10) to give Example 1 as a white foam (50 mg).

Yield: 52%

¹H NMR spectrum (CDCl3): 1.29 (s, 6H); 1.47 (m, 4H); 1.67 (m, 2H); 1.79 (m, 4H); 1.88 (m, 4H); 2.07 (m, 2H); 2.33 (s, 6H); 2.44 (m, 3H); 2.5 (m, 2H); 2.77 (m, 2H); 3.04 (m, 2H); 3.48 (m, 2H); 3.62 (m, 2H); 3.67 (s br, 4H); 4.66 (s br, 2H); 6.56 (d, 1H); 6.90 (s, 1H); 7.03 (s, 2H); 7.92 (s br, 1H).

MS-ESI: 589 [M+H]⁺

The starting material was prepared as follows:—

### 2-[1,1-dimethyl-2-oxo-2-azabicyclo[2.2.1]heptan-7-ylethyl]-4-[2-(4-{morpholinocarbonyl}piperidin-1-yl)ethyl]-5-(3,5-dimethylphenyl-6H -thieno[2,3-b]pyrrole (R1)

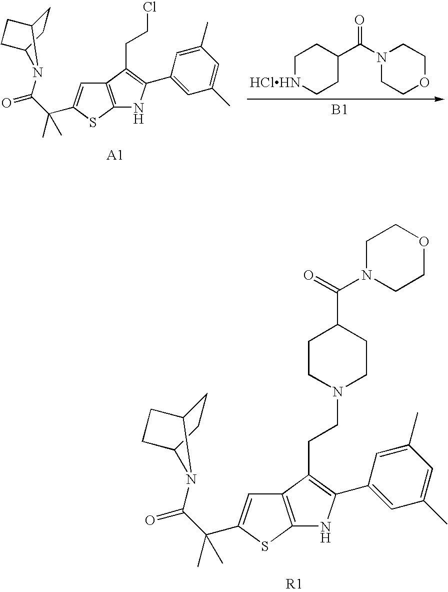

A mixture of A1 (0.137 g; 0.3 mmol), B1 (0.150 g, 0.6 mmol), K₂CO₃ (0.125 g; 0.9 mmol) and NaI (0.045 g; 0.3 mmol) in dimethylacetamide (3 ml) was heated at 85° C. under an argon atmosphere for 6 hours. The crude mixture was purified on preparative LC-MS (column Symmetry C₁₈, AcOH buffer, H₂O—CH₃CN gradient) and the residue was evaporated and crystallised in a pentane-Et₂O mixture to give R1 as a white solid.

Yield: 55%

¹H NMR (CDCl₃): 1.28 (m, 4H); 1.62 (s, 6H); 1.50-1.75 (m, 6H); 1.93 (m, 2H); 2.08 (m, 2H); 2.35 (s, 6H); 2.48 (m, 1H); 2.68 (m, 2H); 2.97 (br s, 2H); 3.09 (m, 2H); 3.50 (s, 2H); 3.62 (s, 2H); 3.68 (s, 4H); 4.12 (br s, 1H); 4.75 (br s, 1H); 6.75 (s, 1H); 6.94 (s, 1H); 7.06 (s, 2H); 8.13 (s, 1H).

MS-ESI: 617 [M+H]⁺
The intermediate A1 was prepared as follows:— 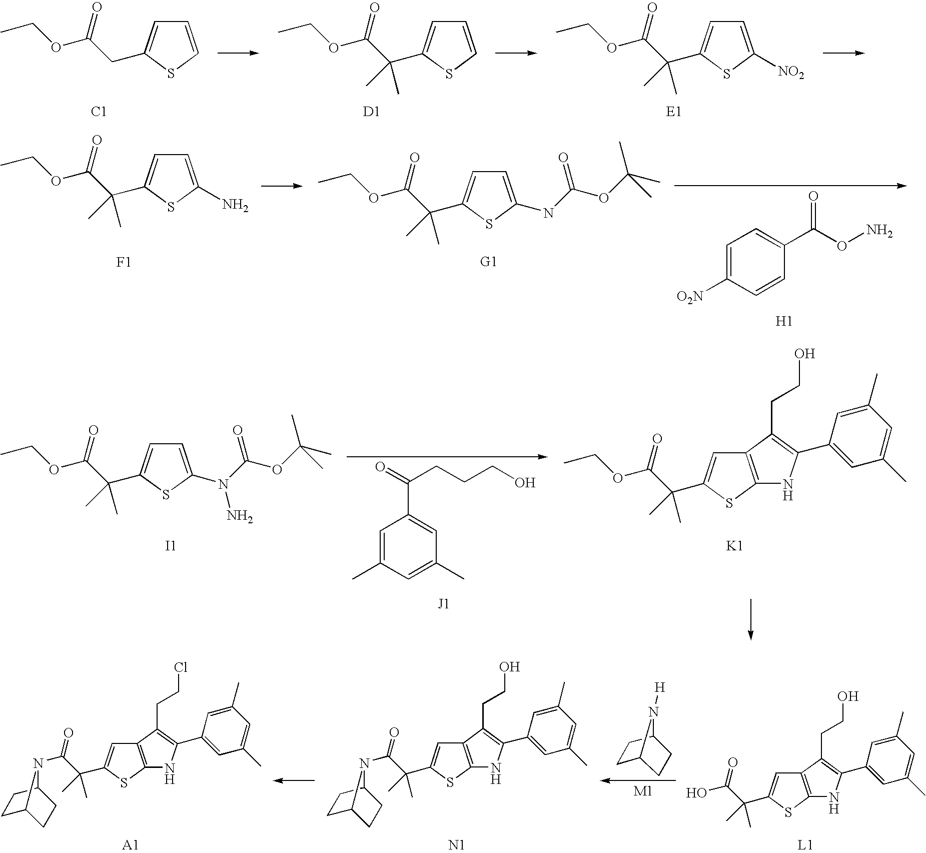
To a suspension of NaH (54 g; 1.35 mol) and 18-crown-6 in THF (2 l) stirred at ambient temperature under an argon atmosphere, was added C1 (100 g; 0.588 mol) over a period of 30 minutes. After stirring overnight, the mixture was cooled to 0° C. and methyl iodide was added dropwise. The mixture was stirred at 18° C. for 3 hours, poured into a saturated solution of NH₄Cl and extracted with AcOEt. The organic phase was evaporated and purified by flash chromatography eluting with petroleum ether/ethyl acetate 95/5 to give D1 as an oil.

Yield: 90%

¹H NMR(CDCl₃): 1.20 (t, 3H); 1.63 (s, 6H); 4.10 (q, 2H); 6.92 (m, 2H); 7.17 (m, 1H).

Nitronium tetrafluoroborate (77.9 g; 0.586 mol) was added at −55° C. to a solution of D1 (105.6 g; 0.583 mol) in DME (1.5 l). The mixture was allowed to warm up to −10° C. over 4 hours. After extraction with ethyl acetate, the organic phase was purified by flash chromatography, eluting with petroleum ether/AcOEt 95/5 to give E1.

Yield: 86%

¹H NMR (CDCl₃): 1.23 (t, 3H, 1.65 (s, 6H); 4.14 (q, 2H); 6.90 (d, 1H); 7.75 (d, 1H).

A suspension of E1 (101.7 g; 0.41 mol) and 10% Pd/C (15 g) in a mixture of ethanol (700 ml) and ethyl acetate (300 ml) was hydrogenated under a hydrogen atmosphere (1.5 atm.) for 5 hours. After filtration of the catalyst through celite, the residue was evaporated and redissolved in THF (900 ml). Di-tert-butyl dicarbonate (100 g; 0.46 mol) was added and the mixture was refluxed for 16 hours. After evaporation of the solvents, the resulting solid was triturated with petroleum ether and filtered to give G1.

Yield: 68%

¹H NMR (CDCl₃): 1.20 (t, 3H); 1.48 (s, 9H); 1.58 (s, 6H); 4.10 (q, 2H); 6.30 (m, 1H); 6.60 (m, 1H).

To a suspension of sodium hydride (44.6 g; 1.12 mol) in DMF (700 ml) at 10° C., was added a solution of G1 (290 g; 930 mmol) in DMF (1 l) over a period of 5 minutes. The resulting orange suspension was allowed to warm to room temperature and stirred for 2 hours. The resulting solution was cooled to −5° C. in an acetone/ice bath and a solution of H1 (201 g; 1.02 mol) in DMF (1.4 1) was added over a period of 1 hours. During this period additional DMF (1 l) was added to mobilise the thick precipitate which formed. The resulting suspension was allowed to warm to room temperature and stirred overnight after which HPLC showed no remaining starting material. The suspension was poured into water (6 litres) and extracted with diethyl ether (3×2 l). The organic extracts were combined and concentrated to approximately 3 litres and washed with water (4×1.5 l), a saturated solution of brine (1 l), dried over magnesium sulfate and evaporated to dryness to afford the free base as an off-white solid in quantitative yield. To a stirred solution of the free base (150 g; 457 mmol) in diethyl ether (1.2 l) and heptane (600 ml) at 0° C., was added a 4.0M solution of HCl in 1,4-dioxane (145 ml; 570 mmol) over a period of 1 hours. The resulting thick, white precipitate was collected by filtration, washed with a mixture of diethyl ether-heptane (1:1, 500 ml) and dried to a constant weight to afford the I1.HCl (160.3 g) as a white solid.

Yield: 96%

MS-ESI: 328 [M+H]⁺

To a stirred solution of I1 (141 g; 380 mmol) in 2-butanol (1.3 l) was added J1 (104 g; 540 mmol) and zinc chloride (106 g; 770 mmol). The resulting suspension was heated at 100° C. for 8 hours after which HPLC showed no remaining starting material. The resulting dark brown solution was evaporated to dryness. The resulting dark brown residue was dissolved in CH₂Cl₂ (100 ml), filtered and the filtrate was purified by flash chromatography eluting with CH₂Cl₂, ethyl acetate (9/1) to afford K1 (98 g) as a brown solid.

Yield: 67%

MS-ESI: 386 [M+H]⁺

To a stirred solution of K1 (98 g; 254 mmol) in ethanol (1.8 l) was added 1N NaOH (1.27 l; 1.270 mol). The resulting solution was heated at 60° C. for 4 hours after which HPLC showed no remaining starting material. The reaction mixture was cooled to room temperature and the ethanol was evaporated. The resulting brown solution was cooled to 5° C. and concentrated HCl was added dropwise with rapid agitation decreasing the pH to 1. The resulting precipitate was collected by filtration, washed to a neutral pH with water (3×1 l) and dried to a constant weight in a vacuum oven at 50° C. to afford L1 as a beige solid (68.3 g).

Yield: 75%

MS-ESI: 358 [M+H]⁺

To a stirred solution of L1 (35.7 g; 100 mmol) and M1 (57 g; 150 mmol) in CH₂Cl₂ (1 l) at 0° C., was added DIPEA (70 ml; 400 mmol) and solid HATU (57 g; 150 mmol) over a period of 15 minutes. The reaction mixture was allowed to warm to room temperature and stirred for 2 hours after which HPLC showed no remaining starting material. The reaction mixture was washed with a saturated aqueous solution of citric acid (350 ml), a saturated solution of sodium bicarbonate (350 ml) and water (3×350 ml). The organic layer was dried over anhydrous magnesium sulfate, filtered, and evaporated to dryness. The resulting oily residue was triturated with ethyl acetate (100 ml) and the resulting precipitate collected by filtration and dried to a constant weight in a vacuum oven at 40° C. to afford N1 (31.4 g) as a beige solid.

Yield: 69%

MS-ESI: 437 [M+H]⁺

To a stirred solution of N1 (29.7 g; 68.1 mmol) in CH₂Cl₂ (700 ml) at 0° C. was added dropwise neat thionyl chloride (6 ml; 81.7 mmol). The mixture was allowed to warm to room temperature and stirred for a period of 2 hours after which HPLC showed no remaining starting material. The reaction mixture was evaporated and purified by flash chromatography, eluting with CH₂Cl₂/AcOEt (9/1) to give A1 as beige foam. The foam was triturated with diethyl ether (100 ml) and the resulting solid collected by filtration, washed with diethyl ether (2×50 ml) and dried to a constant weight in a vacuum oven at 40° C. to afford A1 as a white solid (26.5 g).

Yield: 85%

MS-ESI: 454 [M+H]⁺

¹H NMR (DMSO-d₆) 1.19-1.41 (m, 4H); 1.45-1.59 (m, 10H); 2.32 (s, 6H); 3.14 (t, 2H); 3.83 (t, 4H); 4.13 (br s, 1H); 4.43 (br s, 1H); 6.89-6.93 (two overlapping s, 2H); 7.08 (s, 2H).

The intermediate amine M1 was synthesised as follows:—

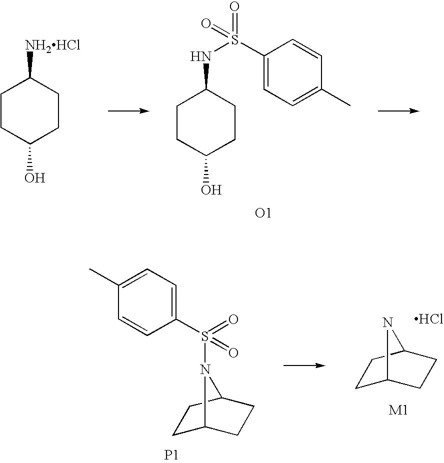

To a stirred suspension of trans-4-aminocyclohexanol (300 g; 1.98 mol) in isopropanol (3.5 l) at 0° C. was added triethylamine (1.1 l; 7.92 mol) followed by solid p-toluenesulfonyl chloride (377 g; 1.98 mmol) over a period of 30 minutes. The reaction mixture was heated at 60° C. for 2 hours after which HPLC showed no remaining starting material. The resulting suspension was cooled to room temperature and the precipitate of triethylamine hydrochloride removed by filtration. The filtrate was evaporated to dryness to afford a colourless oil which was dissolved in ethyl acetate (3 l), washed with 0.5N HCl (800 ml), water (1.5 l) and dried over MgSO₄. The solvent was evaporated on a rotary evaporator to afford O1 (456.5 g) as a white crystalline solid.

Yield: 86%

MS-ESI: 270 [M+H]⁺

To a stirred solution of O1 (600 g; 2.23 mol) in THF (2 l) at −10° C. in an ice/acetone bath, was added triphenylphosphine (700 g; 2.67 mol) followed by di-tert-butyl azodicarboxylate (DTAD) (564 g; 2.45 mol) in THF (1.5 l) over a period of 1.5 hours maintaining the internal temperature below 10° C. The ice/acetone bath was removed and reaction mixture was allowed to warm to room temperature over a period of 1.5 hours after which HPLC showed no remaining starting material. The reaction mixture was evaporated to dryness and the residue was crystallised from hot MeOH (2.8 l). The resulting crystalline suspension was cooled to 0° C. and the crystals collected by filtration, washed with cold MeOH (2×200 ml) and dried to a constant weight in a vacuum oven to afford P1 (378.2 g) as a white crystalline solid routinely contaminated with approximately 10% (w/w) of triphenylphosphine oxide.

Yield: 68%

MS-ESI: 252 [M+H]⁺

In two separate batches:—To a stirred solution of P1 (380 g; 1.51 mol) in THF (3 l) at 0° C. was added solid pellets of lithium aluminium hydride (229.4 g; 6.04 mol) over a period of 2 hours under nitrogen. The resulting grey suspension was allowed to warm to room temperature and stirred for 4 days after which HPLC showed no remaining starting material. The reaction mixture was diluted with THF (1 l), cooled to 0° C. and solid sodium sulfate decahydrate was added over a period of 2 hours with rapid agitation. When the effervescence had subsided, the resulting suspension was filtered and the filtrate acidified with gaseous HCl affording a thick white precipitate which was collected by filtration, washed with THF (2×500 ml) and dried to a constant weight to afford M1 (batch 1: 86.8 g; 43%) (batch 2: 97.3 g; 49%) as a white solid. The filter cakes obtained from the first filtration were suspended in 6N NaOH (400 ml) and filtered. The filtrate was extracted with diethyl ether (4 1). The organic layer was acidified with gaseous HCl affording a thick white precipitate which was collected by filtration, washed with diethyl ether (2×500 ml) and dried to a constant weight in a vacuum oven at 40° C. to afford M1.HCl (105.9 g) as a white solid.

Yield: 72%

¹H NMR (DMSO d₆) 1.57 (m, 4H); 1.86 (m, 4H); 4.12 (s, 2H); 8.8-9.05 (s br, 1H).

### Example 2

### 3-[3,3-Dimethyl-4-oxo-4-(azabicyclo[2.2.1]heptan-7-yl)but-2-en-1-yl]-4-[1S-methyl-2-(N′-isopropoxycarbonyl-3-pyrid-4-yl-pyrrolidin-1-ylcarboximidamido)ethyl]-5-(3,5-dimethylphenyl)-1H-pyrrole

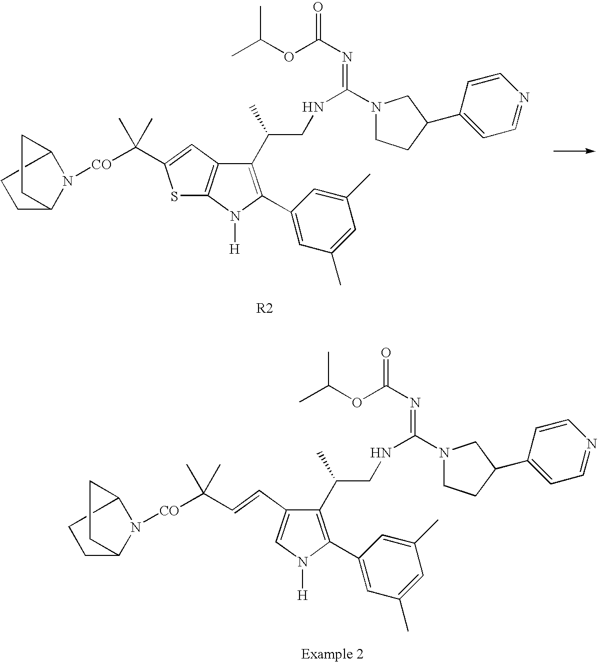

Example 2 was synthesised by the method used for preparing Example 1, except that MeOH was used as the solvent. The following quantities of starting material and conditions were used: R2 (600 mg; 0.85 mmol); MeOH (30 ml); RaNi (12 g); hydrogen (1.7 atm.); 16 hours. Example 2 was obtained as a yellow foam (120 mg).

Chromatography: Ammonia in MeOH(7N)/CH₂Cl₂ (1/20)

Yield: 21%

¹H NMR spectrum (CDCl₃): 1.2-1.26 (m, 9H); 1.35-1.4 (m, 12H); 1.75 (m, 4H); 2.2 (m, 1H); 2.3 (s, 6H); 3.05-3.5 (m, 7H); 3.65 (m, 1H); 4.63 (s br, 2H); 4.84 (m, 1H); 6.03 (dd, 1H); 6.45 (dd, 1H); 6.83 (d, 1H); 6.93 (m, 3H); 7.06 (dd, 2H); 8.01 (s, 1H); 8.52 (m, 2H).

MS-ESI: 679 [M+H]⁺

The starting material was prepared as follows:—

### 2-(1,1-dimethyl-2-oxo-2-azabicyclo[2.2.1]heptan-7-ylethyl)-4-[1S-methyl-2-(N′-isopropoxycarbonyl-3-pyrid-4-yl-pyrrolidin-1-ylcarboximidamido)ethyl]-5-(3,5-dimethylphenyl)-6H-thieno[2,3-b]pyrrole (R2)

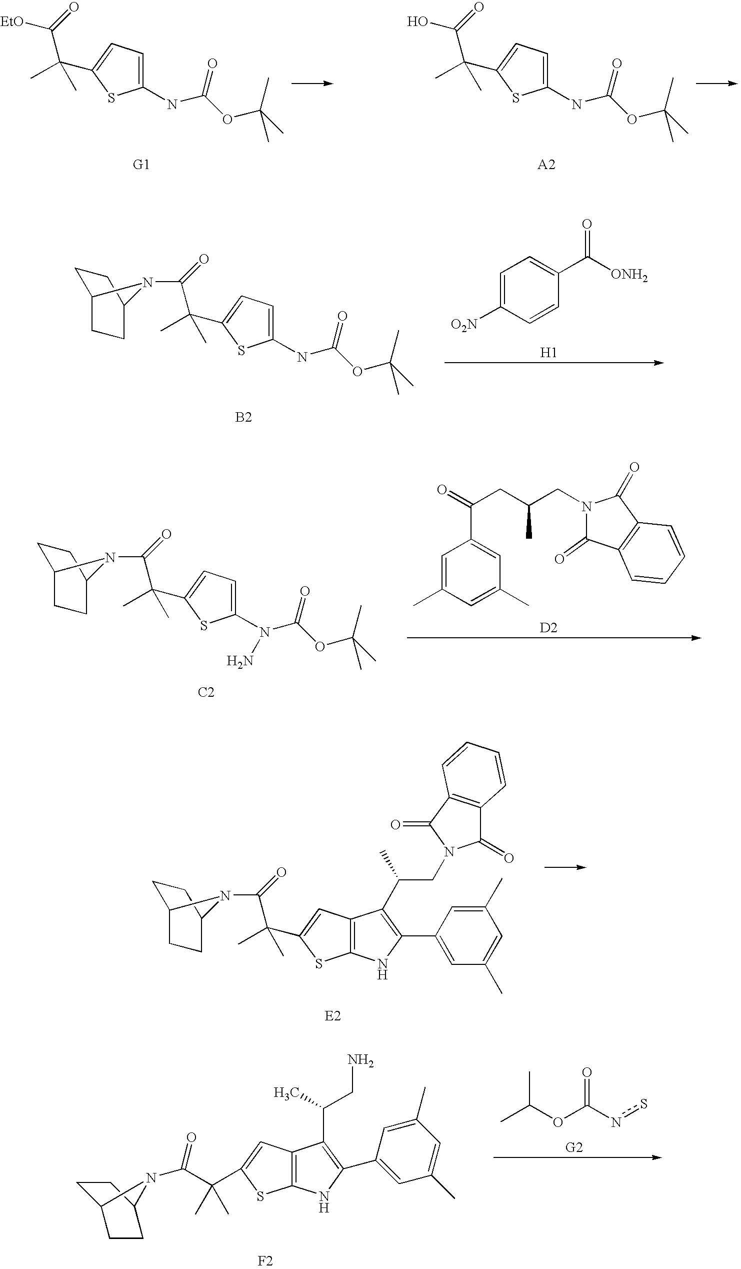

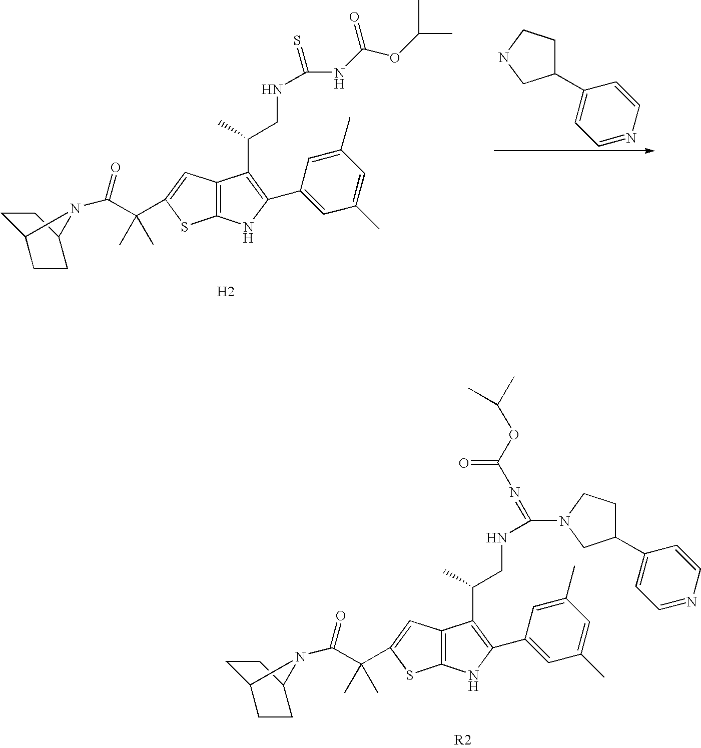

A solution of G1 (50 g; 0.16 mol) and 2N NaOH (160 ml) in EtOH (300 ml) was refluxed for 1 hours 30 minutes. After evaporation to dryness, the residue was partitioned between water and ether. The aqueous layer was acidified with saturated citric acid and extracted with EtOAc to give after evaporation a solid, which was triturated in pentane and filtered to give A2 as a solid.

Yield: 100%

¹H NMR (DMSOd₆): 1.48 (m, 15H); 6.30 (d, 1H); 6.59 (d, 1H).

A solution of A2 (172 g; 0.6 mol), EDCl (172 g; 0.9 mol) and DMAP (22 g; 0.18 mol) in CH₂C₂ (1.5 l) was stirred under an argon atmosphere for 10 minutes. Solid M1.HCl (88 g; 0.66 mol) was then added and the mixture was stirred overnight at ambient temperature. After evaporation to dryness, the residue was partitioned between EtOAc and water. The organic phase was evaporated and the solid residue was triturated with diethyl ether, filtered, washed with diethyl ether and dried to a constant weight in a vacuum oven to afford B2 (153.7 g) as a beige solid.

Yield: 70.5%

¹H NMR (DMSOd₆): 1.28 (m, 4H); 1.44 (m, 19H); 4.0 (s br, 1H); 4.4 (s br, 1H); 6.33 (d, 1H); 6.53 (d, 1H); 10.29 (s, 1H).

A suspension of B2 (153.7 g; 0.42 mol) in DMF (1.2 l) was added under an argon atmosphere to a suspension of NaH 60% (20.2 g; 0.5 mol) in DMF (400 ml). The mixture was stirred 1.5 hours, cooled to 5° C. and a solution of H1 (92.1 g; 0.5 mol) in 1,4-dioxane (4 l) was added. The reaction mixture was stirred at ambient temperature overnight. After filtration of the insoluble, the filtrate was evaporated to dryness and the residue was partitioned between Et₂O and water. The organic phase was mostly evaporated and the solid precipitate was filtered, washed with a saturated solution of NaHCO₃, pentane and dried to a constant weight in a vacuum oven to give C2 (127.2 g) as a white solid.

Yield: 80%

¹H NMR (CDCl₃): 1.31 (m, 4H); 1.56 (s, 15H); 1.6 (m, 4H); 4.0 (s br, 1H); 4.51 (s, 2H); 4.7 (d, 2H); 6.54 (d, 1H); 6.70 (s br, 1H).

To a stirred suspension of C2 (17 g; 45 mmol) and D2 (22.5 g; 67.0 mmol) in 2-butanol (50 ml) was added a 4M solution of HCl in 1,4-dioxane (22.5 ml; 90.0 mmol). The resulting thick suspension was heated at 90° C. for 1 hour after which HPLC showed no remaining starting material. The resulting dark brown solution was evaporated to dryness on a rotary evaporator and the residue was dissolved in CH₂Cl₂ and purified by flash chromatography on silica gel eluting with EtOAc/hexanes (10-50% EtOAc) to afford E2 (16 g) as a white solid.

Yield: 54%

¹H NMR (CDCl₃), 1.26-1.40 (m, 7H); 1.57 (s, 6H); 1.63-1.83 (m, 4H); 2.32 (s, 6H); 3.66 (m, 1H); 3.87 (m, 2H); 4.20 (s br, 1H); 4.75 (s br, 1H); 6.86 (s, 1H); 6.92 (s, 1H); 6.98 (s, 2H); 7.67 (m, 2H); 7.74 (m, 2H); 8.03 (s, 1H).

MS-ESI: 580 [M+H]⁺

To a stirred suspension of E2 (14 g; 24 mmol) in EtOH (300 ml) at room temperature, was added neat hydrazine monohydrate (12 ml; 240 mmol). The reaction mixture was stirred at room temperature for 16 hours after which HPLC showed no remaining starting material. The resulting precipitate was collected by filtration, washed with EtOH (2×20 ml) and the filtrate was evaporated to dryness on a rotary evaporator and dried to a constant weight under high vacuum to afford F2 (10.9 g) as a yellow foam which was used without further purification.

Yield: 100%

¹H NMR (CDCl₃): 1.27-1.36 (m, 7H); 1.43-1.53 (m, 10H); 2.34 (s, 6H); 3.06 (m, 1H) 3.23 (m, 1H); 3.28 (m, 1H); 4.10 (s br, 1H); 4.50 (s br, 1H); 6.87 (s, 1H); 6.96 (s, 1H); 7.07 (s, 2H).

MS-ESI: 450 [M+H]⁺

To a solution of G2 (0.098 g; 0.66 mmol) in CH₂Cl₂ (1.5 ml) at 0° C. was added a solution of F2 (300 mg; 0.66 mmol) in CH₂Cl₂ (1.5 ml). The mixture was stirred at ambient temperature for 90 minutes and extracted. The organic layer was evaporated and purified by flash chromatography, eluting with CH₂Cl₂/AcOEt (50/50) to give H2.

Yield: 90%

¹H NMR (CDCl₃): 1.2 (m, 6H); 1.2-1.35 (m, 4H); 1.38 (d, 3H); 1.50-1.80 (m, 4H); 1.55 (s, 6H); 2.35 (s, 6H); 3.45 (m, 1H); 1.78 (m, 1H); 4.00 (m, 1H); 4.10 (s br 1H); 4.75 (s br, 1H); 4.87 (m, 1H); 6.80 (s, 1H); 6.96 (s, 1H); 7.07 (s, 2H); 7.72 (s, 1H); 8.10 (s, 1H); 9.54 (s, 1H).

MS-ESI: [M+H]⁺ 595

To a solution of H2 (325 mg; 0.54 mmol) in CH₂Cl₂ (10 ml) at 0° C. was added under an argon atmosphere 4-pyrrolidin-3-yl pyridine (122 mg; 0.82 mmol), EDC (158 mg; 0.82 mmol) and DIPEA (0.142 ml; 0.82 mmol). The mixture was stirred at 0° C. for 15 minutes allowed to warm up and stirred for 24 hours at ambient temperature. The reaction mixture was extracted with CH₂Cl₂. The organic layer was evaporated and the crude material was purified by flash chromatography eluting with ammonia in MeOH(7N)/CH₂Cl₂ (1/20) to give R2 as a solid.

Yield: 55%.

¹H NMR (CDCl₃): 1.2 (m, 6H); 1.2-1.3 (m, 4H); 1.4 (m, 3H); 1.5-1.68 (m, 4H); 1.55 (s, 6H); 1.7-1.9 (m, 1H); 2.1-2.2 (m, 1H); 2.28 (m, 6H); 3.02-3.5 (m, 7H); 3.6 (m, 1H); 4.05 (s br, 1H); 4.7 (s br, 1H); 4.82 (m, 1H); 6.72 (s, 1H); 6.9 (m, 1H); 7.0 (s, 1H); 7.05 (d, 2H); 8.20 (s, 1H); 8.50 (d, 2H).

MS-ESI: [M+H]⁺ 709

### Example 3

### 3-[3,3-Dimethyl-4-oxo-4-(azabicyclo[2.2.1]heptan-7-yl)butyl]-4-[1S-methyl-2-(N′-isopropoxycarbonyl-3-pyrid-4-yl-pyrrolidin-1-ylcarboximidamido) ethyl]-5-(3,5-dimethylphenyl)-1H-pyrrole

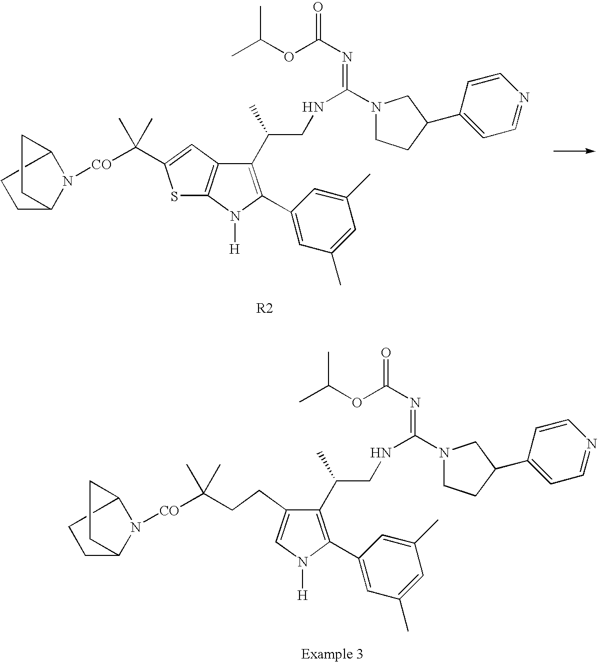

Example 3 was synthesised by the method used for preparing Example 1. The following quantities of starting material and conditions were used: R2 (300 mg; 0.425 mmol); EtOH (50 ml); RaNi (10 g); hydrogen (1.5 atm.); 3 days. Example 3 was obtained as a yellow foam (121 mg).

Chromatography: Ammonia in MeOH(7N)/CH₂Cl₂ (1/10)

Yield: 42%

¹H NMR spectrum (CDCl₃): 1.12-1.26 (m, 9H); 1.29 (s, 6H); 1.35 (t, 2H); 1.46 (m, 4H); 1.77 (m, 4H); 1.89 (m, 2H); 2.2 (m, 1H); 2.3 (s, 6H); 2.5 (m, 2H); 3.05-3.45 (m, 7H); 3.65 (m, 1H); 4.65 (s br, 2H); 4.84 (m, 1H); 6.57 (s, 1H); 6.92 (m, 3H); 7.08 (dd, 2H); 7.87 (s, 1H); 8.52 (m, 2H).

MS-ESI: 681 [M+H]⁺

### Example 4

### 3-[3,3-Dimethyl-4-oxo-4-(azabicyclo[2.2.1]heptan-7-yl)butyl]-4-[2-{4-(pyrrolidin-1-ylcarbonyl)piperazin-1-yl}ethyl]-5-(3,5-dimethylphenyl)-1H-pyrrole

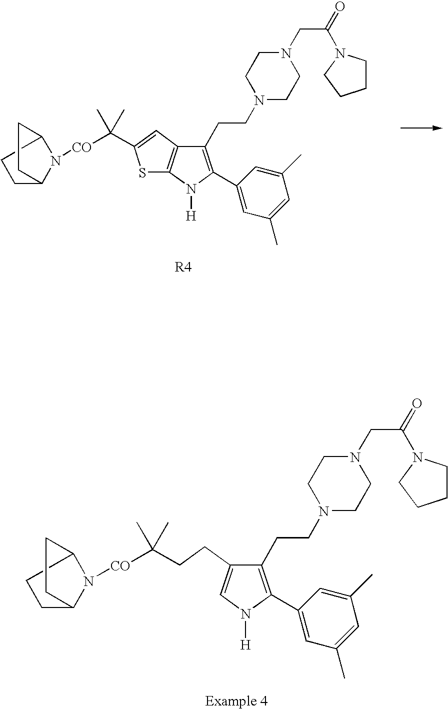

Example 4 was synthesised by the method used for preparing Example 1, except that MeOH was used as solvent and the reaction was carried out with no hydrogen atmosphere. The following quantities of starting material and conditions were used: R4 (300 mg; 0.488 mmol); MeOH (20 ml); RaNi (0.5 g); 3 days. Example 4 was obtained as a pale yellow foam (102 mg).

Chromatography: Ammonia in MeOH(7N)/CH₂Cl₂ (1/20)

Yield: 35%

¹H NMR spectrum (CDCl₃): 1.29 (s, 6H); 1.47 (m, 4H); 1.79 (m, 4H); 1.87 (m, 4H); 1.94 (t, 2H); 2.32 (s, 6H); 2.44 (m, 2H); 2.51 (m, 2H); 2.59 (m br, 8H); 2.77 (m, 2H); 3.11 (s, 2H); 3.48 (m, 4H); 4.66 (s br, 2H); 6.55 (d, 1H); 6.89 (s, 1H); 7.02 (s, 2H); 7.92 (s br, 1H).

MS-ESI: 588 [M+H]⁺

The starting material was prepared as follows:—

### 2-[1,1-Dimethyl-2-oxo-2-azabicyclo[2.2.1]heptan-7-ylethyl]-4-[2-{4-(pyrrolidin-1-ylcarbonylmethyl)piperazin-1-yl}ethyl]-5-(3,5-dimethylphenyl)-6H -thieno[2,3-b]pyrrole

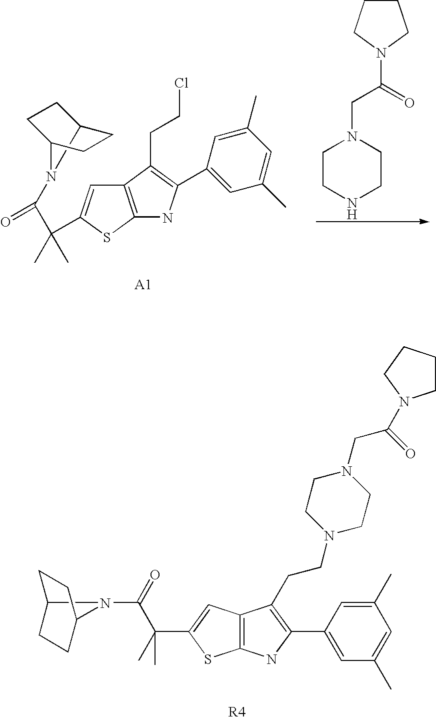

A mixture of A1 (see Example 1) (182 mg; 0.4 mmol), N-(pyrrolidinocarbonylmethyl)-piperazine (95 mg, 0.48 mmol), NaI (72 mg; 0.48 mmol) and K₂CO₃ (67 mg; 0.48 mmol) in acetonitrile (4 ml) was heated at 80° C. under argon atmosphere for 8 hours. The crude mixture was evaporated and purified by flash chromatography eluting with ammonia in MeOH(7N)/CH₂Cl₂ (1/20) to give after trituration in ether/pentane R4 as a solid.

Yield: 42%

¹H NMR (CDCl₃): 1.15-1.4 (m, 6H); 1.45-1.75 (m, 6H); 1.59 (s, 6H); 1.8-2 (m, 4H); 2.32 (s, 6H); 2.45-2.75 (m, 6H); 2.9-3 (m, 2H); 3.1 (s, 2H); 3.44-3.5 (m, 4H); 4-4.2 (m, br, 1H); 4.6-4.8 (m, br, 1H); 6.72 (s, 1H); 6.92 (s, 1H); 7.04 (s, 2H); 8.13 (s, 1H).

MS-ESI: 616 [M+H]⁺

### Example 5

### 2-Chloro-3-[3,3-Dimethyl-4-oxo-4-(azabicyclo[2.2.1]heptan-7-yl)butyl]-4-[2-{4-(pyrrolidin-1-ylcarbonyl)piperazin-1-yl}ethyl]-5-(3,5-dimethylphenyl)-1H-pyrrole

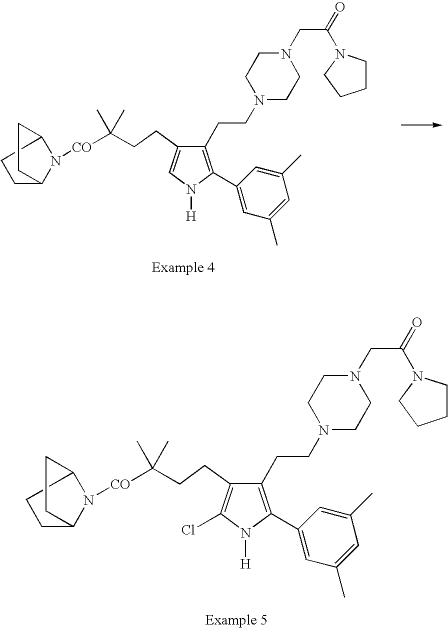

A solution of Example 4 (100 mg; 0.17 mmol) in THF (0.5 ml) was treated with N-chlorosuccinimide (23 mg; 0.17 mmol). The mixture was stirred at room temperature for 16 h, concentrated, and the residue purified by flash chromatography eluting with ammonia in MeOH(7N)/CH₂Cl₂ (1/20) to give Example 5 as a pink foam (23 mg).

Yield: 22%

¹H NMR spectrum (CDCl₃): 1.3 (s, 6H); 1.47 (m, 4H); 1.78 (m, 4H); 1.85 (m, 4H); 1.94 (t, 2H); 2.32 (s, 6H); 2.42 (m, 2H); 2.5 (m, 2H); 2.58 (m br, 8H); 2.73 (m, 2H); 3.11 (s, 2H); 3.48 (m, 4H); 4.66 (s br, 2H); 6.90 (s, 1H); 6.99 (s, 2H); 7.83 (s br, 1H).

MS-ESI: 623 and 625 [M+H]⁺

### Example 6

### 3-[3,3-Dimethyl-4-oxo-4-(azabicyclo[2.2.1]heptan-7-yl)butyl]-4-[2-{4-(4-hydroxypiperidin-1-ylcarbonyl)piperidin-1-yl}ethyl]-5-(3,5-dimethylphenyl)-1H -pyrrole

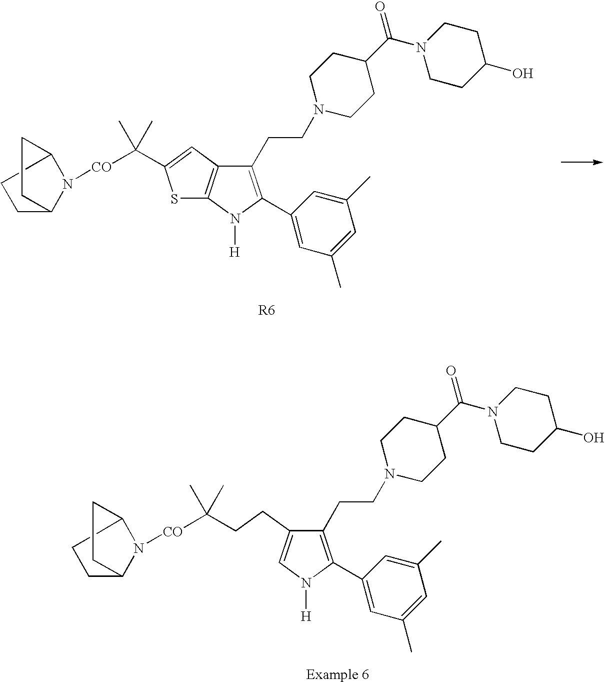

Example 6 was synthesised by the method used for preparing Example 1. The following quantities of starting material and conditions were used: R6 (250 mg; 0.4 mmol); EtOH (140 ml); RaNi (8.9 g); hydrogen (1.5 atm.); 3 hours. Example 6 was obtained as a cream foam (127 mg).

Chromatography: Increasingly polar mixtures of MeOH/CH₂Cl₂ (0-10% MeOH)

Yield: 53%

¹H NMR spectrum (CDCl₃): 1.3 (s, 6H); 1.35-2.2 (m, 19H); 2.34 (s, 6H); 2.4-3.4 (m, 12H); 3.75 (m, 1H); 3.9 (m, 1H); 4.10 (m, 1H); 4.66 (s br, 2H); 6.55 (d, 1H); 6.9 (s, 1H); 7.02 (s, 2H); 7.94 (s, 1H).

MS-ESI: 603 [M+H]⁺

The starting alcohol R6 was prepared as follows:—

### 2-[1,1-Dimethyl-2-oxo-2-azabicyclo[2.2.1]heptan-7-ylethyl]-4-[2-(4-{4-hydroxy piperidin-1-ylcarbonyl}piperidin-1-yl)ethyl]-5-(3,5-dimethylphenyl)-6H -thieno[2,3-b]pyrrole

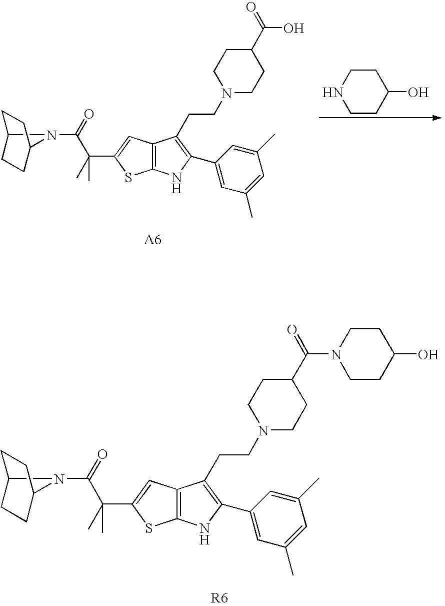

A mixture of A6 (547 mg; 1 mmol), HATU (608 mg; 1.6 mmol) and DIPEA (520 μl; 3 mmol) in CH₂Cl₂ (10 ml) was treated with 4-hydroxypiperidine (202 mg; 2 mmol) and stirred at ambient temperature for 0.5 hours. The mixture was treated with a saturated aqueous solution of NH₄Cl and extracted with CH₂Cl₂. The residue was purified by flash chromatography eluting with increasingly polar mixtures of MeOH/CH₂Cl₂ (0-10% MeOH) to give R6 as a white solid (520 mg).

Yield: 83%

¹H NMR (DMSO d₆): 1.29 (m, 8H); 1.51 (s, 6H); 1.5-1.8 (m, 10H); 2.05 (m, 1H); 2.31 (s, 6H); 2.5 (m, 2H); 2.86 (m, 2H); 2.96 (m, 2H); 3.16 (m, 1H); 3.68 (m, 2H); 3.89 (m, 1H) 4.1 (s br, 1H); 4.55 (s br, 1H); 4.73 (d, 1H); 6.80 (s, 1H); 6.92 (s, 1H); 7.09 (m, 2H).

MS-ESI: 631 [M+H]⁺

The starting material was prepared as follows:—

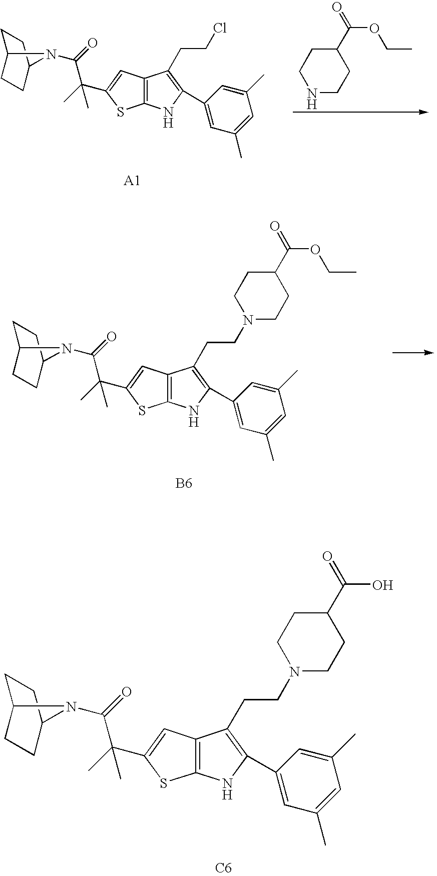

A mixture of A1 (see Example 1) (4.55 g; 0.01 mol), 4-ethoxycarbonyl piperidine (2.36 g; 0.015 mol), triethylamine (1.53 ml; 0.01 μmol) and NaI (1.5 g; 0.01 mol) in DMA (45 ml) was heated at 110° C. under argon atmosphere for 4 hours. After extraction with EtOAc and evaporation, the mixture was purified by flash chromatography, eluting with increasingly polar mixtures of EtOAc/hexanes (80-100% EtOAc) to give B6.

Yield: 62%

¹H NMR (CDCl₃): 1.25 (t, 3H); 1.2-1.45 (m, 4H); 1.5-1.8 (m, 4H); 1.62 (s, 6H); 1.7-2 (m, 4H); 2.05-2.15 (m, 2H); 2.25-2.35 (m, 1H); 2.35 (s, 6H); 2.64-2.67 (m, 2H); 2.93-2.98 (m, 4H); 4.13 (q, 2H); 4.0-4.2 (br m, 1H); 4.6-4.8 (br m, 1H); 6.74 (s, 1H); 6.94 (s, 1H); 7.07 (s, 2H); 8.13 (s, 1H).

A solution of B6 (3.61 g; 0.627 mmol) in 2N NaOH (5 ml) and EtOH (100 ml) was heated at 60° C. for 2 hours. After extraction with CH₂Cl₂/MeOH (1/1) and evaporation, the residue was triturated in ether to give C6 as a solid.

Yield: 93%

¹H NMR (DMSO d₆, AcOH): 1.30 (m, 4H); 1.40-1.70 (m, 4H); 1.53 (s, 6H); 1.80-2.00 (m, 4H); 2.05 (m, 2H); 2.34 (s, 6H); 2.65 (m, br, 1H); 3.14 (m, 2H); 3.27 (m, 2H); 3.30-3.60 (m, 2H); 4.10 (m, br, 1H); 4.50 (m, br, 1H); 6.96 (m, 2H); 7.09 (m, 2H).

### Example 7

### 3-[3,3-Dimethyl-4-oxo-4-(azabicyclo[2.2.1]heptan-7-yl)butyl]-4-[2-{4-(1,1-dioxo-isothiazolidin-2-ylcarbonyl)-4-methoxy-piperidin-1-yl}ethyl]-5-(3,5-dimethylphenyl)-1H-pyrrole

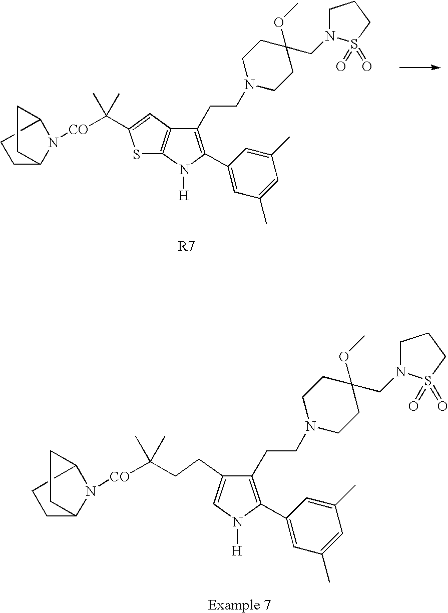

Example 7 was synthesised by the method for preparing Example 1, except that a mixture of MeOH and EtOH was used as solvent. The following quantities of starting material and conditions were used: R7 (65 mg; 0.1 mmol); EtOH (3 ml); MeOH (3 ml); RaNi (5.7 g) hydrogen (1.5 atm.); 2 hours. Example 7 was obtained as a white foam (50 mg).

Chromatography: Increasingly polar mixtures of MeOH/CH₂Cl₂ (0-10% MeOH)

Yield: 81%

¹H NMR spectrum (CDCl₃): 1.29 (s, 6H); 1.47 (m, 4H); 1.62 (s, 4H); 1.79 (m, 4H); 1.87 (m, 4H); 2.34 (s, 6H); 2.39 (m, 2H); 2.44 (m, 2H), 2.51 (m, 2H); 2.65 (m, 2H); 2.77 (m, 2H); 3.06 (s br, 2H); 3.1 (t, 2H); 3.22 (s, 3H); 3.42 (t, 2H); 4.66 (s br, 2H); 6.55 (d, 1H); 6.9 (s, 1H); 7.02 (s, 2H); 7.93 (s, 1H).

MS-ESI: 639 [M+H]⁺

R7 was prepared as follows:—

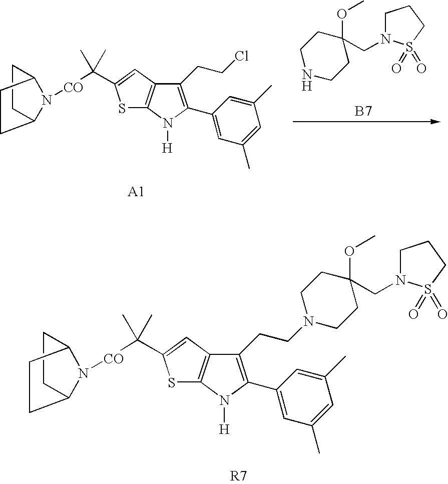

A solution of A1 (see Example 1) (260 mg; 0.57 mmol) and B7 (325 mg; 1.15 mmol) in DMF (1.5 ml) and acetonitrile (3 ml) under argon was treated with K₂CO₃ (239 mg; 1.72 mmol). The mixture was heated at 90° C. for 3 hours. The mixture was cooled, treated with pentane and filtered. The residue was purified by flash chromatography eluting with MeOH/CH₂Cl₂ (5% MeOH) to give R7 as a white foam (260 mg).

Yield: 68%

¹H NMR spectrum (CDCl₃): 1.32 (s br, 4H); 1.62 (s, 4H); 1.88 (m, 2H); 2.35 (s, 6H); 2.41 (m, 2H); 2.68 (m, 4H); 2.95 (m, 2H); 3.06 (s, 2H); 3.1 (t, 2H); 3.22 (s, 3H); 3.43 (t, 2H); 4.10 (s br, 1H); 4.7 (s br, 1H); 6.75 (s, 1H); 6.94 (s, 1H); 7.06 (s, 2H); 8.13 (s, 1H).

MS-ESI: 667 [M+H]⁺

B7 was prepared as follows:—

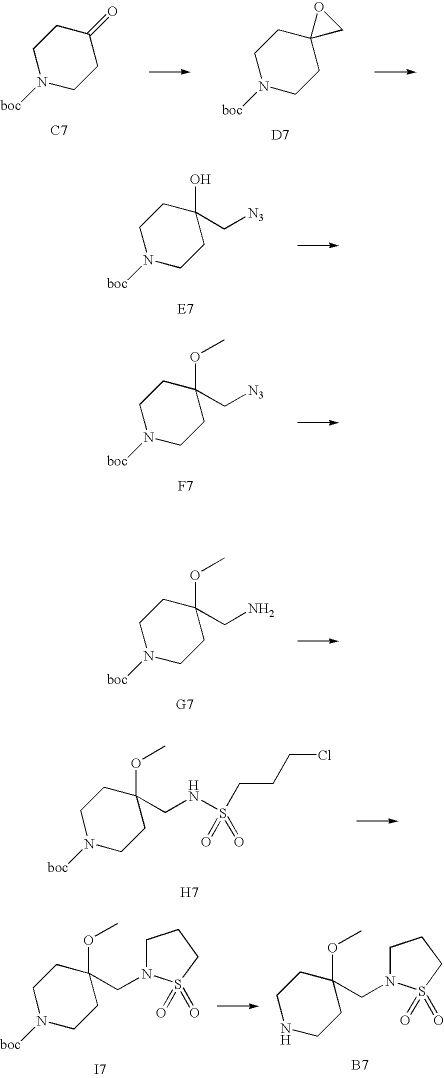

A 60% suspension of NaH in oil (230 mg; 5.5 mmol) in DMSO (6 ml) under argon was treated with trimethylsulphonium iodide (1.26 g; 5.75 mmol) at 5° C. The mixture was stirred for 30 minutes and C7 (1.0 g; 5 mmol) was added. The mixture was stirred for 1 hour at room temperature. The mixture was partitioned between water and diethyl ether. The diethyl ether was evaporated and the residue purified by flash chromatography eluting with increasingly polar mixtures of EtOAc/CH₂Cl₂ (0-30% EtOAc) to give D7 (870 mg).

Yield: 82%

¹H NMR spectrum (CDCl₃): 1.47 (s, 9H); 1.50 (m, 2H); 1.78 (m, 2H); 2.69 (s, 2H); 3.43 (m, 2H); 3.71 (m, 2H).

A solution of D7 (3.9 g; 18.3 mmol) in MeOH (100 ml) and water (20 ml) was treated with sodium azide (5.95 g; 91.5 mmol) and ammonium chloride (1.96 g; 36.6 mmol). The mixture was heated under reflux for 15 hours. The solvents were removed by evaporation and the residue partitioned between water and CH₂Cl₂. The CH₂Cl₂ was evaporated and the residue purified by flash chromatography eluting with increasingly polar mixtures of EtOAc/hexanes (20-30% EtOAc) to give E7 (4.24 g).

Yield: 90%

¹H NMR spectrum (CDCl₃): 1.46 (s, 9H); 1.50 (m, 2H); 1.62 (m, 2H); 1.79 (s, 1H); 3.14 (m, 2H); 3.3 (s, 2H); 3.85 (m, 2H).

A solution of E7 (4.2 g; 16.4 mmol) in THF (20 ml) under argon was treated with a 60% suspension of NaH in oil (868 mg; 18.9 mmol). Methyl iodide (1.23 ml; 19.7 mmol) was added dropwise followed by crown ether 15-5 (30 drops). The mixture was stirred overnight, the THF evaporated and the residue partitioned between water and EtOAc. The residue was purified by flash chromatography with EtOAc/hexanes (20% EtOAc) to give F7 (4.15 g).

Yield: 94%

¹H NMR spectrum (CDCl₃): 1.42 (m, 2H); 1.46 (s, 9H); 1.81 (m, 2H); 3.1 (m, 2H); 3.25 (m, 5H); 3.8 (m, 2H).

A solution of F7 (3.45 g; 12.9 mmol) in EtOH (100 ml) and EtOAc (10 ml) was hydrogenated (1.2 atm. H₂) over Pd/C (520 mg; 10%) for 2.5 hours. The mixture was filtered to give G7 (2.94 g).

Yield: 93%

¹H NMR spectrum (CDCl₃): 1.35 (m, 2H); 1.46 (s, 9H); 1.76 (m, 2H); 2.66 (s, 2H); 3.1 (m, 2H); 3.19 (s, 3H); 3.8 (m, 2H).

A solution of G7 (750 mg; 3.07 mmol) in EtOAc (25 ml) was cooled to 0° C. and treated successively with K₂CO₃ (470 mg; 3.38 mmol) and 3-chloropropanesulphonyl chloride (411 μl; 3.38 mmol). The mixture was allowed to warm to room temperature overnight and the solvent evaporated. The residue was purified by flash chromatography eluting with ammonia in MeOH(7N)/CH₂Cl₂ (1/20) to give H7 (1.02 g).

Yield: 86%

¹H NMR spectrum (CDCl₃): 1.43 (m, 2H); 1.45 (s, 9H); 1.82 (m, 2H); 2.29 (m, 2H); 3.15 (m, 4H); 3.2 (s, 3H); 3.23 (m, 2H); 3.69 (t, 2H); 3.8 (m, 2H); 4.41 (t, 1H).

A solution of H7 (1.0 g; 2.6 mmol) in toluene (35 ml) was cooled to 0° C. A 60% suspension of NaH in oil (156 mg; 3.9 mmol) was added and the mixture heated at 90° overnight. The solvent was evaporated and the residue purified by flash chromatography eluting with ammonia in MeOH(7N)/CH₂Cl₂ (1/20) to give I7 (910 mg).

Yield: 99%

¹H NMR spectrum (CDCl₃): 1.45 (s, 9H); 1.49 (m, 2H); 1.79 (m, 2H); 2.36 (m, 2H); 3.05 (s, 2H); 3.11 (m, 4H); 3.23 (s, 3H); 3.4 (m, 2H); 3.75 (t, 2H).

A solution of I7 (910 mg; 2.61 mmol) in 1,4-dioxane (7 ml) and CH₂Cl₂ (1 ml) was treated with a mixture of 1,4-dioxane (3.3 ml) and conc. HCl (0.7 ml). The mixture was stirred overnight and the solvents removed to give B7 as a white solid (745 mg).

Yield: 99%

¹H NMR spectrum (DMSO d₆): 1.61 (m, 2H); 1.90 (m, 2H); 2.23 (m, 2H); 2.88 (m, 2H); 3.03 (s, 2H); 3.12 (m, 4H); 3.15 (s, 3H); 3.33 (2H).

MS-ESI: 249 [M+H]⁺

### Example 8

### 3-[3,3-Dimethyl-4-oxo-4-(azabicyclo[2.2.1]heptan-7-yl)butyl]-4-[1S-methyl-2-{1-benzyl-pyrrodin-3-ylamino}ethyl]-5-(3,5-dimethylphenyl)-1H-pyrrole

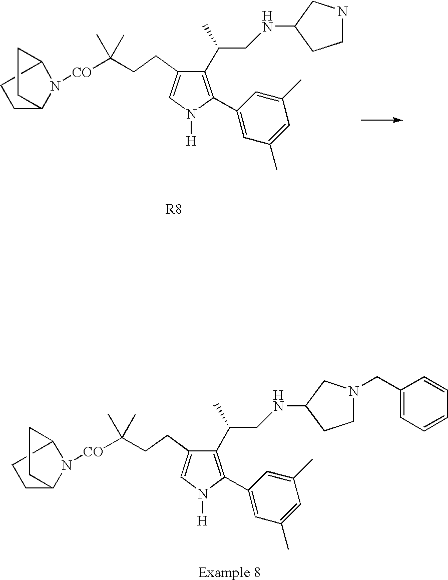

A solution of R8 (100 mg 0.2 mmol) in MeOH (3 ml) was treated with benzaldehyde (22 μl; 0.22 mmol) and three drops of acetic acid. The mixture was cooled to 0° C. Sodium cyanoborohydride (14 mg; 0.22 mmol) was added and the mixture was allowed to warm to room temperature for 2 hours. A saturated aqueous solution of NaHCO₃ was added, the MeOH evaporated and the mixture extracted into CH₂Cl₂. The solvent was evaporated and the residue purified by flash chromatography eluting with increasingly polar mixtures of MeOH/CH₂Cl₂ (0-10% MeOH) to give Example 8 as a rose foam (76 mg).

Yield: 64%

¹H NMR spectrum (CDCl₃): 1.3 (m, 9H); 1.46 (m, 4H); 1.77 (m, 1H); 1.78 (m, 4H); 1.88 (m, 2H); 1.91 (m, 1H); 2.29 (m, 1H); 2.31 (d, 6H); 2.33-2.81 (m, 7H); 3.18 (m, 2H); 3.6 (m, 2H); 4.66 (s br, 2H); 6.56 (s, 1H); 6.91 (s, 1H); 6.97 (d, 2H); 7.3 (s, 5H); 7.83 (s br, 1H).

MS-ESI: 581 [M+H]⁺

The starting material was prepared as follows:—

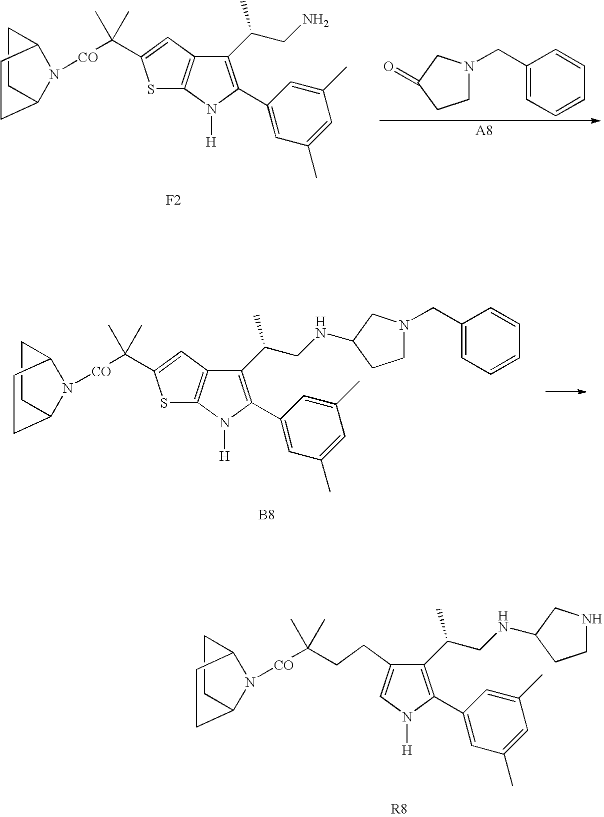

A solution of F2 (300 mg; 0.67 mmol) in MeOH (2 ml) was treated with N-benzyl-3-pyrrolidone (120 μl; 0.73 mmol) and acetic acid (5 drops). Sodium cyanoborohydride (46 mg; 0.73 mmol) was added and the mixture stirred overnight. A saturated aqueous solution of NaHCO₃ was added and the MeOH evaporated. The mixture was extracted into CH₂Cl₂. The solvent was evaporated and the residue purified by flash chromatography eluting with increasingly polar mixtures of MeOH/CH₂Cl₂ (0-10% MeOH) to give B8 as a faint rose foam (250 mg).

Yield: 62%

¹H NMR spectrum (CDCl₃): 1.31 (d, 3H); 1.4 (m, 4H); 1.53 (m, 1H); 1.61 (t, 6H); 1.65 (m, 4H); 1.99 (m, 1H); 2.21 (m, 1H); 2.33 (d, 6H); 2.5 (m, 2H); 2.7-2.9 (m, 3H); 3.22 (m, 2H); 3.57 (m, 2H); 4.1 (s br, 1H); 4.75 (s br, 1H); 6.75 (d, 1H); 6.94 (s, 1H); 7.08 (s, 2H); 7.25 (m, 5H); 8.12 (s, 1H).

MS-ESI: 609 [M+H]⁺

A suspension of B8 (215 mg; 0.35 mmol) in EtOH (5 ml) and MeOH (5 ml) was treated with Raney-Nickel (10 g) and placed under an atmosphere of hydrogen (1.7 atm.). The mixture was stirred at room temperature for 2 hours. The mixture was filtered, the filtrate concentrated and the residue purified by flash chromatography eluting with increasingly polar mixtures of ammonia in MeOH(7N)/CH₂Cl₂ (0-10% MeOH) to give R8 as a white foam (100 mg).

Yield: 58%

¹H NMR spectrum (CDCl₃): 1.26 (d, 3H); 1.29 (s, 6H); 1.47 (m, 4H); 1.79 (m, 4H); 1.8-1.9 (m, 4H); 2.33 (d, 6H); 2.51 (m, 2H); 2.65-3.15 (m, 8H); 4.67 (s br, 2H); 6.56 (d, 1H); 6.94 (s, 1H); 7.0 (s, 2H); 7.85 (s br, 1H).

MS-ESI: 491 [M+H]⁺

### Example 9

### 3-[3,3-Dimethyl-4-oxo-4-(azabicyclo[2.2.1]heptan-7-yl)butyl]-4-[1S-methyl-2-(2-{4-N-isopropylureidophenyl}ethylamino)ethyl]-5-(3,5-dimethylphenyl)-1H-pyrrole

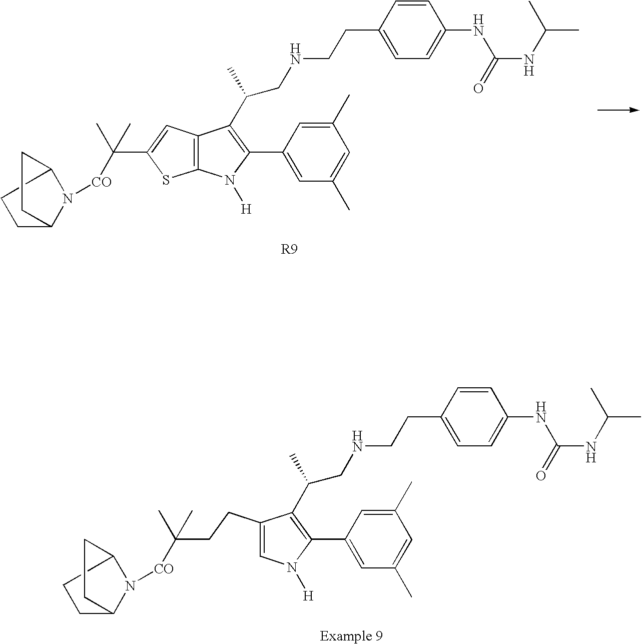

Example 9 was synthesised by the method used for preparing Example 1, except that a mixture of MeOH and EtOH was used as solvent. The following quantities of starting material and conditions were used: R9 (40 mg; 0.06 mmol); EtOH (3 ml); MeOH (3 ml); RaNi (1.5 g); 16 hours. Example 9 was obtained as a beige foam (20 mg).

Chromatography: MeOH/CH₂Cl₂ (0-10% MeOH)

Yield: 53%

¹H NMR spectrum (CDCl₃): 1.18 (m, 6H); 1.28 (m, 6H); 1.32 (d, 3H); 1.45 (m, 4H); 1.76; (m, 4H); 1.85 (m, 2H); 2.28 (s, 6H); 2.4 (t, 2H); 2.64 (m, 1H); 2.77 (m, 2H); 2.87 (t, 1H) 2.9 (m, 1H); 3.05 (m, 1H); 3.32 (m, 1H); 3.95 (m, 1H); 4.62 (s br, 2H); 5.8 (s br, 1H); 6.54 (d, 1H); 6.74 (d, 2H); 6.77 (s, 2H); 6.88 (s, 1H); 7.22 (d, 2H); 8.1 (s br, 1H); 8.69 (s, 1H).

MS-ESI: 626 [M+H]⁺

The starting material was prepared as follows:—

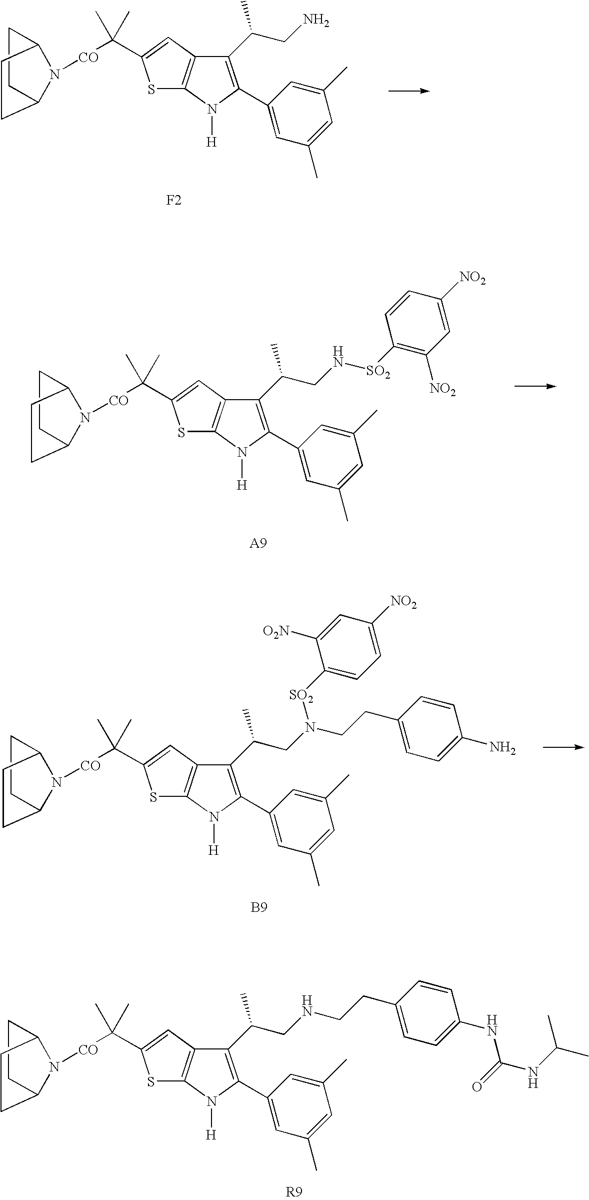

A solution of F2 (1.5 g; 3.34 mmol) in CH₂Cl₂ (20 ml) under argon at 0° C. was treated with collidine (485 μl; 3.67 mmol). 2,4-Dinitrophenyl sulphonyl chloride (978 mg; 3.67 mmol) was added. The mixture was stirred for 1 hours and evaporated. The residue was purified by flash chromatography with EtOAc/CH₂Cl₂ (20% EtOAc) to give A9 as a brown powder (1.9 g).

Yield: 84%.

¹H NMR spectrum (CDCl₃): 1.31 (m, 4H); 1.33 (d, 3H); 1.62 (s, 6H); 1.65 (m, 4H); 2.27 (s, 6H); 2.91 (m, 1H); 3.55 (m, 1H); 3.65 (m, 1H); 4.1 (s br, 1H); 4.7 (s br, 1H); 5.34 (s, 1H); 6.69 (s, 1H); 6.72 (s, 2H); 6.81 (s, 1H); 8.12 (d, 1H); 8.19 (s, 1H); 8.22 (d, 1H); 8.33 (dd, 1H).

A solution of A9 (130 mg; 0.19 mmol) and 3-(2-hydroxyethyl)aniline (40 mg; 0.29 mmol) in THF (10 ml) was cooled to 0° C. Ph₃P (301 mg; 1.15 mmol) and then DTAD (177 mg; 0.77 mmol) were added and the mixture was stirred for 2 hours. Water was added, the THF was evaporated and the residue partitioned between water and CH₂Cl₂. The residue was purified by flash chromatography with increasingly polar solutions of EtOAc/CH₂Cl₂ (0-20% EtOAc) to give crude B9 (530 mg) contaminated with Ph₃O.

MS-ESI: 800 [M+H]⁺

A solution of crude B9 (530 mg) in CH₂Cl₂ (10 ml) was treated with 2-propyl isocyanate (332 μl; 3.4 mmol). The mixture was heated at 45° C. for 2 h, cooled and partitioned between water and CH₂Cl₂. The residue was taken up in n-propylamine (5 ml), stirred for 1 hour and evaporated. The residue was purified by flash chromatography with increasingly polar solutions of EtOAc/CH₂Cl₂ (0-100% EtOAc) and then MeOH/CH₂Cl₂ (0-10% MeOH) to give R9 (60 mg).

Yield: 49%, for the previous two steps.

¹H NMR spectrum (CDCl₃): 1.19 (m, 6H); 1.31 (d, 3H); 1.32 (m, 4H); 1.63 (s, 6H); 1.65 (m, 4H); 2.32 (s, 6H); 2.55 (m, 3H); 2.8 (m, 2H); 3.03 (m, 1H); 3.27 (m, 1H); 3.99 (m, 1H); 4.1 (s br, 1H); 4.7 (s br, 1H); 5.34 (s, 1H); 6.61 (d, 2H); 6.64 (s, 1H); 6.89 (s, 1H); 6.94 (s, 2H); 7.05 (d, 2H); 7.5 (s br, 1H); 9.6 (s, 1H).

MS-ESI: 654 [M+H]⁺

### Example 10

### 3-[3,3-Dimethyl-4-oxo-4-(azabicyclo[2.2.1]heptan-7-yl)butyl]-4-[1S-methyl-2-{4-(pyrid-4-yl)piperidin-1-ylcarbonylamino}ethyl]-5-(3,5-dimethylphenyl)-1H-pyrrole

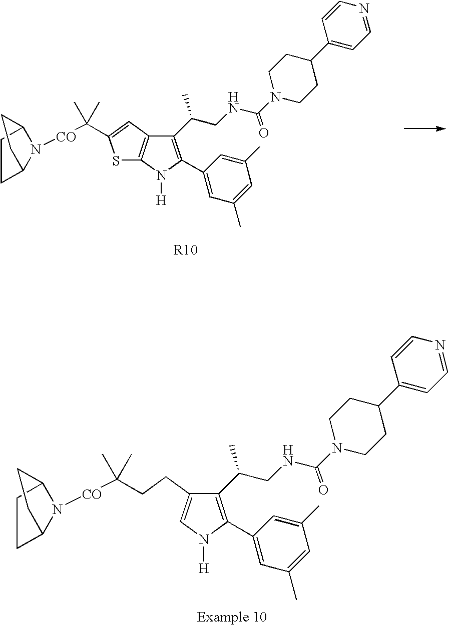

Example 10 was synthesised by the method used for preparing Example 1, except that a mixture of MeOH and EtOH was used as solvent. The following quantities of starting material and conditions were used: R10 (190 mg; 0.3 mmol); EtOH (5 ml); MeOH (5 ml); RaNi (2 g); hydrogen (1.5 atm.); 16 hours. Example 10 was obtained as a cream foam (80 mg).

Chromatography: MeOH/CH₂Cl₂ (0-5% MeOH)

Yield: 44%

¹H NMR spectrum (CDCl₃): 1.3 (m, 9H); 1.46 (m, 4H); 1.74 (m, 8H); 1.9 (m, 2H); 2.31 (s, 6H); 2.55 (m, 3H); 2.72 (m, 2H); 3.21 (m, 2H); 3.64 (m, 1H); 3.80 (m, 1H); 3.95 (m, 1H); 4.44 (d, 1H); 4.66 (s br, 2H); 6.59 (d, 1H); 6.92 (s, 1H); 6.98 (s, 2H); 7.05 (d, 2H); 7.89 (s, 1H); 8.50 (d, 2H).

MS-ESI: 610 [M+H]⁺

The starting material was prepared as follows:—

### 2-(1,1-dimethyl-2-oxo-2-azabicyclo[2.2.1]heptan-7-ylethyl)-4-[1S-methyl-2-(3-{pyridin-4-yl}piperidin-1-yl)carbonylamino-ethyl]-5-(3,5-dimethylphenyl)-6H-thieno[2,3-b]pyrrole

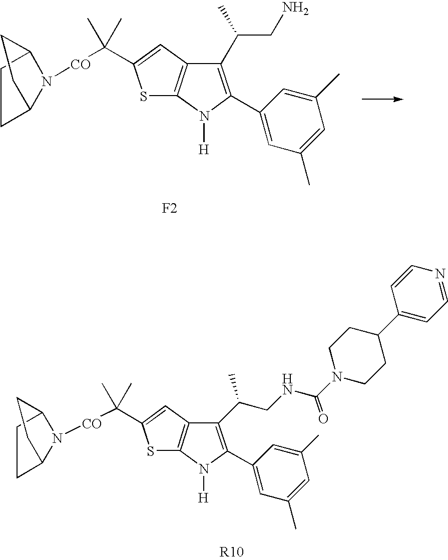

4-Nitrophenyl chloroformate (183 mg; 0.91 mmol) was added under an argon atmosphere, at 0° C., to a solution of F2 (370 mg; 0.82 mmol) and DIPEA (287 μl; 1.65 mmol) in CH₂Cl₂ (10 ml). The mixture was stirred at 0° C. for 30 minutes. 4-Piperidin-4-yl pyridine (186 mg; 1.15 mmol) was added. The mixture was stirred for 16 hours and was purified by flash chromatography with increasingly polar solutions of EtOAc/CH₂Cl₂ (0-100% EtOAc) and then MeOH/CH₂Cl₂ (0-15% MeOH) to give R10 as a pale yellow solid (190 mg).

Yield: 36%

¹H NMR spectrum (CDCl₃): 1.32 (m, 4H); 1.36 (d, 3H); 1.63 (s, 6H); 1.6-1.8 (m, 8H); 2.33 (s, 6H); 2.58 (m, 1H); 2.70 (m, 2H); 3.24 (m, 1H); 3.32 (m, 1H); 3.75 (m, 2H); 3.96 (d, 1H); 4.12 (s br, 1H); 4.39 (m, 1H); 4.7 (s br, 1H); 6.79 (s, 1H); 6.94 (s, 1H); 7.04 (d, 2H); 7.07 (s, 2H); 8.26 (s, 1H); 8.49 (m, 2H).

MS-ESI: 638 [M+H]⁺

### Example 11

### 3-[3,3-Dimethyl-4-oxo-4-(azabicyclo[2.2.1]heptan-7-yl)butyl]-4-[1S-methyl-2-{3-(pyrid-4-yl)pyrrolidin-1-ylcarbonylamino}ethyl]-5-(3,5-dimethylphenyl)-1H-pyrrole

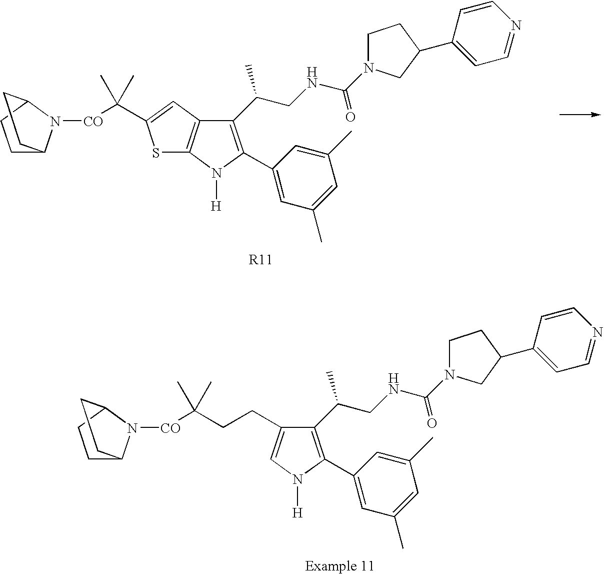

Example 11 was synthesised by the method used for preparing Example 1, except that a mixture of MeOH and EtOH was used as solvent. The following quantities of starting material and conditions were used: R11 (367 mg; 0.59 mmol); EtOH (5 ml); MeOH (5 ml); RaNi (10 g); hydrogen (1.5 atm.); 20 hours. Example 11 was obtained as a white foam (31 mg).

Chromatography: MeOH/CH₂Cl₂ (0-10% MeOH)

Yield: 9%

¹H NMR spectrum (CDCl₃): 1.29 (m, 9H); 1.46 (m, 4H); 1.78 (m, 4H); 1.91 (m, 3H); 2.3 (m, 1H); 2.31 (s, 6H); 2.53 (m, 2H); 3.15-3.45 (m, 5H); 3.65 (m, 2H); 4.20 (m, 1H); 4.66 (s br, 2H); 6.59 (d, 1H); 6.92 (s, 1H); 6.96 (s, 2H); 7.1 (m, 2H); 7.9 (s, 1H); 8.51 (s, 2H).

MS-ESI: 596 [M+H]⁺

R11 was prepared using a method analogous to R10 (see Example 10)

¹H NMR spectrum (CDCl₃): 1.28 (m, 4H); 1.37 (d, 3H); 1.62 (s, 6H); 1.71 (m, 4H); 1.9 (m, 1H); 2.25 (m, 1H); 2.31 (s, 6H); 3.1-3.3 (m, 6H); 3.6 (m, 1H); 3.74 (m, 1H); 4.13 (s br, 1H); 4.14 (m, 1H); 4.7 (s br, 1H); 6.79 (s, 1H); 6.93 (m, 1H); 7.05 (m, 4H); 8.36 (s, 1H); 8.50 (m, 2H).

MS-ESI: 624 [M+H]⁺

### Example 12

### 3-[3,3-Dimethyl-4-oxo-4-(azabicyclo[2.2.1]heptan-7-yl)butyl]-4-[1S-methyl-2-{4-phenylpiperidin-1-ylcarbonylamino}ethyl]-5-(3,5-dimethylphenyl)-1H-pyrrole

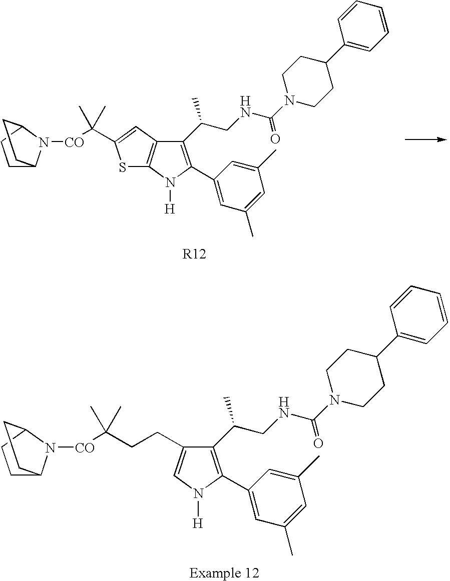

Example 12 was synthesised by the method used for preparing Example 1, except that a mixture of MeOH and EtOH was used as solvent. The following quantities of starting material and conditions were used: R12 (435 mg; 0.68 mmol); EtOH (10 ml); MeOH (10 ml); RaNi (5 g); 16 hours. Example 12 was obtained as a yellow foam (258 mg).

Chromatography: EtOAc/hexanes (0-100% EtOAc)

Yield: 62%

¹H NMR spectrum (CDCl₃): 1.29 (m, 9H); 1.46 (m, 4H); 1.75 (m, 8H); 1.91 (m, 2H); 2.31 (s, 6H); 2.55 (m, 3H); 2.71 (m, 2H); 3.21 (m, 2H); 3.63 (m, 1H); 3.80 (m, 1H); 3.91 (m, 1H); 4.42 (d, 1H); 4.66 (s br, 2H); 6.59 (d, 1H); 6.92 (s, 1H); 6.98 (s, 2H); 7.12 (d, 2H); 7.20 (t, 1H); 7.28 (t, 2H); 7.88 (s, 1H).

MS-ESI: 609 [M+H]⁺

R12 was prepared in the same manner as R10 (see Example 10)

¹H NMR spectrum (CDCl₃): 1.32 (m, 4H); 1.36 (d, 3H); 1.63 (s, 6H); 1.6-1.8 (m, 8H); 2.33 (s, 6H); 2.55 (m, 1H); 2.68 (m, 2H); 3.22 (m, 1H); 3.3 (m, 1H); 3.75 (m, 2H); 3.9 (d, 1H); 4.13 (s br, 1H); 4.38 (m, 1H); 4.7 (s br, 1H); 6.80 (s, 1H); 6.94 (m, 1H); 7.07 (s, 2H); 7.11 (d, 2H); 7.20 (t, 1H); 7.28 (m, 2H); 8.29 (s, 1H).

MS-ESI: 637 [M+H]⁺

Therapeutic Uses

Compounds of Formula (I) are provided as medicaments for antagonising gonadotropin releasing hormone (GnRH) activity in a patient, eg, in men and/or women. To this end, a compound of Formula (I) can be provided as part of a pharmaceutical formulation which also includes a pharmaceutically acceptable diluent or carrier (eg, water). The formulation may be in the form of tablets, capsules, granules, powders, syrups, emulsions (eg, lipid emulsions), suppositories, ointments, creams, drops, suspensions (eg, aqueous or oily suspensions) or solutions (eg, aqueous or oily solutions). If desired, the formulation may include one or more additional substances independently selected from stabilising agents, wetting agents, emulsifying agents, buffers, lactose, sialic acid, magnesium stearate, terra alba, sucrose, corn starch, talc, gelatin, agar, pectin, peanut oil, olive oil, cacao butter and ethylene glycol.

The compound is preferably orally administered to a patient, but other routes of administration are possible, such as parenteral or rectal administration. For intravenous, subcutaneous or intramuscular administration, the patient may receive a daily dose of 0.1 mgkg⁻¹ to 30 mgkg⁻¹ (preferably, 5 mgkg⁻¹ to 20 mgkg⁻¹) of the compound, the compound being administered 1 to 4 times per day. The intravenous, subcutaneous and intramuscular dose may be given by means of a bolus injection. Alternatively, the intravenous dose may be given by continuous infusion over a period of time. Alternatively, the patient may receive a daily oral dose which is approximately equivalent to the daily parenteral dose, the composition being administered 1 to 4 times per day. A suitable pharmaceutical formulation is one suitable for oral administration in unit dosage form, for example as a tablet or capsule, which contains between 1 0 mg and 1 g (preferably, 100 mg and 1 g) of the compound of the invention.

Buffers, pharmaceutically acceptable co-solvents (eg, polyethylene glycol, propylene glycol, glycerol or EtOH) or complexing agents such as hydroxy-propyl p cyclodextrin may be used to aid formulation.

One aspect of the invention relates to the use of compounds according to the invention for reducing the secretion of LH and/or FSH by the pituitary gland of a patient. In this respect, the reduction may be by way of a reduction in biosynthesis of the LH and FSH and/or a reduction in the release of LH and FSH by the pituitary gland. Thus, compounds according to the invention can be used for therapeutically treating and/or preventing a sex hormone related condition in the patient. By “preventing” we mean reducing the patient's risk of contracting the condition. By “treating” we mean eradicating the condition or reducing its severity in the patient. Examples of sex hormone related conditions are: a sex hormone dependent cancer, benign prostatic hypertrophy, myoma of the uterus, endometriosis, polycystic ovarian disease, uterine fibroids, prostatauxe, myoma uteri, hirsutism and precocious puberty. Examples of sex hormone dependent cancers are: prostatic cancer, uterine cancer, breast cancer and pituitary gonadotrophe adenoma.

The compounds of the invention may be used in combination with other drugs and therapies used to treat/prevent sex-hormone related conditions.

If formulated as a fixed dose such combination products employ the compounds of this invention within the dosage range described herein and the other pharmaceutically-active agent within its approved dosage range. Sequential use is contemplated when a combination formulation is inappropriate.

In the field of medical oncology examples of such combinations include combinations with the following categories of therapeutic agent:

i) anti-angiogenic agents (for example linomide, inhibitors of integrin αvβ3 function, angiostatin, endostatin, razoxin, thalidomide) and including vascular endothelial growth factor (VEGF) receptor tyrosine kinase inhibitors (RTKIs) (for example those described in international patent applications publication nos. WO-97/22596, WO-97/30035, WO-97/32856 and WO-98/13354, the entire disclosure of which documents is incorporated herein by reference); ii) cytostatic agents such as anti-oestrogens (for example tamoxifen, toremifene, raloxifene, droloxifene, iodoxyfene), progestogens (for example megestrol acetate), aromatase inhibitors (for example anastrozole, letrozole, vorazole, exemestane), anti-progestogens, anti-androgens (for example flutamide, nilutamide, bicalutamide, cyproterone acetate), inhibitors of testosterone 5α-dihydroreductase (for example finasteride), anti-invasion agents (for example metalloproteinase inhibitors like marimastat and inhibitors of urokinase plasminogen activator receptor function) and inhibitors of growth factor function, (such growth factors include for example epidermal growth factor (EGF), platelet derived growth factor and hepatocyte growth factor such inhibitors include growth factor antibodies, growth factor receptor antibodies, tyrosine kinase inhibitors and serine/threonine kinase inhibitors);

iii) biological response modifiers (for example interferon);

iv) antibodies (for example edrecolomab); and

v) anti-proliferative/anti-neoplastic drugs and combinations thereof, as used in medical oncology, such as anti-metabolites (for example anti-foliates like methotrexate, fluoropyrimidines like 5-fluorouracil, purine and adenosine analogues, cytosine arabinoside); anti-tumour antibiotics (for example anthracyclines like doxorubicin, daunomycin, epirubicin and idarubicin, mitomycin-C, dactinomycin, mithramycin); platinum derivatives (for example cisplatin, carboplatin); alkylating agents (for example nitrogen mustard, melphalan, chlorambucil, busulphan, cyclophosphamide, ifosfamide, nitrosoureas, thiotepa); anti-mitotic agents (for example vinca alkaloids like vincristine and taxoids like taxol, taxotere); enzymes (for example asparaginase); thymidylate synthase inhibitors (for example raltitrexed); topoisomerase inhibitors (for example epipodophyllotoxins like etoposide and teniposide, amsacrine, topotecan, irinotecan).

The compounds of the invention may also be used in combination with surgery or radiotherapy.

Assays

The ability of compounds according to the invention to act as antagonists of GnRH can be determined using the following in vitro assays.

Binding Assay Using Rat Pituitary GnRH Receptor

The assay is performed as follows:—
1. Incubate crude plasma membranes prepared from rat pituitary tissues in a Tris.HCl buffer (pH. 7.5, 50 mM) containing bovine serum albumin (0.1%), [I-125]D-t-Bu-Ser6-Pro9-ethyl amide-GnRH, and the test compound. Incubation is at 4° C. for 90 minutes to 2 hours.
2. Rapidly filter and repeatedly wash through a glass fibre filter.
3. Determine the radioactivity of membrane bound radio-ligands using a gamma counter.

From this data, the IC₅₀ of the test compound can be determined as the concentration of the compound required to inhibit radio-ligand binding to GnRH receptors by 50%. Compounds according to the present invention have activity at a concentration from 1 nM to 5 μM.

Binding Assay Using Human GnRH Receptor

Crude membranes prepared from CHO cells expressing human GnRH receptors are sources for the GnRH receptor. The binding activity of compounds according to the invention can be determined as an IC₅₀ which is the compound concentration required to inhibit the specific binding of [¹²⁵I]buserelin to GnRH receptors by 50%. [¹²⁵I]Buserelin (a peptide GnRH analogue) is used here as a radiolabelled ligand of the receptor.

Assay to Determine Inhibition of LH Release

The LH release assay can be used to demonstrate antagonist activity of compounds, as demonstrated by a reduction in GnRH-induced LH release.

Preparation of Pituitary Glands

Pituitary glands obtained from rats are prepared as follows. Suitable rats are Wistar male rats (150-200 g) which have been maintained at a constant temperature (eg, 25° C.) on a 12 hour light/12 hour dark cycle. The rats are sacrificed by decapitation before the pituitary glands are aseptically removed to tube containing Hank's Balanced Salt Solution (HBSS). The glands are further processed by:—
1. Centrifugation at 250×g for 5 minutes;
2. Aspiration of the HBSS solution;
3. Transfer of the glands to a petri dish before mincing with a scalpel;
4. Transfer of the minced tissue to a centrifuge tube by suspending the tissue three successive times in 10 ml aliquots of HBSS containing 0.2% collagenase and 0.2% hyaluronidase;
5. Cell dispersion by gentle stirring of the tissue suspension while the tube is kept in a water bath at 37° C.;
6. Aspiration 20 to 30 times using a pipette, undigested pituitary fragments being allowed to settle for 3 to 5 minutes;
7. Aspiration of the suspended cells followed by centrifugation at 1200×g for 5 minutes;
8. Re-suspension of the cells in culture medium of DMEM containing 0.37% NaHCO₃, 10% horse serum, 2.5% foetal bovine serum, 1% non essential amino acids, 1% glutamine and 0.1% gentamycin;
9. Treatment of the undigested pituitary fragments 3 times with 30 ml aliquots of the collagenase and hyaluronidase;
10. Pooling of the cell suspensions and dilution to a concentration of 3×10⁵ cells/ml;
11. Placing of 1.0 ml of this suspension in each of a 24 well tray, with the cells being maintained in a humidified 5% CO₂/95% air atmosphere at 37° C. for 3 to 4 days
   Testing of Compounds

The test compound is dissolved in DMSO to a final concentration of 0.5% in the incubation medium.

1.5 hours prior to the assay, the cells are washed three times with DMEM containing 0.37% NaHCO₃, 10% horse serum, 2.5% foetal bovine serum, 1% non essential amino acids (100×), 1% glutamine (100×), 1% penicillin/streptomycin (10,000 units of each per ml) and 25 mM HEPES at pH 7.4. Immediately prior to the assay, the cells are again washed twice in this medium.

Following this, 1 ml of fresh medium containing the test compound and 2 nM GnRH is added to two wells. For other test compounds (where it is desired to test more than one compound), these are added to other respective duplicate wells. Incubation is then carried out at 37° C. for three hours.

Following incubation, each well is analysed by removing the medium from the well and centrifuging the medium at 2000×g for 15 minutes to remove any cellular material. The supernatant is removed and assayed for LH content using a double antibody radio-immuno assay. Comparison with a suitable control (no test compound) is used to determine whether the test compound reduces LH release. Compounds according to the present invention have activity at a concentration from 1 nM to 5 μM.

1. A method of antagonising gonadotropin releasing hormone activity in a patient, comprising administering a compound of formula (I): 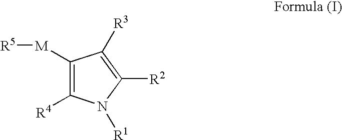
wherein:
R¹ is selected from: hydrogen, optionally substituted C₁₋₆alkyl, optionally substituted aryl or optionally substituted arylC₁₋₆alkyl, wherein the optional substituents are selected from C₁₋₄alkyl, nitro, cyano, fluoro and C₁₋₄alkoxy;
R² is an optionally substituted mono or bi-cyclic aromatic ring, wherein the optional substituents are 1, 2 or 3 substituents independently selected from: cyano, R^{e}R^{f}N—, C₁₋₆alkyl, C₁₋₆alkoxy, halo, haloC₁₋₆alkyl or haloC₁₋₆alkoxy wherein R^{e} and R^{f} are independently selected from hydrogen, C₁₋₆alkyl or aryl;
R³ is selected from a group of Formula (IIa) to Formula (IId): 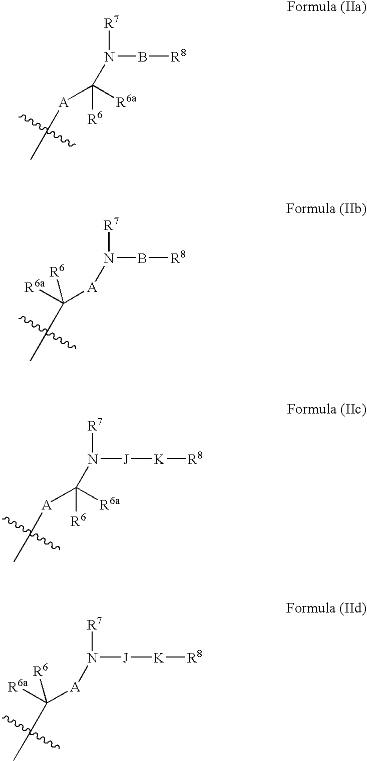
where R⁶ and R^{6a} are independently selected from hydrogen, fluoro, optionally substituted C₁₋₆alkyl, C₁₋₆alkoxy, or R⁶ and R^{6a} taken together and the carbon atom to which they are attached form a carbocyclic ring of 3-7 atoms or R⁶ and R^{6a} taken together and the carbon atom to which they are attached form a carbonyl group;
or when A is not a direct bond the group 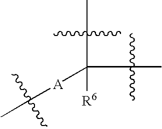
forms a carbocyclic ring of 3-7 carbon atoms or a heterocyclic ring containing one or more heteroatoms;
or the group 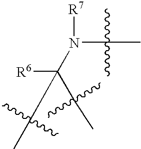
forms a heterocyclic ring containing 3-7 carbon atoms and one or more heteroatoms;
R⁷ is selected from: hydrogen or C₁₋₆alkyl;
R⁸ is selected from:
(i) hydrogen, C₁₋₆alkyl, C₂₋₆alkenyl, C₂₋₆alkynyl, haloC₁₋₆alkyl, C₁₋₄alkoxyC₁₋₄alkyl, hydroxy, hydroxyC₁₋₆alkyl, cyano, N—C₁₋₄alkylamino, N,N-di-C₁₋₄alkylamino, C₁₋₆alkyl-S(Oₙ)—, —O—R^{b}, —NR^{b}R^{c}, —C(O)—R^{b}, —C(O)O—R^{b}, —CONR^{b}R^{c}, NH—C(O)—R^{b} or —S(Oₙ)NR^{b}R^{c},
where R^{b} and R^{c} are independently selected from hydrogen and C₁₋₆alkyl optionally substituted with hydroxy, amino, N—C₁₋₄alkylamino, N,N-di-C₁₋₄alkylamino, HO—C₂₋₄alkyl-NH— or HO—C₂₋₄alkyl-N(C₁₋₄alkyl)-;
(ii) nitro when B is a group of Formula (IV) and X is CH and p is 0;
(iii) carbocyclyl (such as C₃₋₇cycloalkyl or aryl) or arylC₁₋₆alkyl each of which is optionally substituted by R¹², or R¹³;
(iv) heterocyclyl or heterocyclylC₁₋₆alkyl each of which is optionally substituted by up to 4 substituents independently selected from R¹² or R¹³, and where any nitrogen atoms within a heterocyclyl group are, where chemically allowed, optionally in their oxidised (N→O, N—OH) state;
A is selected from:
(i) a direct bond;
(ii) optionally substituted C₁₋₅alkylene wherein the optional substituents are independently selected from: hydroxy, hydroxyC₁₋₆alkyl, C₁₋₆alkyl, C₁₋₆alkoxy, C₁₋₄alkoxyC₁₋₄alkyl, aryl or arylC₁₋₆alkyl;
(iii) a carbocyclic ring of 3-7 atoms;
(iv) a carbonyl group or —C(O)—C(R^{d}R^{d})—, wherein R^{d} is independently selected from hydrogen and C₁₋₂alkyl;
or when R³ is a group of Formula (IIa) or (IIb), the group 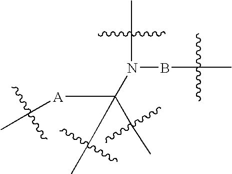
forms a heterocyclic ring containing 3-7 carbon atoms and one or more heteroatoms;
or when R³ is a group of Formula (IIa), (IIb), (IIc) or (IId), the group 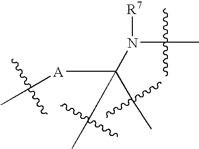
forms a heterocyclic ring containing 3-7 carbon atoms and one or more heteroatoms;
B is selected from:
(i) a direct bond;
(ii) a group of Formula (IV) 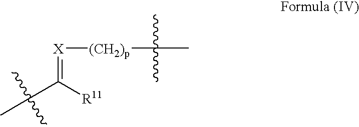
wherein:
X is selected from N or CH,
wherein at position (a) Formula (IV) is attached to the nitrogen atom and the (CH₂)ₚ group is attached to R⁸; and
(iii) a group independently selected from: optionally substituted C₁₋₆alkylene, optionally substituted C₃₋₇cycloalkyl, optionally substituted C₃₋₆alkenylene, optionally substituted C₃₋₆alkynyl, (C₁₋₅alkyl)ₐₐ-S(Oₙ)—(C₁₋₅alkyl)_{bb}-, —(C₁₋₅alkyl)ₐₐ-O—(C₁₋₅alkyl)_{bb}-, —(C₁₋₅alkyl)ₐₐ-C(O)—(C₁₋₅alkyl)_{bb}- or (C₁₋₅alkyl)ₐₐ-N(R¹⁷)—(C₁₋₅alkyl)_{bb}, or —(C₁₋₅alkyl)ₐₐ-C(O)NH—(C₁₋₅alkyl)_{bb}-
where R¹⁷ is hydrogen or C₁₋₄alkyl, or where R¹⁷ and the (C₁₋₅alkyl)ₐₐ or (C₁₋₅alkyl)_{bb} chain can be joined to form a heterocyclic ring, wherein aa and bb are independently 0 or 1 and the combined length of (C₁₋₅alkyl)ₐₐ and (C₁₋₅alkyl)_{bb} is less than or equal to C₁₋₅alkyl and wherein the optional substituents are independently selected from R¹²;
or the group —B—R⁸ represents a group of Formula (V) 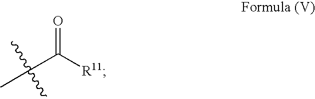
or the group 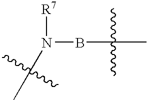
together forms an optionally substituted heterocyclic ring containing 4-7 carbons atoms, wherein the optional substituents are selected from 1 or 2 substituents independently selected from R¹² and R¹³;
or the group 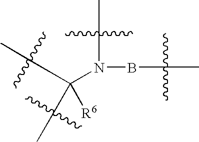
forms a heterocyclic ring containing 3-7 carbon atoms and one or more heteroatoms;
R¹¹ is selected from: hydrogen, optionally substituted C₁₋₆alkyl, N(R²³R²⁴) or NC(O)OR²⁵, where R²³, R²⁴ and R²⁵ are independently selected from: hydrogen, hydroxy, optionally substituted C₁₋₆alkyl, optionally substituted aryl, optionally substituted arylC₁₋₆alkyl, an optionally substituted carbocyclic ring of 3-7 atoms, optionally substituted heterocyclyl or optionally substituted heterocyclylC₁₋₆alkyl or R²³ and R²⁴ taken together with the nitrogen atom to which they are attached, can form an optionally substituted ring of 3-10 atoms, wherein the optional substituents are selected from R¹² and 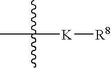
where K and R⁸ are as defined herein;
J is a group of the formula: —(CH₂)ₛ-L-(CH₂)ₛ— or —(CH₂)ₛ—C(O)—(CH₂)ₛ-L-(CH₂)ₛ— wherein when s is greater than 0, the alkylene group is optionally substituted,
or the group 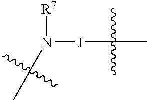
together forms an optionally substituted heterocyclic ring containing 4-7 carbons atoms, wherein the optional substituents are selected from 1 or 2 substituents independently selected from R¹² and R¹³;
K is selected from: a direct bond, —(CH₂)ₛ₁—, —(CH₂)ₛ₁—O—(CH₂)ₛ₂—, —(CH₂)ₛ₁—C(O)—(CH₂)ₛ₂—, —(CH₂)ₛ₁—S(Oₙ)—(CH₂)ₛ₂—, —(CH₂)ₛ₁—N(R^{17a})—(CH₂)ₛ₂—, —(CH₂)ₛ₁—C(O)N(R^{17a})—(CH₂)ₛ₂—, —(CH₂)ₛ₁—N(R^{17a})C(O)—(CH₂)ₛ₂—, —(CH₂)ₛ₁—N(R^{17a})C(O)N(R^{17a})—(CH₂)ₛ₂—, —(CH₂)ₛ₁—OC(O)—(CH₂)ₛ₂—, —(CH₂)ₛ₁—C(O)O—(CH₂)ₛ₂—, —(CH₂)ₛ₁—N(R^{17a})C(O)O—(CH₂)ₛ₂—, —(CH₂)ₛ₁—OC(O)N(R^{17a})—(CH₂)ₛ₂—, —(CH₂)ₛ₁—OS(Oₙ)—(CH₂)ₛ₂—, or —(CH₂)ₛ₁—S(Oₙ)—O—(CH₂)ₛ₂—, —(CH₂)ₛ₁—S(O)₂N(R^{17a})—(CH₂)ₛ₂— or —(CH₂)ₛ₁—N(R^{17a})S(O)₂—(CH₂)ₛ₂—; wherein the —(CH₂)ₛ₁— and —(CH₂)ₛ₂— groups are independently optionally substituted by hydroxy or C₁₋₄alkyl and wherein when s1>1 or s2>1 then the CH₂ group can optionally be a branched chain;
where R^{17a} is hydrogen or C₁₋₄alkyl;
L is selected from optionally substituted aryl or optionally substituted heterocyclyl;
R⁴ is selected from hydrogen, C₁₋₄alkyl or halo;
R⁵ is selected from a group of Formula III-a; III-b; III-c; III-d; III-e; III-f, III-g, III-h, III-i, or III-j, III-k, III-l, III-m, III-n or III-o 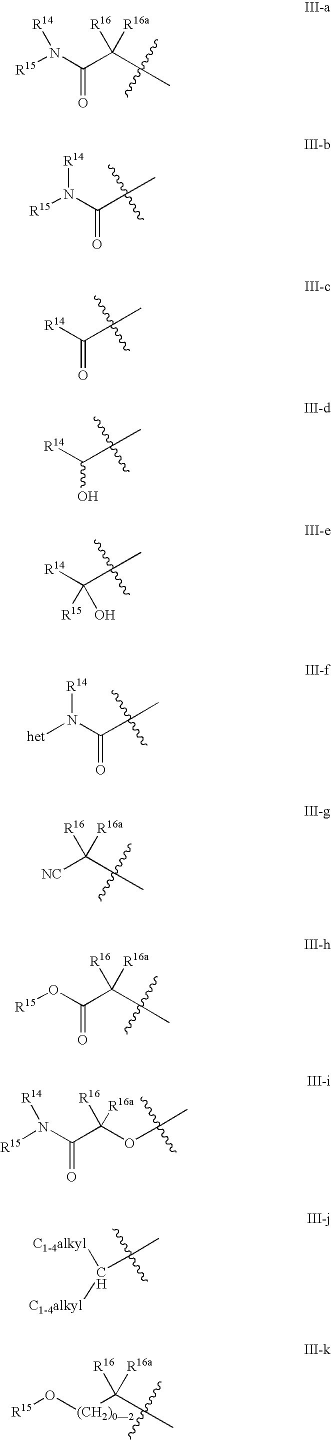 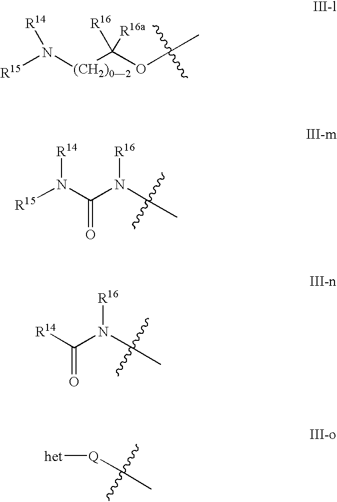
wherein:
het represents an optionally substituted 3- to 8-membered heterocyclic ring containing from 1 to 4 heteroatoms independently selected from O, N and S, wherein the optional substituents are selected from 1-2 groups selected from R¹² and R¹³; and
Q is selected from a direct bond or —[C(R¹⁶R^{16a})]₁₋₂—;
R¹⁴ and R¹⁵ are selected from:
(i) R¹⁴ selected from hydrogen; optionally substituted C₁₋₈alkyl; optionally substituted aryl; —R^{d}—Ar, where R^{d} represents C₁₋₈alkylene and Ar represents optionally substituted aryl; and optionally substituted 3- to 8-membered heterocyclic ring optionally containing from 1 to 3 further heteroatoms independently selected from O, N and S; and R¹⁵ is selected from hydrogen; optionally substituted C₁₋₈alkyl and optionally substituted aryl;
(ii) wherein the group of Formula (III) represents a group of Formula III-a, III-b, III-i, III-l or III-m, then the group NR¹⁴(—R¹⁵) represents an optionally substituted 3- to 8-membered heterocyclic ring optionally containing from 1 to 3 further heteroatoms independently selected from O, N and S; or
(iii) wherein the group of Formula (III) represents structure III-e, 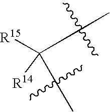
represents an optionally substituted 3- to 8-membered heterocyclic ring optionally containing from 1 to 4 heteroatoms independently selected from O, N and S;
R¹⁶ and R^{16a} are independently selected from:
(i) hydrogen or optionally substituted C₁₋₈alkyl; or
(ii) R¹⁶ and R^{16a} together with the carbon to which they are attached form an optionally substituted 3 to 7-membered cycloalkyl ring;
R¹² is independently selected from: halo, hydroxy, hydroxyC₁₋₆alkyl, oxo, cyano, cyanoC₁₋₆alkyl, nitro, carboxyl, C₁₋₆alkyl, C₁₋₆alkoxy, C₁₋₆alkoxyC₁₋₄alkyl, C₁₋₆alkoxycarbonylC₀₋₄alkyl, C₁₋₆alkanoylC₀₋₄alkyl, C₁₋₆alkanoyloxyC₀₋₄alkyl, C₂₋₆alkenyl, C₁₋₃perfluoroalkyl-, C₁₋₃perfluoroalkoxy, aryl, arylC₁₋₆alkyl, heterocyclyl, heterocyclylC₁₋₆alkyl, aminoC₀₋₄alkyl, N—C₁₋₄alkylaminoC₀₋₄alkyl, N,N-di-C₁₋₄alkylaminoC₀₋₄alkyl, carbamoyl, N—C₁₋₄alkylcarbamoylC₀₋₂alkyl, N,N-di-C₁₋₄alkylaminocarbamoylC₀₋₂alkyl, aminocarbonylC₀₋₄alkyl, N—C₁₋₆alkyaminocarbonylC₀₋₄alkyl, N,N—C₁₋₆alkyaminocarbonylC₀₋₄alkyl, C₁₋₆alkyl-S(O)ₙ-aminoC₀₋₄alkyl-, aryl-S(O)ₙ-aminoC₀₋₂alkyl-, C₁₋₃perfluoroalkyl-S(O)ₙ-aminoC₀₋₂alkyl-; C₁₋₆alkylamino-S(O)ₙ—C₀₋₂alkyl-, arylamino-S(O)ₙ—C₀₋₂alkyl-, C₁₋₃perfluoroalkylamino-S(O)ₙ—C₀₋₂alkyl-, C₁₋₆alkanoylamino-S(O)ₙ—C₀₋₂alkyl-; arylcarbonylamino-S(O)ₙ—C₀₋₂alkyl-, C₁₋₆alkyl-S(O)ₙ—C₀₋₂alkyl-, aryl-S(O)ₙ—C₀₋₂alkyl-, C₁₋₃perfluoroalkyl-, C₁₋₃perfluoroalkoxyC₀₋₂alkyl; R⁹′OC(O)(CH₂)_{w}—, R⁹″R¹⁰″N(CH₂)_{w}—, R⁹′R¹⁰′NC(O)(CH₂)_{w}—, R⁹R¹⁰NC(O)N(R⁹)(CH₂)_{w}—, R⁹OC(O)N(R⁹)(CH₂)_{w}—, or halo, wherein w is an integer between 0 and 4 and R⁹ and R¹⁰ are independently selected from hydrogen, C₁₋₄alkyl, C₁₋₄alkylsulphonyl and C₃₋₇carbocyclyl, R⁹′ and R¹⁰′ are independently selected from C₁₋₄alkylsulphonyl and C₃₋₇carbocyclyl, and R⁹″ and R¹⁰″ are C₃₋₇carbocyclyl; wherein an amino group within R¹² is optionally substituted by C₁₋₄alkyl;
R¹³ is C₁₋₄alkylaminocarbonyl wherein the alkyl group is optionally substituted by 1, 2 or 3 groups selected from R¹², or R¹³ is a group —C(O)—R¹⁸ and R¹⁸ is selected from an amino acid derivative or an amide of an amino acid derivative;
M is selected from —CH₂—CH₂— or —CH═CH—;
n is an integer from 0 to 2;
p is an integer from 0 to 4;
s, s1 and s2 are independently selected from an integer from 0 to 4, and
s1+s2 is less than or equal to 4;
t is an integer between 0 and 4; and
or a salt, solvate or pro-drug thereof to a patient.

2. A compound of formula (IA) which is a compound of formula (I): 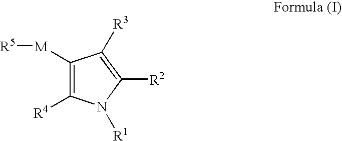
wherein:
R¹ is selected from: hydrogen, optionally substituted C₁₋₆alkyl, optionally substituted aryl or optionally substituted arylC₁₋₆alkyl, wherein the optional substituents are selected from C₁₋₄alkyl, nitro, cyano fluoro and C₁₋₄alkoxy;
R² is an optionally substituted mono or bi-cyclic aromatic ring wherein the optional substituents are 1, 2 or 3 substituents independently selected from: cyano, R^{e}R^{f}N—, C₁₋₆alkyl, C₁₋₆alkoxy, halo, haloC₁₋₆alkyl or haloC₁₋₆alkoxy wherein R^{e} and R^{f} are independently selected from hydrogen, C₁₋₆alkyl or aryl;
R³ is selected from a group of Formula (IIa) to Formula (IId): 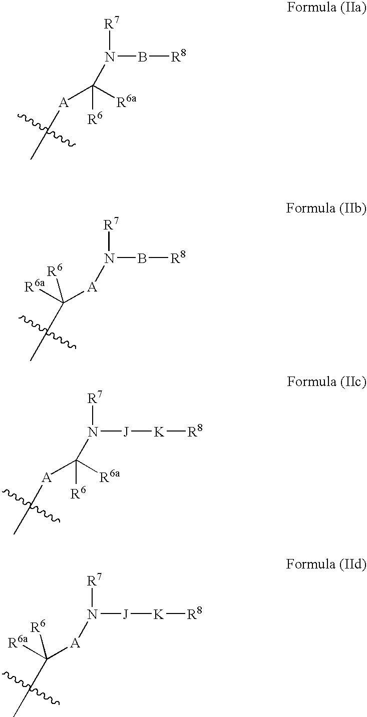
where R⁶ and R^{6a} are independently selected from hydrogen, fluoro, optionally substituted C₁₋₆alkyl, C₁₋₆alkoxy, or R⁶ and R^{6a} taken together and the carbon atom to which they are attached form a carbocyclic ring of 3-7 atoms or R⁶ and R^{6a} taken together and the carbon atom to which they are attached form a carbonyl group;
or when A is not a direct bond the group 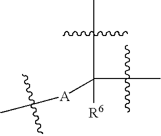
forms a carbocyclic ring of 3-7 carbon atoms or a heterocyclic ring containing one or more heteroatoms;
or the group 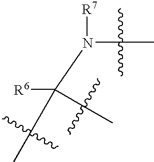
forms a heterocyclic ring containing 3-7 carbon atoms and one or more heteroatoms;
R⁷ is selected from: hydrogen or C₁₋₆alkyl;
R⁸ is selected from:
(i) hydrogen, C₁₋₆alkyl, C₂₋₆alkenyl, C₂₋₆alkynyl, haloC₁₋₆alkyl, C₁₋₄alkoxyC₁₋₄alkyl, hydroxy, hydroxyC₁₋₆alkyl cyano N—C₁₋₄alkylamino N,N-di-C₁₋₄alkylamino, C₁₋₆alkyl-S(Oₙ)—, O—R^{b}, —NR^{b}R^{c}, —C(O)—R^{b}, —C(O)O—R^{b}, —CONR^{b}R^{c}, NH—C(O)—R^{b} or —S(Oₙ)NR^{b}R^{c},
where R^{b} and R^{c} are independently selected from hydrogen and C₁₋₆alkyl optionally substituted with hydroxy, amino, N—C₁₋₄alkylamino N,N-di-C₁₋₄alkylamino, HO—C₂₋₄alkyl-NH— or HO—C₂₋₄alkyl-N(C₁₋₄alkyl)-;
(ii) nitro when B is a group of Formula (IV) and X is CH and p is 0;
(iii) carbocyclyl (such as C₃₋₇cycloalkyl or aryl) or arylC₁₋₆alkyl each of which is optionally substituted by R¹² or R¹³;
(iv) heterocyclyl or heterocyclylC₁₋₆alkyl each of which is optionally substituted by up to 4 substituents independently selected from R¹² or R¹³, and where any nitrogen atoms within a heterocyclyl group are, where chemically allowed, optionally in their oxidised (N→O, N—OH) state;
A is selected from:
(i) a direct bond;
(ii) optionally substituted C₁₋₅alkylene wherein the optional substituents are independently selected from: hydroxy, hydroxyC₁₋₆alkyl, C₁₋₆alkyl, C₁₋₆alkoxy, C₁₋₄alkoxyC₁₋₄alkyl, aryl or arylC₁₋₆alkyl;
(iii) a carbocyclic ring of 3-7 atoms;
(iv) a carbonyl group or —C(O)—C(R^{d}R^{d})—, wherein R^{d} is independently selected from hydrogen and C₁₋₂alkyl;
or when R³ is a group of Formula (IIa) or (IIb) the group 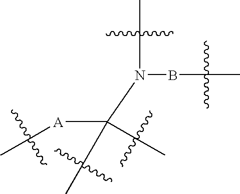
forms a heterocyclic ring containing 3-7 carbon atoms and one or more heteroatoms;
or when R³ is a group of Formula (IIa) (IIb) (IIc) or (IId) the group 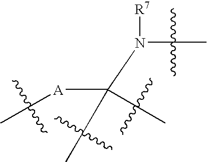
forms a heterocyclic ring containing 3-7 carbon atoms and one or more heteroatoms;
B is selected from:
(i) a direct bond;
(ii) a group of Formula (IV) 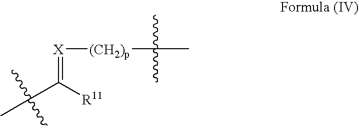
wherein:
X is selected from N or CH,
wherein at position (a) Formula (IV) is attached to the nitrogen atom and the (CH₂)ₚ group is attached to R⁸; and
(iii) a group independently selected from: optionally substituted C₁₋₆alkylene, optionally substituted C₁₋₇cycloalkyl optionally substituted C₃₋₆alkenylene, optionally substituted C₃₋₆alkynyl, (C₁₋₅alkyl)ₐₐ-S(Oₙ)—(C₁₋₅alkyl)_{bb}-, —(C₁₋₅alkyl)ₐₐ-O—(C₁₋₅alkyl)_{bb}-, —(C₁₋₅alkyl)ₐₐ-C(O)—(C₁₋₅alkyl)_{bb}- or (C₁₋₅alkyl)ₐₐ-N(R¹⁷)—(C₁₋₅alkyl)_{bb}, or —(C₁₋₅alkyl)ₐₐ-C(O)NH—(C₁₋₅alkyl)_{bb}-
where R¹⁷ is hydrogen or C₁₋₄alkyl, or where R¹⁷ and the (C₁₋₅alkyl)ₐₐ or (C₁₋₁₅alkyl)_{bb} chain can be joined to form a heterocyclic ring, wherein aa and bb are independently 0 or 1 and the combined length of (C₁₋₅alkyl)ₐₐ and (C₁₋₅alkyl)_{bb} is less than or equal to C₅alkyl and wherein the optional substituents are independently selected from R¹²;
or the group —B—R⁸ represents a group of Formula (V) 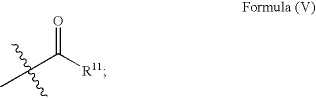
or the group 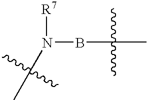
together forms an optionally substituted heterocyclic ring containing 4-7 carbons atoms, wherein the optional substituents are selected from 1 or 2 substituents independently selected from R¹² and R¹³;
or the group 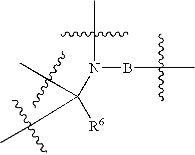
forms a heterocyclic ring containing 3-7 carbon atoms and one or more heteroatoms;
R¹¹ is selected from: hydrogen, optionally substituted C₁₋₆alkyl N(R²³R²⁴) or NC(O)OR²⁵ where R²³, R²⁴ and R²⁵ are independently selected from: hydrogen, hydroxy, optionally substituted C₁₋₆alkyl, optionally substituted aryl, optionally substituted arylC₁₋₆alkyl, an optionally substituted carbocyclic ring of 3-7 atoms, optionally substituted heterocyclyl or optionally substituted heterocyclylC₁₋₆alkyl or R²³ and R²⁴ taken together with the nitrogen atom to which they are attached, can form an optionally substituted ring of 3-10 atoms, wherein the optional substituents are selected from R¹² and 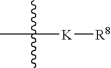
where K and R⁸ are as defined herein;
J is a group of the formula: —(CH₂)ₛ-L-(CH₂)ₛ— or —(CH₂)ₛ—C(O)—(CH₂)ₛ-L-(CH₂)ₛ— wherein when s is greater than 0 the alkylene group is optionally substituted,
or the group 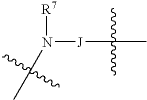
together forms an optionally substituted heterocyclic ring containing 4-7 carbons atoms, wherein the optional substituents are selected from 1 or 2 substituents independently selected from R¹² and R¹³;
K is selected from: a direct bond, —(CH₂)ₛ₁—, —(CH₂)ₛ₁—O—(CH₂)ₛ₁—C(O)—(CH₂)ₛ₂, —(CH₂)ₛ₁—S(Oₙ)—(CH₂)ₛ₂—, —(CH₂)ₛ₁—N(R^{17a})—(CH₂)ₛ₂, —(CH₂)ₛ₁—C(O)N(R^{17a})—(CH₂)ₛ₂, —(CH₂)ₛ₁—N(R^{17a})C(O)—(CH₂)ₛ₂—, —(CH₂)ₛ₁—N(R^{17a})C(O)N(R^{17a})—(CH₂)ₛ₂—, —(CH₂)ₛ₁—OC(O)—(CH₂)ₛ₂—, —(CH₂)ₛ₁—C(O)O—(CH₂)ₛ₂—, —(CH₂)ₛ₁—N(R^{17a})C(O)O—(CH₂)ₛ₂—, —(CH₂)ₛ₁—OC(O)N(R^{17a})—(CH₂)ₛ₂—, —(CH₂)ₛ₁—OS(Oₙ)—(CH₂)ₛ₂—, or —(CH₂)ₛ₁—S(Oₙ)—O—(CH₂)ₛ₂—, —(CH₂)ₛ₁—S(O)₂N(R^{17a})—(CH₂)ₛ₂— or —(CH₂)ₛ₁—N(R^{17a})S(O)₂—(CH₂)ₛ₂—; wherein the —(CH₂)ₛ₁— and —(CH₂)ₛ₂— groups are independently optionally substituted by hydroxy or C₁₋₄alkyl and wherein when s1>1 or s2>1 then the CH₂ group can optionally be a branched chain;
where R^{17a} is hydrogen or C₁₋₄alkyl;
L is selected from optionally substituted aryl or optionally substituted heterocyclyl;
R⁴ is selected from hydrogen, C₁₋₄alkyl or halo;
R⁵ is selected from a group of Formula III-a; III-b; III-c; III-d; III-e; III-f III-g, III-h, III-i or III-j, III-k, III-l, III-m, III-n or III-o 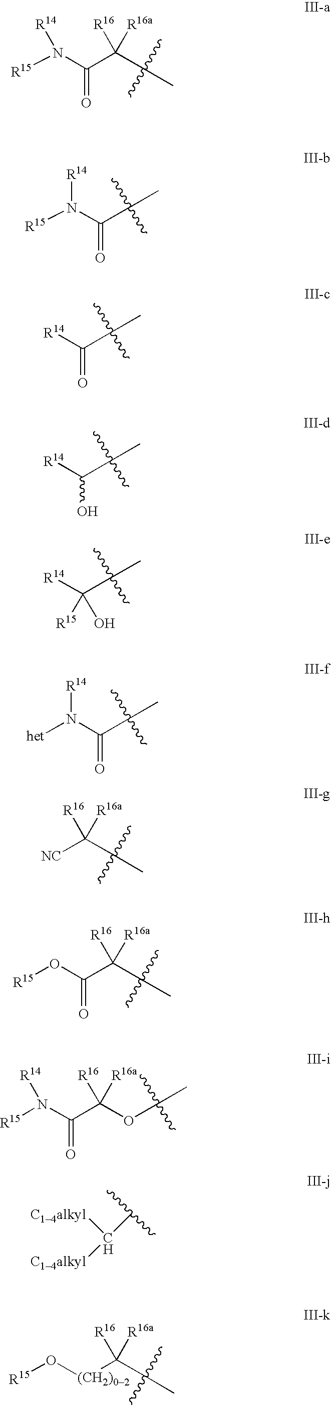 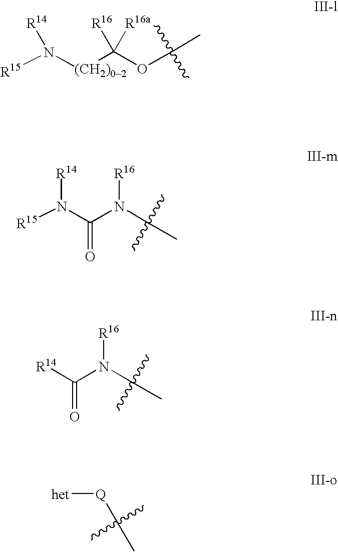
wherein:
het represents an optionally substituted 3- to 8-membered heterocyclic ring containing from 1 to 4 heteroatoms independently selected from O, N and S, wherein the optional substituents are selected from 1-2 groups selected from R¹² and R¹³; and
Q is selected from a direct bond or —[C(R¹⁶R^{16a})]₁₋₂—;
R¹⁴ and R¹⁵ are selected from:
(i) R¹⁴ selected from hydrogen; optionally substituted C₁₋₈alkyl; optionally substituted aryl; —R^{d}—Ar, where R^{d} represents C₁₋₈alkylene and Ar represents optionally substituted aryl; and optionally substituted 3- to 8-membered heterocyclic ring optionally containing from 1 to 3 further heteroatoms independently selected from O, N and S; and R¹⁵ is selected from hydrogen; optionally substituted C₁₋₈alkyl and optionally substituted aryl;
(ii) wherein the group of Formula (III) represents a group of Formula III-a, III-b, III-i, III-l or III-m, then the group NR¹⁴(—R¹⁵) represents an optionally substituted 3- to 8-membered heterocyclic ring optionally containing from 1 to 3 further heteroatoms independently selected from O, N and S; or
(iii) wherein the group of Formula (III) represents structure III-e, 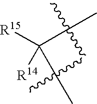
represents an optionally substituted 3- to 8-membered heterocyclic ring optionally containing from 1 to 4 heteroatoms independently selected from O, N and S;
R¹⁶ and R^{16a} are independently selected from:
(i) hydrogen or optionally substituted C₁₋₈alkyl; or
(ii) R¹⁶ and R^{16a} together with the carbon to which they are attached form an optionally substituted 3 to 7-membered cycloalkyl ring;
R¹² is independently selected from: halo, hydroxy, hydroxyC₁₋₆alkyl, oxo, cyano, cyanoC₁₋₆alkyl, nitro, carboxyl, C₁₋₆alkyl, C₁₋₆alkoxyC₁₋₆alkoxyC₁₋₄alkyl, C₁₋₆alkoxycarbonylC₀₋₄alkyl, C₁₋₆alkanoylC₀₋₄alkyl, C₁₋₆alkanoyloxyC₀₋₄alkyl, C₂₋₆alkenyl, C₁₋₃perfluoroalkyl-, C₁₋₃perfluoroalkoxy, aryl, arylC₁₋₆alkyl, heterocyclyl, heterocyclylC₁₋₆alkyl, aminoC₀₋₄alkyl, N—C₁₋₄alkylaminoC₀₋₄alkyl, N,N-di-C₁₋₄alkylaminoC₀₋₄alkyl carbamoyl, N—C₁₋₄alkylcarbamoylC₀₋₂alkyl, N, N-di-C₁₋₄alkylaminocarbamoylC₀₋₂alkyl, aminocarbonylC₀₋₄alkyl, N—C₁₋₆alkyaminocarbonylC₀₋₄alkyl, N,N—C₁₋₆alkyaminocarbonylC₀₋₄alkyl, C₁₋₆alkyl-S(O)ₙ-aminoC₀₋₄alkyl-, aryl-S(O)ₙ-aminoC₀₋₂alkyl-, C₁₋₃perfluoroalkyl-S(O)ₙ-aminoC₀₋₂alkyl-; C₁₋₆alkylamino-S(O)ₙ—C₀₋₂alkyl-, arylamino-S(O)ₙ—C⁰⁻²alkyl-, C₁₋₃perfluoroalkylamino-S(O)ₙ—C₀₋₂alkyl-, C₁₋₆alkanoylamino-S(O)ₙ—C₀₋₂alkyl-; arylcarbonylamino-S(O)ₙ—C₀₋₂alkyl, C₁₋₆alkyl-S(O)ₙ—C₀₋₂alkyl-, aryl-S(O)ₙ—C₀₋₂alkyl-, C₁₋₃perfluoroalkyl-, C₁₋₃perfluoroalkoxyC₀₋₂alkyl; R⁹′OC(O)(CH₂)_{w}, R⁹″R¹⁰″N(CH₂)_{w}—, R⁹′R¹⁰′NC(O)(CH₂)_{w}—, R⁹R¹⁰NC(O)N(R⁹)(CH₂)_{w}—, R⁹OC(O)N(R⁹)(CH₂)_{w}—, or halo, wherein w is an integer between 0 and 4 and R⁹ and R¹⁰ are independently selected from hydrogen, C₁₋₄alkyl, C₁₋₄alkylsulphonyl and C₃₋₇carbocyclyl, R⁹′ and R¹⁰′ are independently selected from C₁₋₄alkylsulphonyl and C₃₋₇carbocyclyl, and R⁹″ and R¹⁰″ are C₃₋₇carbocyclyl; wherein an amino group within R¹² is optionally substituted by C₁₋₄alkyl;
R¹³ is C₁₋₄alkylaminocarbonyl wherein the alkyl group is optionally substituted by 1, 2 or 3 groups selected from R¹² or R¹³ is a group —C(O)—R¹⁸ and R¹⁸ is selected from an amino acid derivative or an amide of an amino acid derivative;
M is selected from —CH₂—CH₂— or —CH═CH—;
n is an integer from 0 to 2;
p is an integer from 0 to 4;
s, s1 and s2 are independently selected from an integer from 0 to 4 and
s1+s2 is less than or equal to 4;
t is an integer between 0 and 4; and
or a salt solvate or pro-drug thereof;
with the proviso that when
(i) the group 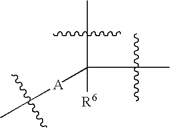
forms an aromatic carbocyclic ring of 3-7 carbon atoms or an aromatic heterocyclic ring containing one or more heteroatoms, or
(ii) when R³ is a group of Formula (IIa) or (IIb), and the group 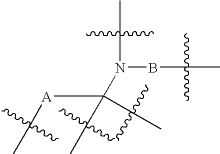
forms an aromatic heterocyclic ring containing 3-7 carbon atoms and one or more heteroatoms; or
(iii) when R³ is a group of Formula (IIa), (IIb), (IIc) or (IId), and the group 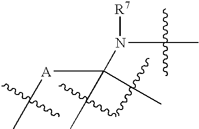
forms an aromatic heterocyclic ring containing 3-7 carbon atoms and one or more heteroatoms, or
(iv) when the group 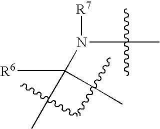
forms an aromatic heterocyclic ring containing 3-7 carbon atoms and one or more heteroatoms and A is a direct bond;
then R⁵ is other than a group III-o.

3. A compound according to claim 2 wherein the group A is selected from (i) a direct bond or (ii) optionally substituted C₁₋₅alkylene wherein the optional substituents are independently selected from: hydroxy, hydroxyC₁₋₆alkyl, C₁₋₆alkyl, C₁₋₆alkoxy, C₁₋₄alkoxyC₁₋₄alkyl, aryl or arylC₁₋₆alkyl.

4. A compound according to claim 2 which includes a group R¹³ and wherein the group R¹³ is —C(O)—R¹⁸, and R¹⁸ is selected from an amino acid derivative or an amide of an amino acid derivative; or a salt, solvate or pro-drug thereof.

5. A compound according to claim 2 wherein R¹ is selected from hydrogen, optionally C₁₋₆alkyl or optionally substituted arylC₁₋₆alkyl, wherein the optional substituents are selected from: fluoro and C₁₋₄alkoxy.

6. A compound according to claim 2 wherein R² is phenyl, optionally substituted by one or more groups selected from methyl, ethyl, methoxy, ethoxy, tert-butoxy, F or Cl.

7. A compound according to claim 2 wherein R³ is selected from a group of formula (IIc) or formula (IId).

8. A compound according to claim 2 wherein R⁴ is selected from hydrogen, methyl, ethyl, chloro or bromo.

9. A compound according to claim 2 wherein R⁵ is selected from a group of Formula III-a, III-g, III-h, III-i, III-j, III-k, III-l: or III-o 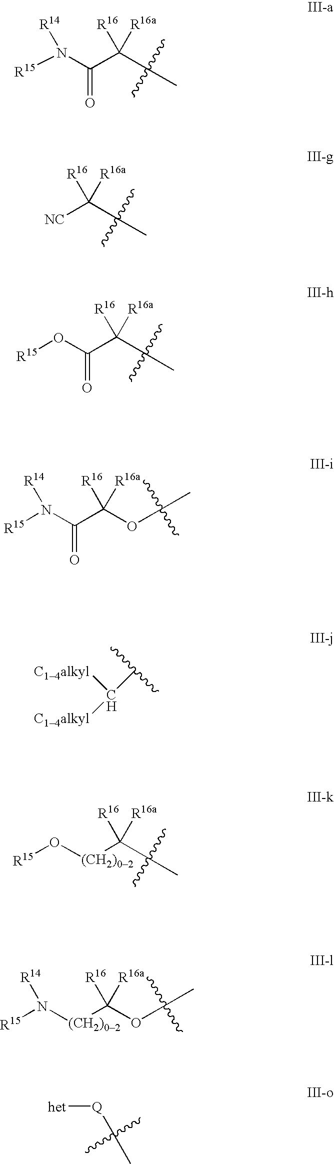
wherein R¹⁶, R^{16a}, R¹⁴ and R¹⁵ are as defined in claim 2.

10. A compound according to claim 9 wherein R⁵ is a group of formula

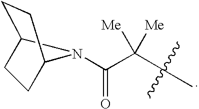

11. A compound according to claim 2 wherein M is —CH₂—CH₂—.

12. A compound of Formula (Ia) as claimed in claim 2 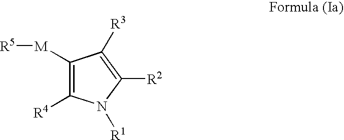
wherein:
R³ is selected from a group of Formula (IIa) or Formula (IIb): 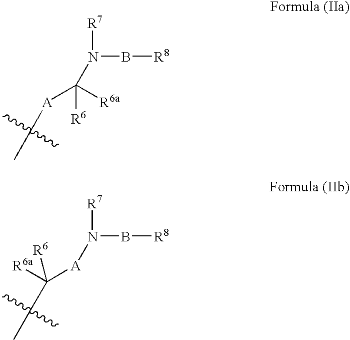
R⁷ is selected from: hydrogen or C₁₋₆alkyl;
B is a group of Formula (IV) 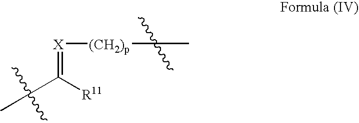
and p, A, X, M, R¹, R², R⁴, R⁵ R⁶, R^{6a}, R⁸, and R¹¹ are as defined in claim 2
or a salt, solvate or pro-drug thereof.

13. A compound of Formula (Ic) which is a compound of formula (Ia) as claimed in claim 2 wherein: 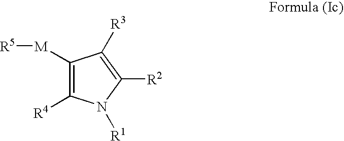
wherein:
R³ is selected from a group of Formula (IIc) or Formula (IId): 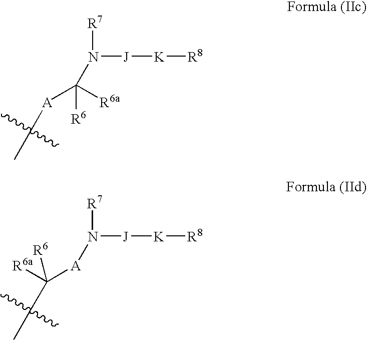
wherein
the group 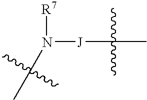
together forms an optionally substituted heterocyclic ring containing 4-7 carbons atoms, wherein the optional substituents are selected from 1 or 2 substituents independently selected from R¹² and R¹³;
and A, M, J, R¹, R², R⁴, R⁵ R⁶, R^{6a}, R⁸, and R¹² and R¹³ are as defined in claim 2,
or a salt, solvate or pro-drug thereof.

14. A compound selected from:
3-[3,3-dimethyl-4-oxo-4-(azabicyclo[2.2.1]heptan-7-yl)butyl]-4-[2-{4-(morpholin-4-ylcarbonyl)piperidin-1-yl}ethyl]-5-(3,5-dimethylphenyl)-1H-pyrrole;
3-[3,3-dimethyl-4-oxo-4-(azabicyclo[2.2.1]heptan-7-yl)but-2-en-1-yl]-4-[1s-methyl-2-(n′-isopropoxycarbonyl-3-pyrid-4-yl-pyrrolidin-1-ylcarboximidamido)ethyl]-5-(3,5-dimethylphenyl)-1h-pyrrole;
3-[3,3-dimethyl-4-oxo-4-(azabicyclo[2.2.1]heptan-7-yl)butyl]-4-[1S-methyl-2-(N′-isopropoxycarbonyl-3-pyrid-4-yl-pyrrolidin-1-ylcarboximidamido)ethyl]-5-(3,5-dimethylphenyl)-1H-pyrrole;
3-[3,3-dimethyl-4-oxo-4-(azabicyclo[2.2.1]heptan-7-yl)butyl]-4-[2-{4-(pyrrolidin-1-ylcarbonyl)piperazin-1-yl}ethyl]-5-(3,5-dimethylphenyl)-1H-pyrrole;
2-chloro-3-[3,3-dimethyl-4-oxo-4-(azabicyclo[2.2.1]heptan-7-yl)butyl]-4-[2-{4-(pyrrolidin-1-ylcarbonyl)piperazin-1-yl}ethyl]-5-(3,5-dimethylphenyl)-1h-pyrrole;
3-[3,3-dimethyl-4-oxo-4-(azabicyclo[2.2.1]heptan-7-yl)butyl]-4-[2-{4-(4-hydroxypiperidin-1-ylcarbonyl)piperidin-1-yl}ethyl]-5-(3,5-dimethylphenyl)-1h-pyrrole;
3-[3,3-dimethyl-4-oxo-4-(azabicyclo[2.2.1]heptan-7-yl)butyl]-4-[2-{4-(1,1-dioxo-isothiazolidin-2-ylcarbonyl)-4-methoxy-piperidin-1-yl}ethyl]-5-(3,5-dimethylphenyl)-1h -pyrrole;
3-[3,3-dimethyl-4-oxo-4-(azabicyclo[2.2.1]heptan-7-yl)butyl]-4-[1s-methyl-2-{1-benzyl-pyrrodin-3-ylamino}ethyl]-5-(3,5-dimethylphenyl)-1h-pyrrole;
3-[3,3-dimethyl-4-oxo-4-(azabicyclo[2.2.1]heptan-7-yl)butyl]-4-[1s-methyl-2-(2-{4-n-isopropylureidophenyl}ethylamino)ethyl]-5-(3,5-dimethylphenyl)-1h-pyrrole;
3-[3,3-dimethyl-4-oxo-4-(azabicyclo[2.2.1]heptan-7-yl)butyl]-4-[1s-methyl-2-{4-(pyrid-4-yl)piperidin-1-ylcarbonylamino}ethyl]-5-(3,5-dimethylphenyl)-1h-pyrrole;
3-[3,3-dimethyl-4-oxo-4-(azabicyclo[2.2.1]heptan-7-yl)butyl]-4-[1s-methyl-2-{3-(pyrid-4-yl)pyrrolidin-1-ylcarbonylamino}ethyl]-5-(3,5-dimethylphenyl)-1h-pyrrole; and
3-[3,3-dimethyl-4-oxo-4-(azabicyclo[2.2.1]heptan-7-yl)butyl]-4-[1s-methyl-2-{4-phenylpiperidin-1-ylcarbonylamino}ethyl]-5-(3,5-dimethylphenyl)-1h-pyrrole.

15. A process for preparing a compound of formula (I) as defined in claim 2 said process comprising a step selected from (a) to (h):
(a) reaction of a compound of formula XXXII with a compound of formula H—R³′, 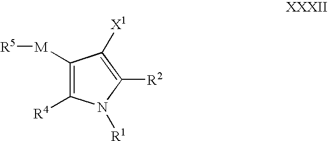
wherein X¹ is selected from: 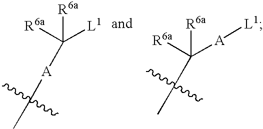
L¹ is a displaceable group; and
H—R³′ is selected from: 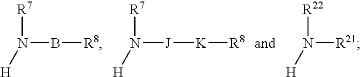
(b) reaction of a compound of formula XXXIII with a compound of formula L²-R³″, 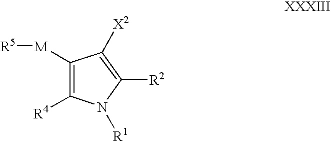
wherein X² is selected from: 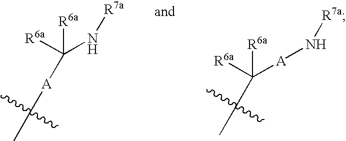
L² is a displaceable group and R^{7a} is selected from the definition of R⁷ or R²² above, and
L²-R³″ is selected from: L²-B—R⁸, L²-J-K—R⁸ and L²-R²¹
(c) for compounds of Formula (I) or (IA) wherein R⁷ is other than part of a heterocyclic ring or hydrogen, reaction of a compound of Formula (I) or (IA) wherein R³ is a group of Formula (IIa), (IIb), (IIc) or (IId) and R⁷ is hydrogen with a group of formula L³-R^{7a}, wherein R^{7a} is as defined above for R⁷ with the exclusion of hydrogen and L³ is a displaceable group;
(d) for compounds of Formula (I) or (IA) wherein R⁴ is hydrogen, reduction of a thienopyrrole of Formula XXXVIII 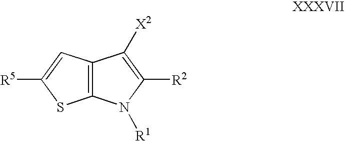
(e) for compounds of Formula (I) wherein R³ is a group of Formula (IIc) or (IId) and the group 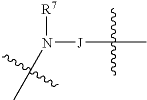
together forms an optionally substituted nitrogen-containing heterocyclic ring containing 4-7 carbons atoms, reaction of a compound of Formula XXXIVa or XXXIVb, with a compound of Formula L⁶-K—R⁸, wherein L⁶ is a displaceable group 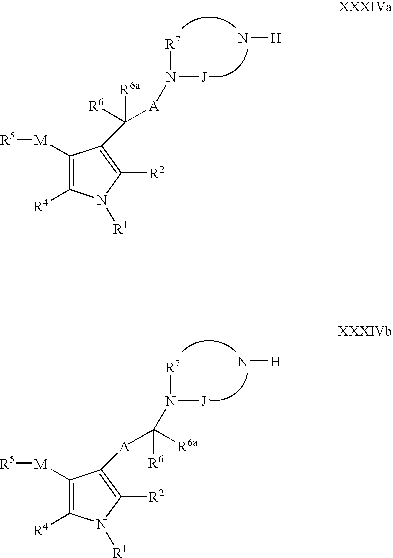
(f) for compounds of Formula (I) wherein R³ is a group of Formula (IIc) or (IId), reaction of a compound of Formula XXXVa or XXXVb, with a compound of Formula L⁷-K″—R⁸,
wherein L⁷ is a displaceable group, and wherein the groups K′ and K″ comprise groups which when reacted together form K, 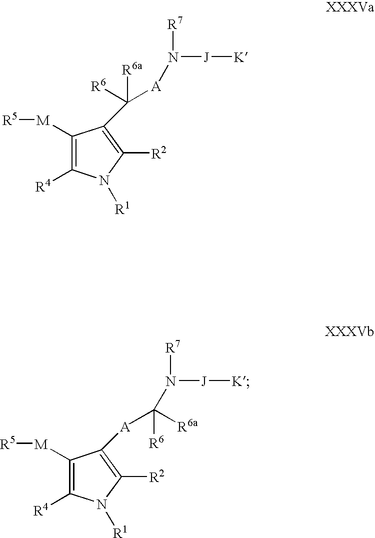
(g) reaction of a compound of Formula XXXVI with an electrophilic compound of the formula L⁸-R³, wherein L⁸ is a displaceable group 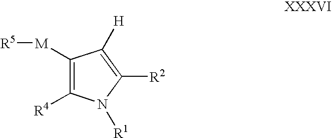
(h) reaction of a compound of Formula XXXIX with an appropriate electrophilic reagent to give a compounds of Formula (I) 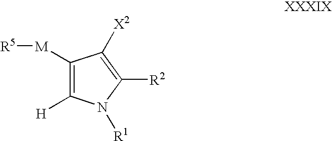
and thereafter if necessary, carrying out one or more of the following steps:
i) converting a compound of the Formula (I) into another compound of the Formula (I);
ii) removing any protecting groups;
iii) forming a salt, pro-drug or solvate.

16. A pharmaceutical formulation comprising a compound according to claim 2, or salt, pro-drug or solvate thereof, and a pharmaceutically acceptable diluent or carrier.

17-18. (canceled)
